SEQUENCE LISTING (21 sequences)

SEQ ID NO: 1 - DNA, 79 bp, Sus scrofa
  misc_feature (4)..(4): n is a, c, g, or t
tttngtatgctgaaacttctcaaccagaagaaagggccttcacagtggtcctttgtgtaagaatgatataaccaaaagg

SEQ ID NO: 2 - DNA, 2783 bp, Sus scrofa
ggtccaggatgtttctcttcaagcaaatgtaatgattctgatgtttcaatatttaaggtagaaaattatagcagtgataaaagtttaagtgagaaatacaataaatgccaactgatactaaaaaataacattgaaaggactgctgacatttttgttgaagaaaatactgacggttacaagagaaatactgaaaataaagacaacaaatgtactggtcttgctagtaacttaggaggaagctggatggacagtgcttcaagtaaaactgatacagtttatatgcacgaagatgaaactggtttgccatttattgatcacaacatacatctaaaattacctaaccactttatgaagaagggaaatactcaaattaaagaaggtttgtcagatttgacttgtttggaagttatgagagccgaagaaacatttcatattaatacatcaaataaacagtcaactgttaataagaggagccaaaaaataaaagattttgatgtttttgatttgtcctttcagagtgcaagtgggaaaaacatcagagtctctaaagagtcattaaataaagctgtaaatttctttgacgaaaaatgcacagaagaagaattgaataacttttcagattcctcaaattctgaaatacttcctggcataaatatcaacaaaataaacatttcaagccataaggaaacagattcggacaaaaacaaactattgaaagaaagtgacccagttggtattgaaaatcaattactgactctccagcaaagatcagaatgtgaaatcaaaaagatcgaagaacctaccatgctgggttttcatacagctagtgggaaaaaagtaaaaattgcgaaggaatcgttggacaaagtgaaaaatctttttgatgaaacaaagcaagatagtagtgaaaccactaattctagccatcaaggggtaaaaacacagaaggacagagaggtatgtaaagaagagcttgaattaacattcgagacagttgaaataactgcctcaaagcatgaagaaatacggaattttttagaggagaaaaaacttgtttctaaggagatcaccatgccacccaggctcttacgtcatcatttacacagacaaactgaaaatctcagcatgtcaaacagtatccccctaaaaggtaaagtacatgaaaatatggaagaagaaacatcttgtcacacagatcagtccacttgttcagccattgaaaattcagcattaacattttacacaggacatggcagaaaaatttctgtgaatcaggcttccgtatttgaagccaaaaagtggcttagagaaggagaattggacgatcaaccagaaaacgtagattctgccaaggtcatatgtttaaaggaatatgctagggattatgtaggaaatcctttgtgtgggagtagttcaaacagtatcataactgaaaatgacaaaaatctccctgaaaaacaaaattcaacttatttaagtaacagtgtgtctaacaactattcataccattctgatttttgtcattccaatgaggtgctcagcaaatcagaatctctctcagaaaataaaattggtaattctgatactgagccagcagtgaagaatgtcaaagacagaaaagacacttgtttttctgaagagatatccaccgtaagagaagcaaacacacacccacaagctgtagatgaagacagctgggttcggaagcttgtgattaactctacaccatgcaaaaataaaaatacacctggtgaagtgtccaatctaattcaaataattttgagatagagccacctgcattcagtacaagtgggaacatagcctttgtttcacatgaaacagacgtgagagagaggtttgcagacaacaacaggaaggcgattaagcaaaacactgagagtatgtcaggctcttgccaaatgaaaattatgactggcgctcataaggcattgggtgattcagaggatgttattttccctaactctccagatagtgaagaacatattacacgttcacaggaggtttttcctgaaattcaaagtgaacaaattttacaacatgacccaagtgtatccggattggagaaagtttctgaaatgccaccttgtcatattaacttaaaaacttttgatatacataagtttgatatgaaaagacatcccatgtcagtctcttctatgaatgattgtggggtttttagcacagcaagtggaaaatctgtacaagtatcagatactgcattacaaaaagcgagacaagtattttctaagacagaagatgtggctaagccattcttttccagagcagttaaaagtgatgaagaacattcagacaagtacacaagagaagaaaatgctatgatgcatccccccccaaatttcctgtcatctgctttctccggatttagtacagcaagtggaaaacaggttccagtttctgagagtgccttatgcaaagtgaagggaatgtttgaggaatttgatttaatgggaactgaatgtagacttcagcattcacctacatctagacaagatgtgtcaaagatacttcctctctccgagattgatgagagaaccccagaacactctgtaagttcccaaacagagaaagcctacaatgaacaatttaaattaccagatagctgtaacactgaaagcagttcttcagaaaataatcactctgttaaagtttctcccgatctctctcggtttaagcaagacaaacagttggtatcaggagcaaaagtatcacttgttgagaacattcatccatcgggaaaagaa

SEQ ID NO: 3 - DNA, 679 bp, Sus scrofa
agcatgagaccagcagtttattactcactaaagacagaatgaatgtagaaaaggctgaattttgtaataaaagcaagcagcctgtcttagcaaagagccaacagagcagatgggctgaaagtaagggcacatgtaatgataggcagactcctaacacagagaaaaaggtagttctgaatactgatctcctgtatgggagaaacgaactgaataagcagaaacctgcgtgctctgacagtcctagagattcccaagatgttccttggataacattgaatagtagcatacagaaagttaatgagtggttttctagaagcgatgaaatgttaacttctgacgactcacaggacaggaggtctgaatcaaatactggggtagctggtgcagcagaggttccaaatgaagcagatggacatttgggttcttcagagaaaatagacttaatggccagtgaccctcatggtgctttaatacgtgaacgtgaaagagggcactccaaaccagcagagagtaatattgaagataaaatatttgggaaaacctatcggaggaaggcaagcctccctaacttgagccacgtaattgaagatctaattttaggagcatctgctgtagagcctcaaataacacaagagcgccccctcacaaataaactaaagcggaaaaggagaggtacatc

SEQ ID NO: 4 - DNA, 1359 bp, Rattus rattus
atggttcgaaattttcggtatgggaagccagtccagagtcaagtacagctgaaaggccgtgacctcctcaccctgaagaacttcacaggagaggagattcagtacatgctatggctctctgcagatctgaaattcaggatcaaacagaaaggagaatacttgcctttattgcaagggaaatccttagggatgatttttgagaaaagaagtactcgaacaagactgtccacagaaacaggcttcgctcttctgggaggacatccttcttttcttaccacacaagacattcacttgggcgtgaatgaaagtctcacagacacagctcgtgtgttatctagcatgacagatgcagtgttagctcgagtgtataaacaatcagatctggacatcctggctaaggaagcaaccatcccaattgtcaacggactgtcagacctgtatcatcctatccagatcctggctgattaccttacactccaggaacactatggctctctcaaaggtctcaccctcagctggataggagatgggaacaatatcctgcactccatcatgatgagtgctgcaaaattcgggatgcaccttcaagcagctactccaaagggttatgagccagatcctaatatagtcaagctagcagagcagtatgccaaggagaatggtaccaggttgtcaatgacaaatgatccactggaagcagcacgtggaggcaatgtattaattacagatacttggataagcatgggacaagaggatgagaagaaaaagcgtcttcaagctttccaaggttaccaggttacaatgaagactgctaaagtggctgcgtctgactggacgtttttacactgcttgcctagaaagccagaagaagtagatgatgaagtgttttattctccgcggtcattagtgttcccagaggcagaaaatagaaagtggacaatcatggctgtcatggtatccctgctgacagactactcacctgtgctccagaagccaaagttctgatgcctgcaagaggacgaaaaacccaaaagacaaaaaaatctgttctttagcagcagaataagtcagtttatgtagaaaagagaagaattgaaattgtaaacacatccctagtgcgtgatataattatgtaattgctttgctattgtgagaattgcttaaagcttttagtttaagtgctgggcattttattatcctgcttgacttgacttaagcactctcttcaattcacaacttctgaatgatatttgggtttcatattaattatcatacacatttccttccactaagcattaaacactatgcttacaatgcataccatctaagtcattaaatgtaatccatgcttattacctt

SEQ ID NO: 5 - protein, 334 aa, Rattus rattus
MVRNFRYGKPVQSQVQLKGRDLLTLKNFTGEEIQYMLWLSADLKFRIKQKGEYLPLLQGKSLGMIFEKRSTRTRLSTETGFALLGGHPSFLTTQDIHLGVNESLTDTARVLSSMTDAVLARVYKQSDLDILAKEATIPIVNGLSDLYHPIQILADYLTLQEHYGSLKGLTLSWIGDGNNILHSIMMSAAKFGMHLQAATPKGYEPDPNIVKLAEQYAKENGTRLSMTNDPLEAARGGNVLITDTWISMGQEDEKKKRLQAFQGYQVTMKTAKVAASDWTFLHCLPRKPEEVDDEVFYSPRSLVFPEAENRKWTIMAVMVSLLTDYSPVLQKPKF

SEQ ID NO: 6 - DNA, 1927 bp, Homo sapiens
gagccccaggactgagatatttttactataccttctctatcatcttgcacccccaaaatagcttccagggcacttctatttgtttttgtggaaagactggcaattagaggtagaaaagtgaaataaatggaaatagtactactcagggctgtcacatctacatctgtgtttttgcagtgccaatttgcattttctgagtgagttacttctactcaccttcacagcagccagtaccgcagtgccttgcatatattatatcctcaatgagtacttgtcaattgattttgtacatgcgtgtgacagtataaatatattatgaaaaatgaggaggccaggcaataaaagagtcaggatttcttccaaaaaaaatacacagcggtggagcttggcataaagttcaaatgctcctacaccctgccctgcagtatctctaaccaggggactttgataaggaagctgaagggtgatattacctttgctccctcactgcaactgaacacatttcttagtttttaggtggcccccgctggctaacttgctgtggagttttcaagggcatagaatcgtcctttacacaattaaaagaagatgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggctttgcacttctgggaggacatccttgttttcttaccacacaagatattcatttgggtgtgaatgaaagtctcacggacacggcccgtgtattgtctagcatggcagatgcagtattggctcgagtgtataaacaatcagatttggacacccttgctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattttgatgttgtgttacttgtcaagaaagaagcaatgttcttcagtaacagaatgagttggtttatggggaaaagagaagagaatctaaaaaataaacaaatccctaacacgtggtatgggtgaaccgtatgatatgctttgccattgtgaaactttccttaagcctttaatttaagtgctgatgcactgtaatacgtgcttaactttgcttaaactctctaattcccaatttctgagttacatttagatatcatattaattatcatatacatttacttc

SEQ ID NO: 7 - protein, 354 aa, Homo sapiens
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFPTTQDIHLGVNESLTDTARVLSSMADAVLARVYKQSDLDTLAKEASIPIINGLSDLYHPIQILADYLTLQEHYSSLKGLTLSCFGDGNNILHSIMMSAAKFGMHLQAATPKGYEPDASVTKLAEQYAKENGTKLLLTNDPLEAAHGGNVLITDTWISMGREEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 8 - DNA, 1085 bp, Sus scrofa
ggtggacaaccactacaaaataaagtgcagctgaagggtcgtgacctcctcactctaaagaactttacaggagaagaaattaagtatatcctatggctatcagctgatctgaaatttaggataaagcagaaaggagagtatttgcctttattgcaagggaagtccctcggcatgatttttgaaaaaagaagtactcgaacaagattgtctacagaaacaggctttgcccttctaggaggacacccttgttttcttaccacacaagatattcacttgggtgtgaatgaaagtctcaaggacaccgctcgtgtgttgtctagcatgacagatgcagtgttggcgcgagtgtataaacaatcagacctggacatcctggctcaagaagcatccatcccaatcatcaatgggctgtcagatttgtaccatcctatccagatcctggctgattacctcacgctccaggaacactacggcgctctgaaaggccttaccctcagctggattggggatgggaataacatcctgcactccatcatgatgagtgcggcgaaatttgggatgcaccttcaggtggcgactccaaagggctatgagccggatcccagtataaccaagttggcggagcagtatgccaaggagaacggtaccaacgtgtcgctgacgaatgatccattggaggcggctcgtggaggcaatgtattaattacagacacttggataagcatgggacaagaagaggagaagaaaaagcggctccaggctttccaaggttaccaggttacaatgaagactgcggaagttgctgcctctgactggacatttttacactgcctgcccagaaagccagaagaagtggacgatgaagtgttttactctccacaatcacttgtattcccggaggctgaaaacagaaagtggacaatcatggctgtcatggtgtctctgctgacagattactcgcctcagctccagaagccgaagttttgatgccgtgatgcttgtcaagagggaaaccatgttctcccataacagaatgagtcagttgataagggaaggagaagagaatctaagaaataaacagatcc

SEQ ID NO: 9 - protein, 328 aa, Sus scrofa
GGQPLQNKVQLKGRDLLTLKNFTGEEIKYILWLSADLKFRIKQKGEYLPLLQGKSLGMIFEKRSTRTRLSTETGFALLGGHPCFLTTQDIHLGVNESLKDTARVLSSMTDAVLARVYKQSDLDILAQEASIPIINGLSDLYHPIQILADYLTLQEHYGALKGLTLSWIGDGNNILHSIMMSAAKFGMHLQVATPKGYEPDPSITKLAEQYAKENGTNVSLTNDPLEAARGGNVLITDTWISMGQEEEKKKRLQAFQGYQVTMKTAEVAASDWTFLHCLPRKPEEVDDEVFYSPQSLVFPEAENRKWTIMAVMVSLLTDYSPQLQKPKF

SEQ ID NO: 10 - DNA, 6307 bp, Artificial Sequence
  Plasmid vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccctacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttctgatagactaattgacatcatttgagtcaattggaggtgtacctgtggatgtatttcaagggaattctgtggaatgtgtgtcagttagggtgtggaaagtccccaggctccccaggcaggcagaagtatgcaaagcatcgaggatgtacgggccagatatacgcgataacttcgtataatgtatgctatacgaagttatcgcgtgaggttttcaccgtcatcaccgaaacgcgcgaggcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggctaccatggagaagttactattccgaagttcctattctctagaaagtataggaacttcaagcttggcactggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcggccaattgggccaccggtgctagccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgcccacaagacccgcagcgcccgaccgaaaggagcgcacgaccccatgcatcgaatcgatatcgcggccgcgactctagatcataatcagcccgggggtgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgacattctagttgtggtttgtccaaactcatcaatgtatcttatcatgtctggatcccatcacaaagctctgacctcaatcctatagaaaggaggaatgagccaaaattcacccaacttattgtgggaagcttgtggaaggctactcgaaatgtttgacccaagttaaacaatttaaaggcaatgctaccaaatactaattgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtagggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 11 - DNA, 6221 bp, Artificial Sequence
  Plasmid vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccctacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttctgatagactaattgacatcatttgagtcaattggaggtgtacctgtggatgtatttcaagggaattctgtggaatgtgtgtcagttagggtgtggaaagtccccaggctccccaggcaggcagaagtatgcaaagcatcgaggatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaaagcttgatatctaccatggagaagttactattccgaagttcctattctctagaaagtataggaacttcaagcttggcactggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcatagcggccgtaaattccgcccctctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaagatcccccgggggatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgacattctagttgtggtttgtccaaactcatcaatgtatcttatcatgtctggatcccatcacaaagctctgacctcaatcctatagaaaggaggaatgagccaaaattcacccaacttattgtgggaagcttgtggaaggctactcgaaatgtttgacccaagttaaacaatttaaaggcaatgctaccaaatactaattgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtagggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 12 - DNA, 6269 bp, Artificial Sequence
  Plasmid vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccctacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttctgatagactaattgacatcatttgagtcaattggaggtgtacctgtggatgtatttcaagggaattctgtggaatgtgtgtcagttagggtgtggaaagtccccaggctccccaggcaggcagaagtatgcaaagcatcgaggatgtacgggccagatatacgcgtgaggttttcaccgtcatcaccgaaacgcgcgaggcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggctaccatggagaagttactattccgaagttcctattctctagaaagtataggaacttcaagcttggcactggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcggccaattgggccaccggtgctagccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgcccacaagacccgcagcgcccgaccgaaaggagcgcacgaccccatgcatcgaatcgatatcgcggccgcgactctagatcataatcagcccgggggtgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgacattctagttgtggtttgtccaaactcatcaatgtatcttatcatgtctggatcccatcacaaagctctgacctcaatcctatagaaaggaggaatgagccaaaattcacccaacttattgtgggaagcttgtggaaggctactcgaaatgtttgacccaagttaaacaatttaaaggcaatgctaccaaatactaattgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtagggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 13 - DNA, 6346 bp, Artificial Sequence
  plasmid vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccctacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttctgatagactaattgacatcatttgagtcaattggaggtgtacctgtggatgtatttcaagggaattctgtggaatgtgtgtcagttagggtgtggaaagtccccaggctccccaggcaggcagaagtatgcaaagcatcgaggatgtacgggccagatatacgcgataacttcgtataatgtatgctatacgaagttatcgcgtgaggttttcaccgtcatcaccgaaacgcgcgaggcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggctaccatggagaagttactattccgaagttcctattctctagaaagtataggaacttcaagcttggcactggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcggccaattgggccaccggtgctagccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgcccacaagacccgcagcgcccgaccgaaaggagcgcacgaccccatgcatcgaatcgatatcgcggccgcgactctagatcataatcagcccgggggtgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggcgcgattaacttcgtataaagtctcctatacgaagttatcgcgccattctagttgtggtttgtccaaactcatcaatgtatcttatcatgtctggatcccatcacaaagctctgacctcaatcctatagaaaggaggaatgagccaaaattcacccaacttattgtgggaagcttgtggaaggctactcgaaatgtttgacccaagttaaacaatttaaaggcaatgctaccaaatactaattgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtagggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - DNA, 3791 bp, Homo sapiens
aagcttatattccatgctagggttctggtgttggtgcgtggggttggggtgggactgcagaagtgccttttaagattatgtgattgactgatctgtcattggttccctgccatctttatcttttggattcccctcggaggaggggaggaaggagtttcttttgggttttattgaatcaaatgaaagggaaagtagaggtgttcctatggaggggaggaaggagtttcttttgggttttattgaatcaaatgaaagggaaagtagaggtgttcctatgtcccgggctccggagcttctattcctgggccctgcataagaaggagacatggtggtggtggtggtgggtgggggtggtggggcacagaggaagccgatgctgggctctgcaccccattcccgctcccagatccctctggatatagcaccccctccagtgagcacagcctccccttgccccacagccaacagcaacatgcctcccaacaaagcatctgtccctcagccaaaacccctgttgcctctctctggggaaattgtagggctgggccagggtggggggaccattctctgcagggagattaggagtgtctgtcaggggcgggtggagcggggtggggccctggcttactcacatccttgagagtcctttgctggcagatttggggagcccacagctcagatgtctgtctcagcattgtcttccaagctcctaggccacagtagtggggcgctcccttctctggcttcttctttggtgacagtcaaggtggggttgggggtgacgaagggtcctgcttctcttctaggagcagttgatcccaggaagagcattggagcctccagcaggggctgttggggcctgtctgaggagataggatgcgtcaggcagccccagacacgatcacattcctctcaacatgcctgccggggtctgtggagccgaggggctgatgggagggtggggtgggggccggaagggtttgctttgggaggttgtctgggagattgctgaagttttgatatacacacctccaaagcaggaccaagtggactcctagaaatgtcccctgacccttggggcttcaggagtcagggaccctcgtgtccacctcagccttgcccttgcacagcccagctccactccagcctctactcctccccagaacatctcctgggccagttccacaaggggctcaaacgagggcacctgagctgcccacactagggatgttctgggggtctgagaagatatctggggctggaagaataaaaggcccccctaggcctgttcctggatgcagctccagccactttggggctaagcctgggcaataacaatgccaacgaggcttcttgccatactcggtttacaaaaccctttacatacattgtcgcattggattctcagagctgactgcactaagcagaatagatggtatgactcccactttgcagatgagaacactgaggctcagagaagtgcgaagccctgggtcacagaggcgtaaatgcagagccaggacccacctgaagacccacctgactccaggatgtttcctgcctccatgaggccacctgccctatggtgtggtggatgtgagatcctcaccatagggaggagattagggtctgtgctcagggctggggagaggtgcctggatttctctttgatggggatgttggggtgggaatcacgatacacctgatcagctgggtgtatttcagggatggggcagacttctcagcacagcacggcaggtcaggcctgggagggccccccagacctccttgtctctaatagagggtcatggtgagggaggcctgtctgtgcccaaggtgaccttgccatgccggtgctttccagccgggtatccatcccctgcagcagcaggcttcctctacgtggatgttaaaggcccattcagttcatggagagctagcaggaaactaggtttaaggtgcagaggccctgctctctgtcaccctggctaagcccagtgcgtgggttcctgagggctgggactcccagggtccgatgggaaagtgtagcctgcaggcccacacctccccctgtgaatcacgcctggcgggacaagaaagcccaaaacactccaaacaatgagtttccagtaaaatatgacagacatgatgaggcggatgagaggagggacctgcctgggagttggcgctagcctgtgggtgatgaaagccaaggggaatggaaagtgccagacccgccccctacccatgagtataaagcactcgcatccctttgcaatttacccgagcaccttctcttcactcagccttctgctcgctcgctcacctccctcctctgcaccatgactacctgcagccgccagttcacctcctccagctccatgaagggctcctgcggcatcgggggcggcatcgggggcggctccagccgcatctcctccgtcctggccggagggtcctgccgcgcccccagcacctacgggggcggcctgtctgtctcatcctcccgcttctcctctgggggagcctacgggctggggggcggctatggcggtggcttcagcagcagcagcagcagctttggtagtggctttgggggaggatatggtggtggccttggtgctggcttgggtggtggctttggtggtggctttgctggtggtgatgggcttctggtgggcagtgagaaggtgaccatgcagaacctcagtgaccgcctggcctcctacctggacaaggtgcgtgctctggaggaggccaacgccgacctggaagtgaagatccgtgactggtaccagaggcagcggcctgctgagatcaaagactacagtccctacttcaagaccattgaggacctgaggaacaagattctcacagccacagtggacaatgccaatgtccttctgcagattgacaatgcccgtctggccgcggatgacttccgcaccaagtatgagacagagttgaacctgcgcatgagtgtggaagccgacatcaatggcctgcgcagggtgctggacgaactgaccctggccagagctgacctggagatgcagattgagagcctgaaggaggagctggcctacctgaagaagaaccacgaggaggagatgaatgccctgagaggccaggtgggtggagatgtcaatgtggagatggacgctgcacctggcgtggacctgagccgcattctgaacgagatgcgtgaccagtatgagaagatggcagagaagaaccgcaaggatgccgaggaatggttcttcaccaagacagaggagctgaaccgcgaggtggccaccaacagcgagctggtgcagagcggcaagagcgagatctcggagctccggcgcaccatgcagaacctggagattgagctgcagtcccagctcagcatgaaagcatccctggagaacagcctggaggagaccaaaggtcgctactgcatgcagctggcccagatccaggagatgattggcagcgtggaggagcagctggcccagctccgctgcgagatggagcagcagaaccaggagtacaagatcctgctggacgtgaagacgcggctggagcaggagatcgccacctaccgccgcctgctggagggcgaggacgcccacctctcctcctcccagttctcctctggatcgcagtcatccagagatgtgacctcctccagccgccaaatccgcaccaaggtcatggatgtgcacgatggcaaggtggtgtccacccacgagcaggtccttcgcaccaagaacgactacaaggacgacgatgacaagtgaggatcc

SEQ ID NO: 15 - DNA, 1464 bp, Homo sapiens
atgactacctgcagccgccagttcacctcctccagctccatgaagggctctgcggcatcgggggcggcatcgggggcggctccagccgcatctcctccgtcctggccggagggtcctgccgcgcccccagcacctacgggggcggcctgtctgtctcatcctcccgcttctcctctgggggagcctacgggctggggggcggctatggcggtggcttcagcagcagcagcagcagctttggtagtggctttgggggaggatatggtggtggccttggtgctggcttgggtggtggctttggtggtggctttgctggtggtgatgggcttctggtgggcagtgagaaggtgaccatgcagaacctcagtgaccgcctggcctcctacctggacaaggtgcgtgctctggaggaggccaacgccgacctggaagtgaagatccgtgactggtaccagaggcagcggcctgctgagatcaaagactacagtccctacttcaagaccattgaggacctgaggaacaagattctcacagccacagtggacaatgccaatgtccttctgcagattgacaatgcccgtctggccgcggatgacttccgcaccaagtatgagacagagttgaacctgcgcatgagtgtggaagccgacatcaatggcctgcgcagggtgctggacgaactgaccctggccagagctgacctggagatgcagattgagagcctgaaggaggagctggcctacctgaagaagaaccacgaggaggagatgaatgccctgagaggccaggtgggtggagatgtcaatgtggagatggacgctgcacctggcgtggacctgagccgcattctgaacgagatgcgtgaccagtatgagaagatggcagagaagaaccgcaaggatgccgaggaatggttcttcaccaagacagaggagctgaaccgcgaggtggccaccaacagcgagctggtgcagagcggcaagagcgagatctcggagctccggcgcaccatgcagaacctggagattgagctgcagtcccagctcagcatgaaagcatccctggagaacagcctggaggagaccaaaggtcgctactgcatgcagctggcccagatccaggagatgattggcagcgtggaggagcagctggcccagctccgctgcgagatggagcagcagaaccaggagtacaagatcctgctggacgtgaagacgcggctggagcaggagatcgccacctaccgccgcctgctggagggcgaggacgcccacctctcctcctcccagttctcctctggatcgcagtcatccagagatgtgacctcctccagccgccaaatccgcaccaaggtcatggatgtgcacgatggcaaggtggtgtccacccacgagcaggtccttcgcaccaagaactgaggctgcccagccccgctcaggcctaggaggccccccgtgtggacac

SEQ ID NO: 16 - protein, 472 aa, Homo sapiens
MTTCSRQFTSSSSMKGSCGIGGGIGGGSSRISSVLAGGSCRAPSTYGGGLSVSSSRFSSGGAYGLGGGYGGGFSSSSSSFGSGFGGGYGGGLGAGLGGGFGGGFAGGDGLLVGSEKVTMQNLNDRLASYLDKVRALEEANADLEVKIRDWYQRQRPAEIKDYSPYFKTIEDLRNKILTATVDNANVLLQIDNARLAADDFRTKYETELNLRMSVEADINGLRRVLDELTLARADLEMQIESLKEELAYLKKNHEEEMNALRGQVGGDVNVEMDAAPGVDLSRILNEMRDQYEKMAEKNRKDAEEWFFTKTEELNREVATNSELVQSGKSEISELRRTMQNLEIELQSQLSMKASLENSLEETKGRYCMQLAQIQEMIGSVEEQLAQLRCEMEQQNQEYKILLDVKTRLEQEIATYRRLLEGEDAHLSSSQFSSGSQSSRDVTSSSRQIRTKVMDVHDGKVVSTHEQVLRTKN

SEQ ID NO: 17 - protein, 472 aa, Homo sapiens
MTTCSRQFTSSSSMKGSCGIGGGIGGGSSRISSVLAGGSCRAPSTYGGGLSVSSSRFSSGGAYGLGGGYGGGFSSSSSSFGSGFGGGYGGGLGAGLGGGFGGGFAGGDGLLVGSEKVTMQNLSDRLASYLDKVRALEEANADLEVKIRDWYQRQRPAEIKDYSPYFKTIEDLRNKILTATVDNANVLLQIDNARLAADDFRTKYETELNLRMSVEADINGLRRVLDELTLARADLEMQIESLKEELAYLKKNHEEEMNALRGQVGGDVNVEMDAAPGVDLSRILNEMRDQYEKMAEKNRKDAEEWFFTKTEELNREVATNSELVQSGKSEISELRRTMQNLEIELQSQLSMKASLENSLEETKGRYCMQLAQIQEMIGSVEEQLAQLRCEMEQQNQEYKILLDVKTRLEQEIATYRRLLEGEDAHLSSSQFSSGSQSSRDVTSSSRQIRTKVMDVHDGKVVSTHEQVLRTKN

SEQ ID NO: 18 - DNA, 6307 bp, Artificial Sequence; identical to SEQ ID NO: 10
  Plasmid vector

SEQ ID NO: 19 - DNA, 6221 bp, Artificial Sequence; identical to SEQ ID NO: 11
  Plasmid vector

SEQ ID NO: 20 - DNA, 6269 bp, Artificial Sequence; identical to SEQ ID NO: 12
  Plasmid vector

SEQ ID NO: 21 - DNA, 6346 bp, Artificial Sequence; identical to SEQ ID NO: 13
  Plasmid vector

(19) United States
(12) Patent Application Publication
(10) Pub. No.: US 2010/0115636 A1
(43) Pub. Date: May 6, 2010

(54) PIG MODEL FOR BREAST CANCER, MITOCHONDRIA RELATED PROTEIN FOLDING DISORDERS AND/OR EPIDERMOLYSIS BULLOSA SIMPLEX

(75) Inventors: Peter Michael Kragh, Trondheim (NO); Lars Axel Bolund, Skodstrup (DK); Charlotte Brandt Sorensen, Hojbjerg (DK); Thomas Juhl Corydon, Risskov (DK); Niels Gregersen, Skodstrup (DK); Peter Bross, Arhus V (DK)

Correspondence Address: WEINGARTEN, SCHURGIN, GAGNEBIN & LEBOVICI LLP, TEN POST OFFICE SQUARE, BOSTON, MA, 02109, US

(73) Assignee: AARHUS UNIVERSITET, Århus C (DK)

(21) Appl. No.: 12/529,816

(22) PCT Filed: Mar. 6, 2008
(86) PCT No.: PCT/DK08/50057

§ 371 Date: Jan. 4, 2010

(30) Foreign Application Priority Data
Mar. 7, 2007 (DK) PA 2007 00341
Mar. 7, 2007 (DK) PA 2007 00342
Mar. 7, 2007 (DK) PA 2007 00344

(51) Int. Cl. G01N 33/00; A01K 67/00; C12N 15/00

(52) U.S. Cl. 800/3; 800/10; 800/21; 800/24

(57) ABSTRACT

The present invention relates to a genetically modified pig as a model for studying breast cancer, mitochondria related protein folding disorders and/or epidermolysis bullosa simplex. The modified pig model displays one or more phenotypes associated with any of said disorders. Disclosed is also a modified pig comprising a modified endogeneous BRCA1 and/or BRCA 2 gene, and/or a modified ornithine transcarbamylase gene, and/or a modified Keratin 14 gene and/or a transcriptional or translational product or part thereof. The invention further relates to methods for producing the modified pig; and methods for evaluating the effect of a therapeutical treatment of breast cancer, mitochondria related protein folding disorders and/or epidermolysis bullosa simplex; methods for screening the efficacy of a pharmaceutical composition; and a method for treatment of a human being suffering from breast cancer, mitochondria related protein folding disorders and/or epidermolysis bullosa simplex are disclosed.

### FIELD OF INVENTION

The present invention relates to a genetically modified pig as a model for studying breast cancer, mitochondria related protein folding disorders and/or epidermolysis bullosa simplex, wherein the pig model expresses at least one phenotype associated with said disease. The invention further relates to methods by which the genetically modified pig is produced. In addition, methods for evaluating the response of a therapeutical treatment of breast cancer, mitochondria related protein folding disorders and/or epidermolysis bullosa simplex, for screening the efficacy of a pharmaceutical composition, and a method for treatment of human being suffering from breast cancer, mitochondria related protein folding disorders and/or epidermolysis bullosa simplex are disclosed.

### BACKGROUND OF INVENTION

Transgenic, non-human animals can be used to understand the action of a single gene or genes in the context of the whole animal and the interrelated phenomena of gene activation, expression, and interaction. The technology has also led to the production of models for various diseases in humans and other animals which contributes significantly to an increased understanding of genetic mechanisms and of genes associated with specific diseases.

Traditionally, smaller animals such as mice have been used as disease models for human diseases and have been found to be suitable as models for certain diseases. However, their value as animal models for many human diseases is quite limited due to differences in mice compared to humans. Larger transgenic animals are much more suitable than mice for the study of many of the effects and treatments of most human diseases because of their greater similarity to humans in many aspects. Particularly, pigs are believed to be valuable as disease models for human diseases.

In one aspect, the present invention relates to breast cancer, which is the most prevalent disease and second leading cause of death among women in USA and Northern Europe. After lung cancer, it is the most fatal cancer in women, and the number of cases has significantly increased since the 1970s. Breast cancer is a cancer of the breast tissue. It is the most common form of cancer in females. Most cases of breast cancer are ‘sporadic’ not familial, and are caused by gene damage acquired to breast cells during the woman's lifetime (‘somatic’ mutations). A wide variety of genes is commonly mutated or incorrectly regulated in sporadic breast cancers and have been implicated in the development and progression of the disease. These include genes encoding growth factors and receptors, intracellular signaling molecules, cell cycle regulators, apoptosis (cell death) regulators, and adhesion molecules.

About 10% of the breast cancer incidents are of inherited origin. In general, cancers are considered to be a result of damage to DNA. How this mechanism occurs comes from several known or hypothesized factors. Some factors lead to an increased rate of mutation (exposure to estrogens) and decreased repair (the BRCA1, BRCA2 and p53 genes). Although many epidemiological risk factors, and biological co-factors and promoters have been identified, the majority of breast cancer incidence remains unexplained, and the primary cause is still unknown. In addition to the high penetrant genes, BRCA1 and BRCA2, contribution from inherited cancer syndromes as Li-Fraumeni (p53), Ataxia-telangiectasia (ATM), Cowden disease (PTEN), Peutz-Jeghers syndrome (LKB1/STK11) and mutations in CHK2 counts for 20-30% of the familiar cases.

The autosomal dominant genes, BRCA1 and BRCA2, have been linked to the rare familial form of breast cancer. People in families expressing mutations in these genes have a 60% to 80% risk of developing breast cancer according to Robbins Pathological Basis of Disease. If a mother or a sister was diagnosed breast cancer, the risk is about 2-fold higher than those women without a familial history. BRCA1 and BRCA2 are human tumor suppressor genes. BRCA1 regulates the cycle of cell division by keeping cells from growing and dividing too rapidly or in an uncontrolled way. In particular, it inhibits the growth of cells that line the milk ducts in the breast. The protein encoded by the BRCA1 gene is directly involved in the repair of damaged DNA. BRCA1 protein interacts with the protein encoded by the RAD51 gene to repair breaks in DNA. The BRCA2 protein, which has a function similar to that of BRCA1, also interacts with the RAD51 protein. By repairing DNA, these three proteins play a role in maintaining the stability of the human genome, and therefore are important suppressors of cancer development.

Hereditary breast cancer may thus be caused by mutations in BRCA1/2 genes. BRCA1 is involved in development of early onset breast and ovarian cancer in women, and BRCA2 is involved in development of early onset breast cancer in women and men. BRCA1 and BRCA2 proteins are of importance in DNA repair and maintenance of genome integrity.

A need exists for an efficient animal model which displays aspects that resemble human breast cancer. Such an animal model will allow for further studying the causes of breast cancer and to test drugs that will alleviate the symptoms in a large number of people suffering from breast cancer or even curing breast cancer.

Even though the genes responsible for inherited breast cancer or involved in the development of disease have been identified in humans it does not follow that animals transgenic for such mutations display a phenotype comparable to that of the human disease. However, the present invention has surprisingly shown that the genetically modified pig models according of the present invention display the breast cancer phenotype.

All proteins have to fold into specific three-dimensional structures for proper function. The protein structures, however, are not rigid. Instead, proteins have a dynamic life style, which may involve unfolding and refolding, complex association and dissociation. Protein misfolding can cause clinical disorders that are classified as “conformational diseases” due to the common features of their pathogenesis.

Several genetic disorders relate to protein folding defects: mutations in the cystic fibrosis transmembrane conductance regulator (CFTR) protein that lead to its misfolding cause cystic fibrosis, while folding deficient low-density lipoprotein (LDL) receptor protein variants cause familial hypercholesterolemia.

Mitochondrial dysfunction causes many diseases, and protein folding is essential for function of this organelle. For proteins to enter the mitochondria from the cytoplasm, they have to be unfolded in order to pass through the entry channels of the mitochondrial membranes. Therefore, once inside the mitochondria, these proteins have to fold into their native conformation for proper function.

Despite the differential clinical features of the various neurodegenerative disorders, the fact that neurons are highly dependent on oxidative energy metabolism has led to the suggestion that an underlying dysfunction in mitochondrial energy metabolism may result in neurodegeneration in general.

Mitochondria are involved in a number of important cellular functions. For example mitochondria play a key role in oxidative energy metabolism. Oxidative phosphorylation generates most of the cell's ATP, and any impairment of the organelle's ability to produce energy can have serious consequences. Moreover, deficient mitochondrial metabolism may generate reactive oxygen species, which is extremely deleterious for the cell. Therefore, mitochondrial dysfunction is likely to play a role in neuronal degeneration.

Ornithine transcarbamylase (OTC) localizes to mitochondria, and is normally expressed in the liver. OTC deficiency is the most common of the urea cycle disorders. The mutated enzyme results in impairment of the reaction that leads to condensation of carbamyl phosphate and ornithine to form citrulline. This impairment leads to reduced ammonia incorporation, which, in turn, causes symptomatic Hyperammonemia.

The central nervous system (CNS) is intolerant to free ammonia, and therefore, free ammonia is normally rapidly metabolized. Apparently, the CNS is particularly sensitive to the toxic effects of ammonia: many metabolic derangements occur as a consequence of high ammonia levels, including alteration of the metabolism of important compounds, such as pyruvate, lactate, glycogen, and glucose. As ammonia exceeds normal concentration, an increased disturbance of neurotransmission and synthesis of both gamma-aminobutyric acid receptor and glutamine occurs in the CNS. The mechanism for neurotoxicity of ammonia is not yet completely defined. However, the pathophysiology of hyperammonemia is the same as a CNS toxin that causes irritability, somnolence, vomiting, cerebral edema, and coma that leads to death.

In another aspect the present invention relates to mitochondria related protein folding disorders. Accumulation of misfolded proteins is the hallmark of a multitude of degenerative processes including neurodegenerative diseases, such as Alzheimer's disease, Parkinsons disease, and Huntingtons Chorea. It is generally believed that the accumulation of misfolded protein—through creation of cellular stress—is linked to the observed mitochondrial dysfunction and neuronal cell death. However, the relationship between the protein misfolding, which often occurs outside the mitochondria, and the mitochondrial dysfunction remains unclear.

“Huntington's disease” (also known as Huntington chorea) is used herein to refer to any inherited condition characterized by abnormal and/or uncontrolled body movements, mental and emotional problems, and loss of thinking ability (cognition).

The most common form of Huntington's disease is Adult-onset Huntington disease, which usually begins in middle age. Signs and symptoms can include irritability, depression, small involuntary movements, poor coordination, and trouble learning new information or making decisions. As the disease progresses, involuntary jerking movements (chorea) become more pronounced. Affected individuals may have trouble walking, speaking, and swallowing. People with the disorder also typically experience changes in personality and a decline in thinking and reasoning abilities. Individuals with this form of Huntington disease generally survive about 15 to 25 years after onset.

There is also an early-onset form of Huntington disease that begins in childhood or adolescence. Some of the clinical features of this disease differ from those of the adult-onset form. Signs and symptoms can include slowness, clumsiness, rigidity, loss of developmental milestones (such as motor skills), slow speech, and drooling. Seizures occur in 30 percent to 50 percent of individuals with this condition. The course of early-onset Huntington disease may be shorter than adult-onset Huntington disease; affected individuals generally survive 10 to 15 years after onset.

Huntington's disease is linked to the Huntingtin gene (HD gene, accession number: NM_{—}002111). The dysfunction and loss of nerve cells cause the signs and symptoms of Huntington disease.

“Parkinson's disease” is used herein to refer to an inherited condition usually associated with the following symptoms—all of which result from the loss of dopamine-producing brain cells: tremor or trembling of the arms, jaw, legs, and face; stiffness or rigidity of the limbs and trunk; bradykinesia—slowness of movement; postural instability, or impaired balance and coordination. The following genes are linked to Parkinson's disease: Alfa synuclein (SNCA, NM_{—}000345), Ubiquitin C-terminal hydrolase (UCHL1, NM_{—}004181), Leucine rich repeat kinase (LRRK2, NM_{—}198578).

Alzheimer's disease has been classified as a protein misfolding disease due to the accumulation of abnormally folded amyloid beta protein in the brains of Alzheimer's disease patients. Amyloid beta is a short peptide that is an abnormal proteolytic byproduct of the transmembrane protein amyloid precursor protein (APP), which seems to be involved in neuronal development. The presenilins are components of proteolytic complex involved in APP processing and degradation. Although amyloid beta monomers are soluble and harmless, they undergo a dramatic conformational change at sufficiently high concentration to form a beta sheet-rich tertiary structure that aggregates to form fibrils of amyloid, depositing outside neurons in dense formations. Abnormal aggregation of the tau protein is thought also to be involved in Alzheimer's disease as hyperphosphorylated tau accumulated and aggregates into masses inside nerve cell bodies known as neurofibrillary tangles.

“Alzheimer's disease” is used herein to refer to any neurodegenerative brain disorder characterized by progressive memory loss and severe dementia in advanced cases. Alzheimer's disease is associated with certain abnormalities in brain tissue, involving a particular protein, beta-amyloid. Memory impairment is a necessary feature for the diagnosis of this type of dementia. Change in one of the following areas must also be present: language, decision-making ability, judgment, attention, and other areas of mental function and personality.

The rate of progression is different for each person. If Alzheimer's disease develops rapidly, it is likely to continue to progress rapidly. If it has been slow to progress, it will likely continue on a slow course. There are two types of Alzheimer's disease—early onset and late onset. In early onset Alzheimer's disease, symptoms first appear before age 60. Early onset Alzheimer's disease is much less common, accounting for only 5-10% of cases. However, it tends to progress rapidly.

Early onset disease can run in families and involves autosomal dominant, inherited mutations that may be the cause of the disease. So far, three early onset genes have been identified. Late onset Alzheimer's disease, the most common form of the disease, develops in people 60 and older and is thought to be less likely to occur in families. Late onset Alzheimer's disease may run in some families, but the role of genes is less direct and definitive. These genes may not cause the problem itself, but simply increase the likelihood of formation of plaques and tangles or other Alzheimer's disease-related pathologies in the brain.

The cause of Alzheimer's disease is not entirely known but is thought to include both genetic and environmental factors. A diagnosis of Alzheimer's disease is made based on characteristic symptoms and by excluding other causes of dementia. The only way to validate a case of Alzheimer's disease is by microscopic examination of a sample of brain tissue after death.

The brain tissue shows “neurofibrillary tangles”, “neuritic plaques” (abnormal clusters of dead and dying nerve cells, other brain cells, and protein), and “senile plaques” (areas where products of dying nerve cells have accumulated around protein). Although these changes occur to some extent in all brains with age, there are many more of them in the brains of people with Alzheimer's disease.

The destruction of nerve cells (neurons) leads to a decrease in neurotransmitters (substances secreted by a neuron to send a message to another neuron). The correct balance of neurotransmitters is critical to the brain. By causing both structural and chemical problems in the brain, Alzheimer's disease appears to disconnect areas of the brain that normally work together.

Existing animal models, display only a few aspects that resembles human diseases due to mitochondria related protein folding disorders. Thus, a need exists for an efficient animal model which displays aspects that resemble human mitochondria related protein folding disorders. Such an animal model will allow for further studying the causes of mitochondria related protein folding disorders and to test drugs that will alleviate the symptoms of a large number of people suffering from mitochondria related protein folding disorders.

Even though the gene responsible for mitochondria related protein folding disorders or involved in the development of disease have been identified in humans it does not follow that animals transgenic for such mutations display a phenotype comparable to that of the human disease. However, the present invention has surprisingly shown that the genetically modified pig models according of the present invention display the mitochondria related protein folding disorders phenotype.

In yet another aspect, the present invention relates to a pig model for epidermolysis bullosa simplex. Epidermolysis bullosa is a group of inherited disorders in which the skin blisters very easily. The skin is so fragile in people with epidermolysis bullosa that even minor rubbing may cause blistering. At times, the person may not be aware of rubbing or injuring the skin even though blisters develop. In severe epidermolysis bullosa, blisters are not confined to the outer skin. They may develop inside the body, in such places as the linings of the mouth, esophagus, stomach, intestines, upper airway, bladder, and the genitals. Most forms of epidermolysis bullosa are evident at birth. Other signs may include thickened skin on the palms of the hands and soles of the feet; rough, thickened, or absent fingernails or toenails. Less common signs include growth retardation; anemia (a reduction in the red blood cells that carry oxygen to all parts of the body); scarring of the skin; and milia, which are small white skin cysts. This disorder can be both disabling and disfiguring, and some forms may lead to early death. The disease results when skin layers separate after minor trauma. Defects of several proteins within the skin are at fault.

Three types of Epidermolysis Bullosa are known, each characterized as a distinct disorder. Patients suffering from with Epidermolysis Bullosa simplex cannot develop one of the other forms Dystrophic Epidermolysis Bullosa or Junctional Epidermolysis Bullosa.

Epidermolysis Bullosa Simplex is usually inherited as an autosomal dominant disease, characterized by the presence of extremely fragile skin and recurrent blister formation The genes responsible for the disease are those that provide instructions for producing keratin, a fibrous protein in the top layer of skin. As a result, the skin splits in the epidermis, producing a blister. The condition typically begins with blistering that is evident at birth or shortly afterward. There are three main types of Epidermolysis Bullosa Simplex Weber Cockayne, Köbner, and Dowling Meara Epidermolysis Bullosa Simplex. Weber Cockayne is the most common type of Epidermolysis Bullosa Simplex and is a relatively mild form, in which blisters rarely extend beyond the feet and hands. Blisters may not become evident until the child begins to walk. In Kobner Epidermolysis Bullosa Simplex, blistering may be obvious from birth, or develop during the first few weeks of life. Sites of blistering respond to areas where friction is caused by clothing and frequently appear around the edges of the nappy. Blisters are often seen inside the mouth but do not generally cause a problem during feeding. Dowling Meara is the most severe form of Epidermolysis Bullosa Simplex and blistering appears already during or shortly after birth. Blisters may develop in cluster, and spread like rings.

Treatment of the blisters and wound can be very time consuming and interfere with the patients normal life, such as the ability to attend school or go to work. Currently no cure exists for patients suffering from Epidermolysis Bullosa. The current treatment of the symptoms include taking care of the blisters and wounds, and reducing the risk of new blister forming as well as the risk of infection in the many wounds that develop.

Thus, a need exists for an efficient animal model which displays aspects that resemble human epidermolysis bullosa simplex. Such an animal model will allow for further studying the causes of epidermolysis bullosa simplex and to test drugs that will cure the disease or alleviate the symptoms of a large number of people suffering from epidermolysis bullosa simplex.

The genes responsible for Epidermis bullosa simplex have been identified in humans. Even though causative mutations in genes have been identified in humans as being involved in the development of particular diseases in humans it does not follow that animals transgenic for such mutations display a phenotype comparable to that of the human disease. However, the present invention has surprisingly shown that the genetically modified pig models according of the present invention display the epidermis bullosa simplex phenotype.

### SUMMARY OF INVENTION

### Breast Cancer

The present invention concerns a genetically modified pig model, which allows for the study of breast cancer. Thus, one aspect of the present invention relates to a genetically modified pig as a model for studying breast cancer, wherein the pig model expresses at least one phenotype associated with said disease and/or a modified pig comprising at least one modified endogeneous

i) exon 3 or part thereof of a BRCA1 gene and/or
ii) porcine BRCA1 gene or part thereof comprising a nucleotide substitution from T to G resulting in amino acid substitution from Cys to Gly at codon 61 of exon 3 and/or
iii) exon 11 or part thereof of the BRCA1 gene and/or
iv) porcine BRCA1 gene or part thereof comprising a deletion of at least one allele of exon 11 or part thereof of the BRCA1 gene and/or
v) exon 11 or part thereof of the BRCA2 gene, and/or
vi) porcine BRCA2 gene comprising a deletion of at least one allele of exon 11 or part thereof of the BRCA2 gene and/or
a transcriptional and/or translational product or part thereof.

Embodiments for the present invention comprises, mini-pigs for example selected from the group consisting of Goettingen, Yucatan, Bama Xiang Zhu, Wuzhishan and Xi Shuang Banna, including any combination thereof. However, another embodiment relates to pigs that are not a mini-pig, such as the species of Sus domesticus, for example where the pig is selected from the group consisting of Landrace, Yorkshire, Hampshire, Duroc, Chinese Meishan, Berkshire and Piêtrain, including any combination thereof.

Embodiments of the present invention comprise the genetically modified pig, wherein the pig is transgenic due to at least one mutation in exon 3 or part thereof of the BRCA1 gene, and/or due to a nucleotide substitution from T to G resulting in amino acid substitution from Cys to Gly at codon 61 of exon 3, and/or due to at least one mutation in exon 11 or part thereof of the BRCA2 gene, and/or due to deletion of at least one allele of exon 11 or part thereof of the BRCA 2 gene, and/or due to deletion of SEQ ID NO: 2 or part thereof, and/or due to at least one mutation in exon 11 or part thereof of the BRCA 1 gene, and/or due to deletion of at least one allele of exon 11 or part thereof of the BRCA 1 gene, and/or due to deletion is a deletion of SEQ ID NO: 3 or part thereof, and/or due to at least one mutation in exon 3 or part thereof of the BRCA1 gene, at least one mutation in exon 11 or part thereof of the BRCA 1 gene and at least one mutation in exon 11 or part thereof of the BRCA 2 gene.

A second aspect of the present invention relates to a method for producing a transgenic pig, porcine blastocyst, embryo, fetus and/or donor cell as a model for breast cancer comprising:

i) establishing at least one oocyte
ii) separating the oocyte into at least three parts obtaining at least one cytoplast,
iii) establishing a donor cell or cell nucleus having desired genetic properties,
iv) fusing at least one cytoplast with the donor cell or membrane surrounded cell nucleus,
v) obtaining a reconstructed embryo,
vi) activating the reconstructed embryo to form an embryo; culturing said embryo; and
vii) transferring said cultured embryo to a host mammal such that the embryo develops into a genetically modified fetus,
wherein said transgenic embryo comprises steps i) to v) and/or vi),
wherein said transgenic blastocyst comprises steps i) to vi) and/or vii),
wherein said transgenic fetus comprises steps i) to vii)

A third aspect of the present invention pertains to a genetically modified porcine blastocyst derived from the genetically modified pig model as defined in the present invention and/or a modified porcine blastocyst comprising at least one modified endogeneous

i) exon 3 or part thereof of a BRCA1 gene and/or
ii) porcine BRCA1 gene or part thereof comprising a nucleotide substitution from T to G resulting in amino acid substitution from Cys to Gly at codon 61 of exon 3 and/or
iii) exon 11 or part thereof of the BRCA1 gene and/or
iv) porcine BRCA1 gene or part thereof comprising a deletion of at least one allele of exon 11 or part thereof of the BRCA1 gene and/or
v) exon 11 or part thereof of the BRCA2 gene, and/or
vi) porcine BRCA2 gene comprising a deletion of at least one allele of exon 11 or part thereof of the BRCA2 gene and/or
a transcriptional and/or translational product or part thereof.

A fourth aspect of the present invention relates to a genetically modified porcine embryo derived from the genetically modified pig model as defined in the present invention and/or a modified porcine embryo comprising at least one modified endogeneous

i) exon 3 or part thereof of a BRCA1 gene and/or
ii) porcine BRCA1 gene or part thereof comprising a nucleotide substitution from T to G resulting in amino acid substitution from Cys to Gly at codon 61 of exon 3 and/or
iii) exon 11 or part thereof of the BRCA1 gene and/or
iv) porcine BRCA1 gene or part thereof comprising a deletion of at least one allele of exon 11 or part thereof of the BRCA1 gene and/or
v) exon 11 or part thereof of the BRCA2 gene, and/or
vi) porcine BRCA2 gene comprising a deletion of at least one allele of exon 11 or part thereof of the BRCA2 gene and/or
a transcriptional and/or translational product or part thereof.

A fifth aspect relates to a genetically modified porcine fetus derived from the genetically modified pig model as defined in the present invention and/or a modified porcine fetus comprising at least one modified pig model as defined in claim 1 and/or

a modified porcine fetus comprising at least one modified endogeneous
i) exon 3 or part thereof of a BRCA1 gene and/or
ii) porcine BRCA1 gene or part thereof comprising a nucleotide substitution from T to G resulting in amino acid substitution from Cys to Gly at codon 61 of exon 3 and/or
iii) exon 11 or part thereof of the BRCA1 gene and/or
iv) porcine BRCA1 gene or part thereof comprising a deletion of at least one allele of exon 11 or part thereof of the BRCA1 gene and/or
v) exon 11 or part thereof of the BRCA2 gene, and/or
vi) porcine BRCA2 gene comprising a deletion of at least one allele of exon 11 or part thereof of the BRCA2 gene and/or
a transcriptional and/or translational product or part thereof.

A sixth aspect relates to a genetically modified porcine fetus derived from the genetically modified pig model as defined in the present invention, and/or

a modified porcine fetus comprising at least one modified
i) exon 3 or part thereof of the BRCA1 gene and/or
ii) porcine BRCA1 comprising a nucleotide substitution from T to G resulting in amino acid substitution from Cys to Gly at codon 61 of exon 3 and/or
iii) exon 11 or part thereof of the BRCA1 gene and/or
iv) porcine BRCA1 gene comprising a deletion of at least one allele of exon 11 or part thereof of the BRCA1 gene and/or
v) exon 11 or part thereof of the BRCA2 gene, and/or
vi) porcine BRCA2 gene comprising a deletion of at least one allele of exon 11 or part thereof of the BRCA2 gene and/or
a transcriptional and/or translational product thereof.

A seventh aspect relates to a genetically modified porcine donor cell and/or cell nucleus derived from the genetically modified pig model as defined in the present invention and/or a modified porcine donor cell and/or cell nucleus comprising at least one modified endogeneous

i) exon 3 or part thereof of a BRCA1 gene and/or
ii) porcine BRCA1 gene or part thereof comprising a nucleotide substitution from T to G resulting in amino acid substitution from Cys to Gly at codon 61 of exon 3 and/or
iii) exon 11 or part thereof of the BRCA1 gene and/or
iv) porcine BRCA1 gene or part thereof comprising a deletion of at least one allele of exon 11 or part thereof of the BRCA1 gene and/or
v) exon 11 or part thereof of the BRCA2 gene, and/or
vi) porcine BRCA2 gene comprising a deletion of at least one allele of exon 11 or part thereof of the BRCA2 gene and/or
a transcriptional and/or translational product or part thereof.

A eighth aspect relates to a method for producing a transgenic pig as a model for breast cancer comprising:

i) establishing at least one oocyte
ii) separating the oocyte into at least three parts obtaining at least one cytoplast,
iii) establishing a donor cell or cell nucleus having desired genetic properties,
iv) fusing at least one cytoplast with the donor cell or membrane surrounded cell nucleus,
v) obtaining a reconstructed embryo,
vi) activating the reconstructed embryo to form an embryo; culturing said embryo; and
vii) transferring said cultured embryo to a host mammal such that the embryo develops into a genetically modified foetus.

Embodiments of the second to eighth aspects comprise one or more of the features as defined elsewhere herein, wherein the method for activation of the reconstructed embryo is selected from the group of methods consisting of electric pulse, chemically induced shock, increasing intracellular levels of divalent cations and reducing phosphorylation. Further embodiments of the second and third aspects comprise one or more of the features as defined above, wherein steps iv) and vi) are performed sequentially or simultaneously, and embodiments comprising one or more of the features, wherein the embryo is cultured in vitro. Such embryo may be cultured in sequential culture. The embryo, for example at the blastocyst stage, is cryopreserved prior to transfer to a host mammal.

For the methods of the present invention embodiments cover pigs, mini-pigs for example selected from the group consisting of Goettingen, Yucatan, Bama Xiang Zhu, Wuzhishan and Xi Shuang Banna, including any combination thereof. However, another embodiment relates to pigs that are not a mini-pig, such as the species of Sus domesticus, for example where the pig is selected from the group consisting of Landrace, Yorkshire, Hampshire, Duroc, Chinese Meishan, Berkshire and Piêtrain, including any combination thereof.

A ninth aspect pertains to a method for evaluating the response of a therapeutical treatment of breast cancer, said method comprising the steps of

i) providing the pig model according to the present invention,
ii) treating said pig model with a pharmaceutical composition exerting an effect on said phenotype, and
iii) evaluating the effect observed.

A tenth aspect relates to a method for screening the efficacy of a pharmaceutical composition, said method comprising the steps of

i) providing the pig model according to the present invention,
ii) expressing in said pig model said genetic determinant and exerting said phenotype for said disease,
iii) administering to said pig model a pharmaceutical composition the efficacy of which is to be evaluated, and
iv) evaluating the effect, if any, of the pharmaceutical composition on the phenotype exerted by the genetic determinant when expressed in the pig model.

A eleventh aspect relates to a method for screening the efficacy of a pharmaceutical composition, said method comprising the steps of

i) providing the pig model according to the present invention,
ii) expressing in said pig model said genetic determinant and exerting said phenotype for said disease,
iii) administering to said pig model a pharmaceutical composition the efficacy of which is to be evaluated, and
iv) evaluating the effect, if any, of the pharmaceutical composition on the phenotype exerted by the genetic determinant when expressed in the pig model.

A eleventh aspect relates to a method for treatment of a human being suffering from breast cancer, said method comprising the initial steps of

i) providing the pig model according to the present invention 1 to 20,
ii) expressing in said pig model said genetic determinant and exerting said phenotype for said disease,
iii) administering to said pig model a pharmaceutical composition the efficacy of which is to be evaluated, and
iv) evaluating the effect observed, and
v) treating said human being suffering from breast cancer based on the effects observed in the pig model.

Mitochondria Related Protein Folding Disorders

The present invention concerns a genetically modified pig model which allows for the study of mitochondria related protein folding disorders.

Thus, a twelfth aspect of the present invention relates to a genetically modified pig as a genetically modified pig as a model for studying mitochondria related protein folding disorders, wherein the pig model expresses at least one phenotype associated with said disease and/or a modified pig comprising at least one modified

i) rat Ornithine TransCarbamylase (OTC) gene or part thereof, and/or
ii) human Ornithine TransCarbamylase gene or part thereof, and/or
iii) porcine Ornithine TransCarbamylase gene or part thereof, and/or
iv) rat Ornithine TransCarbamylase cDNA or part thereof, and/or
v) porcine Ornithine TransCarbamylase cDNA or part thereof, and/or
vi) human Ornithine TransCarbamylase cDNA or part thereof, and/or a transcriptional and/or translational product or part thereof.

Embodiments for the present invention comprises, mini-pigs for example selected from the group consisting of Goettingen, Yucatan, Bama Xiang Zhu, Wuzhishan and Xi Shuang Banna, including any combination thereof. However, another embodiment relates to pigs that are not a mini-pig, such as the species of Sus domesticus, for example where the pig is selected from the group consisting of Landrace, Yorkshire, Hampshire, Duroc, Chinese Meishan, Berkshire and Piêtrain, including any combination thereof.

Embodiments of the present invention comprise the genetically modified pig, wherein the pig is transgenic due to insertion of at least a modified rat Ornithine TransCarbamylase (OTC) gene or part thereof, and/or due to insertion of at least a human Ornithine TransCarbamylase (OTC) or part thereof, and/or due to insertion of at least a porcine Ornithine TransCarbamylase (OTC) or part thereof, and/or due to insertion of at least a porcine, human and/or rat Ornithine TransCarbamylase (OTC) gene or part thereof, which is modified by lacking a carbamyl phosphate-binding domain, and/or due to insertion of at least a rat Ornithine TransCarbamylase cDNA or part thereof, and/or due to insertion of at least a porcine Ornithine TransCarbamylase cDNA or part thereof, and/or due to insertion of at least a human Ornithine TransCarbamylase cDNA or part thereof, and/or due to insertion of at least a porcine, human and/or rat Ornithine TransCarbamylase (OTC) cDNA or part thereof, which is modified by lacking a carbamyl phosphate-binding domain.

A thirteenth aspect of the present invention relates to a method for producing a transgenic pig, porcine blastocyst, embryo, fetus and/or donor cell as a model for mitochondria related protein folding disorders comprising:

i) establishing at least one oocyte
ii) separating the oocyte into at least three parts obtaining at least one cytoplast,
iii) establishing a donor cell or cell nucleus having desired genetic properties,
iv) fusing at least one cytoplast with the donor cell or membrane surrounded cell nucleus,
v) obtaining a reconstructed embryo,
vi) activating the reconstructed embryo to form an embryo; culturing said embryo; and
vii) transferring said cultured embryo to a host mammal such that the embryo develops into a genetically modified fetus,
wherein said transgenic embryo comprises steps i) to v) and/or vi),
wherein said transgenic blastocyst comprises steps i) to vi) and/or vii),
wherein said transgenic fetus comprises steps i) to vii)

A fourteenth aspect of the present invention relates to a genetically modified porcine blastocyst derived from the genetically modified pig model as defined in the present invention and/or

a modified porcine blastocyst comprising at least one modified
i) rat Ornithine TransCarbamylase (OTC) gene or part thereof, and/or
ii) human Ornithine TransCarbamylase gene or part thereof, and/or
iii) porcine Ornithine TransCarbamylase gene or part thereof, and/or
iv) rat Ornithine TransCarbamylase cDNA or part thereof, and/or
v) porcine Ornithine TransCarbamylase cDNA or part thereof, and/or
vi) human Ornithine TransCarbamylase cDNA or part thereof, and/or
a transcriptional and/or translational product thereof.

A fifteenth aspect of the present invention pertains to a genetically modified porcine embryo derived from the genetically modified pig model as defined in the present invention and/or a modified porcine embryo comprising at least one modified

i) rat Ornithine TransCarbamylase (OTC) gene or part thereof, and/or
ii) human Ornithine TransCarbamylase gene or part thereof, and/or
iii) porcine Ornithine TransCarbamylase gene or part thereof, and/or
iv) rat Ornithine TransCarbamylase cDNA or part thereof, and/or
v) porcine Ornithine TransCarbamylase cDNA or part thereof, and/or
vi) human Ornithine TransCarbamylase cDNA or part thereof, and/or
a transcriptional and/or translational product thereof.

A sixteenth aspect of the present invention relates to a genetically modified porcine fetus derived from the genetically modified pig model as defined in the present invention and/or a modified porcine fetus comprising at least one modified

i) rat Ornithine TransCarbamylase (OTC) gene or part thereof, and/or
ii) human Ornithine TransCarbamylase gene or part thereof, and/or
iii) porcine Ornithine TransCarbamylase gene or part thereof, and/or
iv) rat Ornithine TransCarbamylase cDNA or part thereof, and/or
v) porcine Ornithine TransCarbamylase cDNA or part thereof, and/or
vi) human Ornithine TransCarbamylase cDNA or part thereof, and/or
a transcriptional and/or translational product thereof.

A seventeenth aspect of the present invention relates to a A genetically modified porcine donor cell and/or cell nucleus derived from the genetically modified pig model as defined in the present invention and/or a modified porcine donor cell and/or cell nucleus comprising at least one modified

i) rat Ornithine TransCarbamylase (OTC) gene or part thereof, and/or
ii) human Ornithine TransCarbamylase gene or part thereof, and/or
iii) porcine Ornithine TransCarbamylase gene or part thereof, and/or
iv) rat Ornithine TransCarbamylase cDNA or part thereof, and/or
v) porcine Ornithine TransCarbamylase cDNA or part thereof, and/or
vi) human Ornithine TransCarbamylase cDNA or part thereof, and/or
a transcriptional and/or translational product thereof.

Embodiments of the thirteenth to seventeenth aspects comprise one or more of the features as defined in any of the preceding claims, wherein the method for activation of the reconstructed embryo is selected from the group of methods consisting of electric pulse, chemically induced shock, increasing intracellular levels of divalent cations and reducing phosphorylation. Further embodiments of the second and third aspects comprise one or more of the features as defined above, wherein steps iv) and vi) are performed sequentially or simultaneously, and embodiments comprising one or more of the features, wherein the embryo is cultured in vitro. Such embryo may be cultured in sequential culture. The embryo, for example at the blastocyst stage, is cryopreserved prior to transfer to a host mammal.

For the methods of the present invention embodiments cover pigs, mini-pigs for example selected from the group consisting of Goettingen, Yucatan, Bama Xiang Zhu, Wuzhishan and Xi Shuang Banna, including any combination thereof. However, another embodiment relates to pigs that are not a mini-pig, such as the species of Sus domesticus, for example where the pig is selected from the group consisting of Landrace, Yorkshire, Hampshire, Duroc, Chinese Meishan, Berkshire and Piêtrain, including any combination thereof.

A eighteenth aspect of the present invention relates to a method for evaluating the effect of a therapeutical treatment of mitochondria related protein folding disorders, said method comprising the steps of

i) providing the pig model according to the present invention,
ii) treating said pig model with a pharmaceutical composition exerting an effect on said phenotype, and
iii) evaluating the effect observed.

An nineteenth aspect of the present invention relates to a method for screening the efficacy of a pharmaceutical composition, said method comprising the steps of

i) providing the pig model according to the present invention,
ii) expressing in said pig model said genetic determinant and exerting said phenotype for said disease,
iii) administering to said pig model a pharmaceutical composition the efficacy of which is to be evaluated, and
iv) evaluating the effect, if any, of the pharmaceutical composition on the phenotype exerted by the genetic determinant when expressed in the pig model.

A twentieth aspect of the present invention relates to a method for treatment of a human being suffering from mitochondria related protein folding disorders, said method comprising the initial steps of

i) providing the pig model according to the present invention,
ii) expressing in said pig model said genetic determinant and exerting said phenotype for said disease,
iii) administering to said pig model a pharmaceutical composition the efficacy of which is to be evaluated, and
iv) evaluating the effect observed, and
v) treating said human being suffering from mitochondria related protein folding disorders based on the effects observed in the pig model.

### Epidermolysis Bullosa Simplex

The present invention concerns a genetically modified pig model which allows for the study of Epidermis bullosa simplex.

Thus, a twenty-first aspect of the present invention relates to a genetically modified pig as a model for studying epidermolysis bullosa simplex, wherein the pig model expresses at least one phenotype associated with said disease and/or a modified pig comprising at least one modified

i) porcine keratin 14 gene or part thereof, and/or
ii) human keratin 14 gene or part thereof, and/or
iii) porcine keratin 14 cDNA or part thereof, and/or
iv) human keratin 14 cDNA or part thereof, and/or
a transcriptional and/or translational product or part thereof.

Embodiments for the present invention comprises, mini-pigs for example selected from the group consisting of Goettingen, Yucatan, Bama Xiang Zhu, Wuzhishan and Xi Shuang Banna, including any combination thereof. However, another embodiment relates to pigs that are not a mini-pig, such as the species of Sus domesticus, for example where the pig is selected from the group consisting of Landrace, Yorkshire, Hampshire, Duroc, Chinese Meishan, Berkshire and Piêtrain, including any combination thereof.

Embodiments of the present invention comprise the genetically modified pig, wherein the pig is transgenic due to insertion of at least a modified porcine keratin 14 gene or part thereof, or due to insertion of at least a modified human keratin 14 gene or part thereof, or due to insertion of at least a modified human keratin 14 cDNA or part thereof, or due to insertion of at least a modified porcine keratin 14 cDNA or part thereof.

A twenty-second aspect of the present invention relates to a method for producing a transgenic pig, porcine blastocyst, embryo, fetus and/or donor cell as a model for epidermolysis bullosa simplex comprising:

i) establishing at least one oocyte
ii) separating the oocyte into at least three parts obtaining at least one cytoplast,
iii) establishing a donor cell or cell nucleus having desired genetic properties,
iv) fusing at least one cytoplast with the donor cell or membrane surrounded cell nucleus,
v) obtaining a reconstructed embryo,
vi) activating the reconstructed embryo to form an embryo; culturing said embryo; and
vii) transferring said cultured embryo to a host mammal such that the embryo develops into a genetically modified fetus,
wherein said transgenic embryo comprises steps i) to v) and/or vi),
wherein said transgenic blastocyst comprises steps i) to vi) and/or vii),
wherein said transgenic fetus comprises steps i) to vii).

A twenty-third aspect of the present invention relates to a genetically modified porcine blastocyst derived from the genetically modified pig model as defined in the present invention and/or a modified porcine blastocyst comprising at least one modified

i) porcine keratin 14 gene or part thereof, and/or
ii) human keratin 14 gene or part thereof, and/or
iii) porcine keratin 14 cDNA or part thereof, and/or
iv) human keratin 14 cDNA or part thereof, and/or
a transcriptional and/or translational product or part thereof.

A twenty-fourth aspect of the present invention relates to a genetically modified porcine embryo derived from the genetically modified pig model as defined in the present invention and/or a modified porcine embryo comprising at least one modified

i) porcine keratin 14 gene or part thereof, and/or
ii) human keratin 14 gene or part thereof, and/or
iii) porcine keratin 14 cDNA or part thereof, and/or
iv) human keratin 14 cDNA or part thereof, and/or
a transcriptional and/or translational product or part thereof.

A twenty-fifth aspect of the present invention relates to a genetically modified porcine fetus derived from the genetically modified pig model as defined in the present invention and/or a modified porcine fetus comprising at least one modified

i) porcine keratin 14 gene or part thereof, and/or
ii) human keratin 14 gene or part thereof, and/or
iii) porcine keratin 14 cDNA or part thereof, and/or
iv) human keratin 14 cDNA or part thereof, and/or
a transcriptional and/or translational product or part thereof.

A twenty-sixth aspect of the present invention relates to a genetically modified porcine donor cell and/or cell nucleus derived from the genetically modified pig model as defined in the present invention and/or a modified porcine donor cell and/or cell nucleus comprising at least one modified

i) porcine keratin 14 gene or part thereof, and/or
ii) human keratin 14 gene or part thereof, and/or
iii) porcine keratin 14 cDNA or part thereof, and/or
iv) human keratin 14 cDNA or part thereof, and/or
a transcriptional and/or translational product or part thereof.

Embodiments of the twenty-second to twenty-sixth aspects comprise one or more of the features as defined in any of the preceding claims, wherein the method for activation of the reconstructed embryo is selected from the group of methods consisting of electric pulse, chemically induced shock, increasing intracellular levels of divalent cations and reducing phosphorylation. Further embodiments of the second and third aspects comprise one or more of the features as defined above, wherein steps iv) and vi) are performed sequentially or simultaneously, and embodiments comprising one or more of the features, wherein the embryo is cultured in vitro. Such embryo may be cultured in sequential culture. The embryo, for example at the blastocyst stage, is cryopreserved prior to transfer to a host mammal.

For the methods of the present invention embodiments cover pigs, mini-pigs for example selected from the group consisting of Goettingen, Yucatan, Bama Xiang Zhu, Wuzhishan and Xi Shuang Banna, including any combination thereof. However, another embodiment relates to pigs that are not a mini-pig, such as the species of Sus domesticus, for example where the pig is selected from the group consisting of Landrace, Yorkshire, Hampshire, Duroc, Chinese Meishan, Berkshire and Piêtrain, including any combination thereof.

A twenty-seventh aspect of the present invention relates to a method for evaluating the effect of a therapeutical treatment of epidermolysis bullosa simplex, said method comprising the steps of

i) providing the pig model according to the present invention,
ii) treating said pig model with a pharmaceutical composition exerting an effect on said phenotype, and
iii) evaluating the effect observed.

A twenty-eighth aspect of the present invention relates to a method for screening the efficacy of a pharmaceutical composition, said method comprising the steps of

i) providing the pig model according to the present invention,
ii) expressing in said pig model said genetic determinant and exerting said phenotype for said disease,
iii) administering to said pig model a pharmaceutical composition the efficacy of which is to be evaluated, and
iv) evaluating the effect, if any, of the pharmaceutical composition on the phenotype exerted by the genetic determinant when expressed in the pig model.

A twenty-ninth aspect of the present invention relates to a method for treatment of a human being suffering from epidermolysis bullosa simplex, said method comprising the initial steps of

i) providing the pig model according to the present invention,
ii) expressing in said pig model said genetic determinant and exerting said phenotype for said disease,
iii) administering to said pig model a pharmaceutical composition the efficacy of which is to be evaluated, and
iv) evaluating the effect observed, and
v) treating said human being suffering from epidermolysis bullosa simplex based on the effects observed in the pig model.

### DESCRIPTION OF DRAWINGS

### Breast Cancer

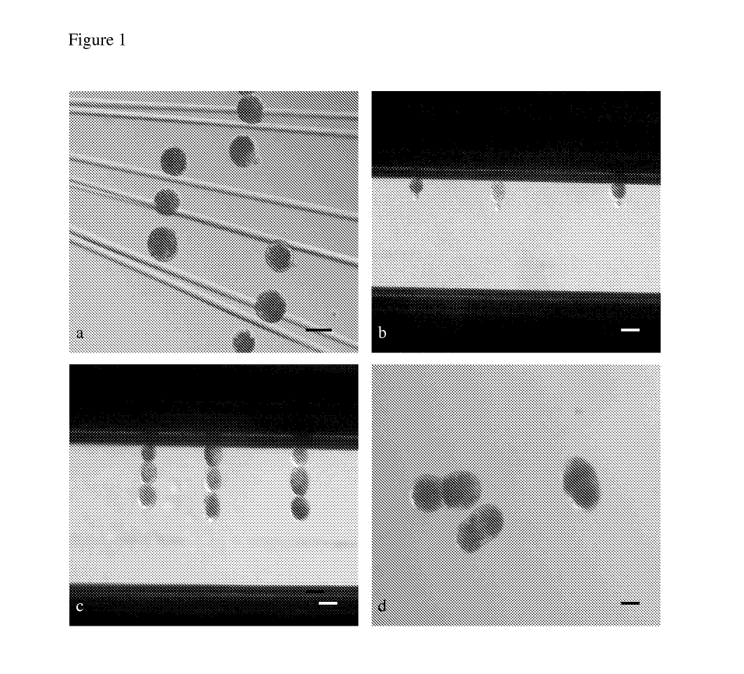

FIG. 1. (a) Oocytes trisection; (b) couplets of fibroblast-oocyte fragment for the first fusion; (c) embryos reconstructed with triplets (note elongation under the AC currency); (d) triplets fusion. Scale bar=50 μm.

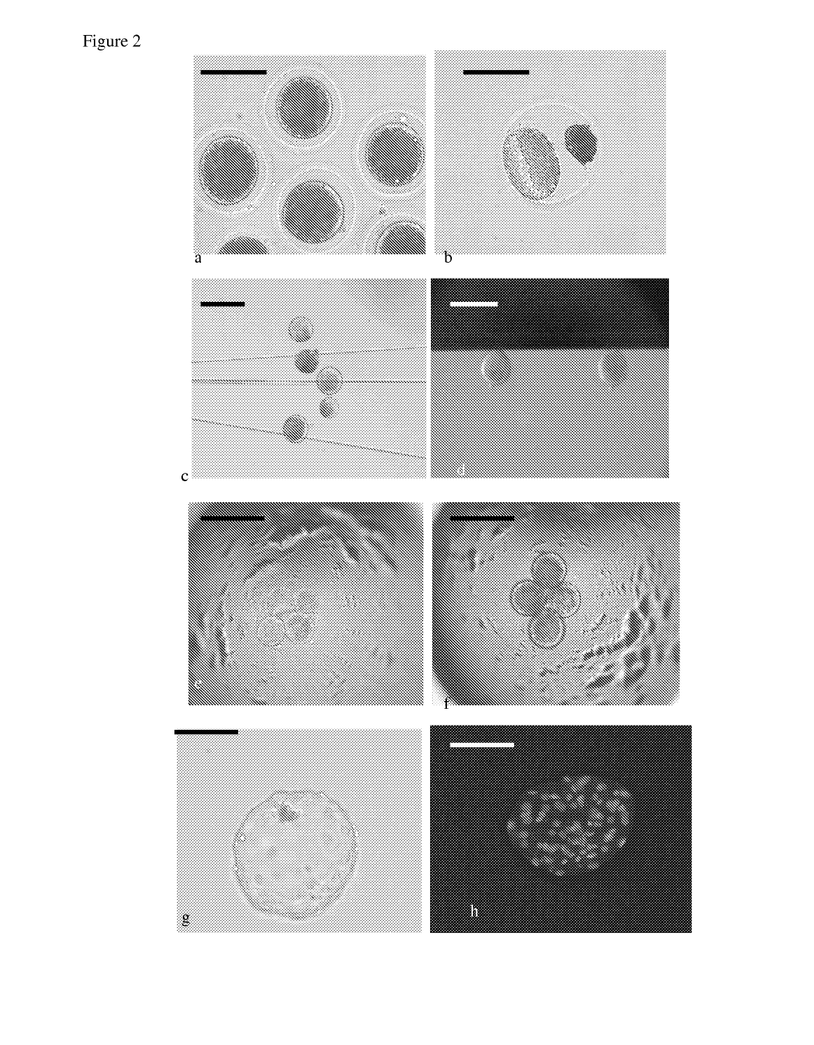

FIG. 2. (a) In vitro matured oocytes after partial zona digestion. (b) Delipated oocytes after centrifugation. (c) Bisection of delipated oocytes. (d) Couplets of fibroblast-oocyte fragment for the first fusion. (e) Four-cell stage reconstructed embryos developed from delipated oocytes. (f) Four-cell stage reconstructed embryos developed from intact oocytes. (g) Re-expanded blastocysts from delipated embryos after warming. (h) Hoechst staining and UV illumination of re-expanded blastocysts from delipated embryos after warming. Bar represents 100 μm.

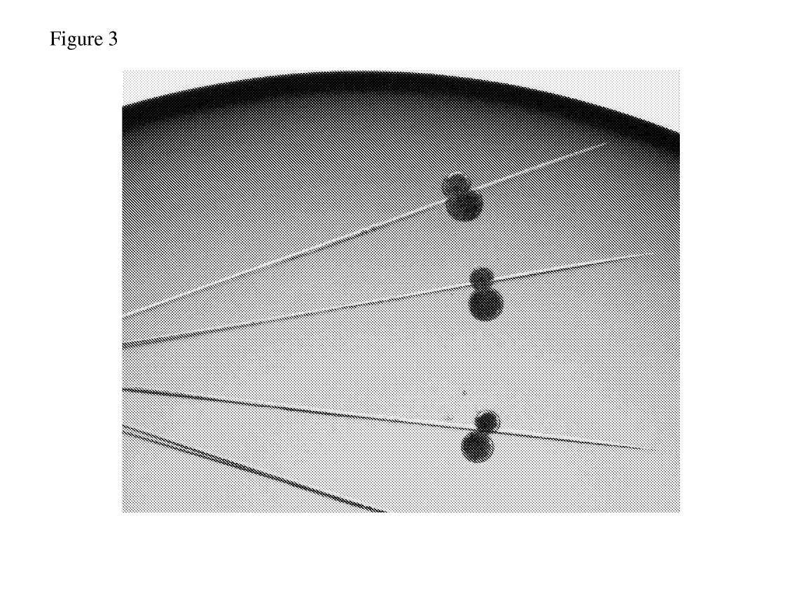

FIG. 3. Bisection at chemically assisted enucleation. Note the extrusion cone or polar body connected to the smaller part (putative karyoplast). Stereomicroscopic picture. Bar represents 50 μm.

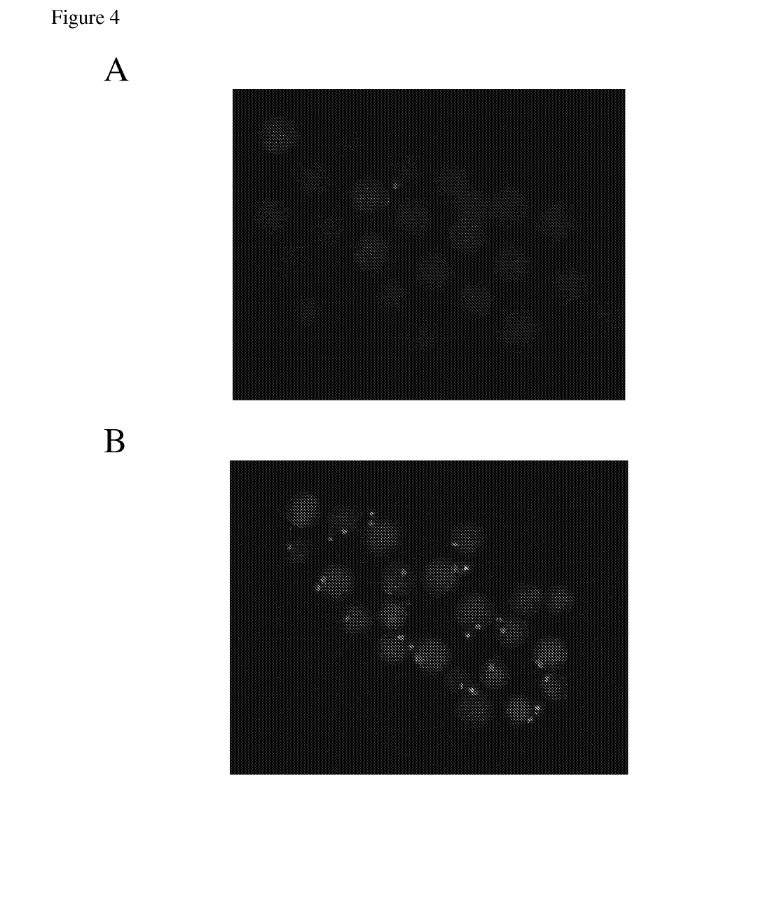

FIG. 4. Hoechst staining and UV illumination of the absence and presence of chromatin. UV light, inverted fluorescent microscopic picture. Bar represents 50 μm. (a) The absence of chromatin in putative cytoplasts (b) The presence of chromatin in putative karyoplasts.

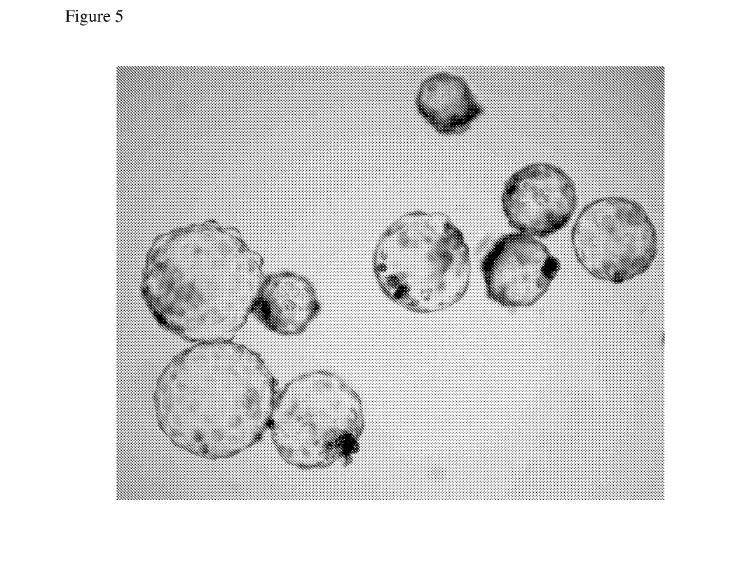

FIG. 5. Stereomicroscopic picture of Day 7 blastocysts produced with chemically assisted handmade enucleation (CAHE). Bar represents 50 μm.

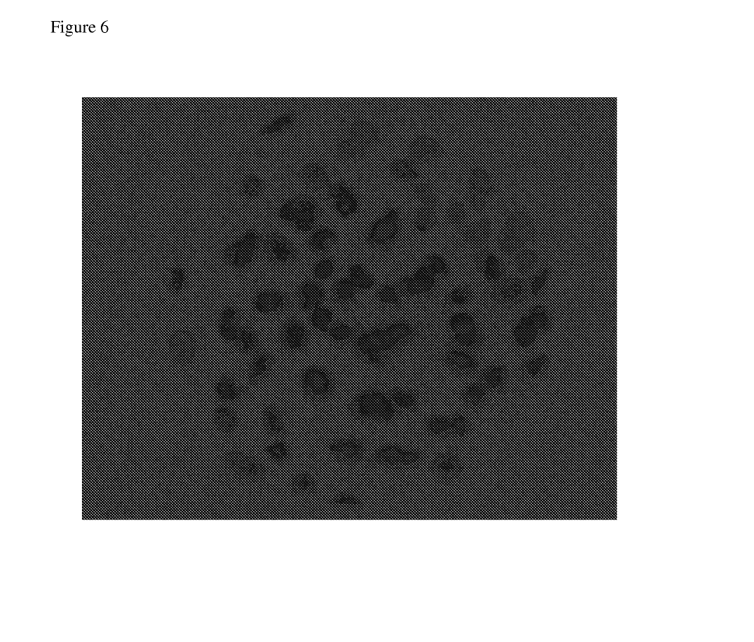

FIG. 6. Hoechst staining and UV illumination of blastocyst developed after chemically assisted handmade enucleation (CAHE). Bar represents 50 μm.

### Mitochondria Related Protein Folding Disorders

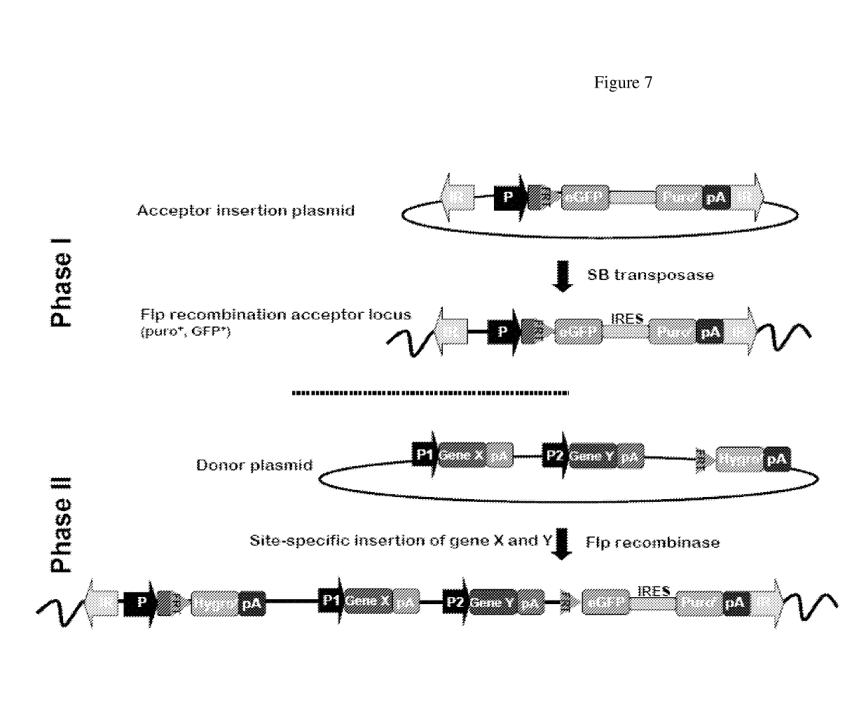

FIG. 7 shows the bi-phased technology of the present invention in which an integrating SB vector, carrying a reporter gene and a selective marker gene, serves as a reporter for continuous gene expression and hence as a target for gene insertion. In a second modification step this vector may serve as a target for insertion of one or more gene expression cassettes in a well-characterized locus.

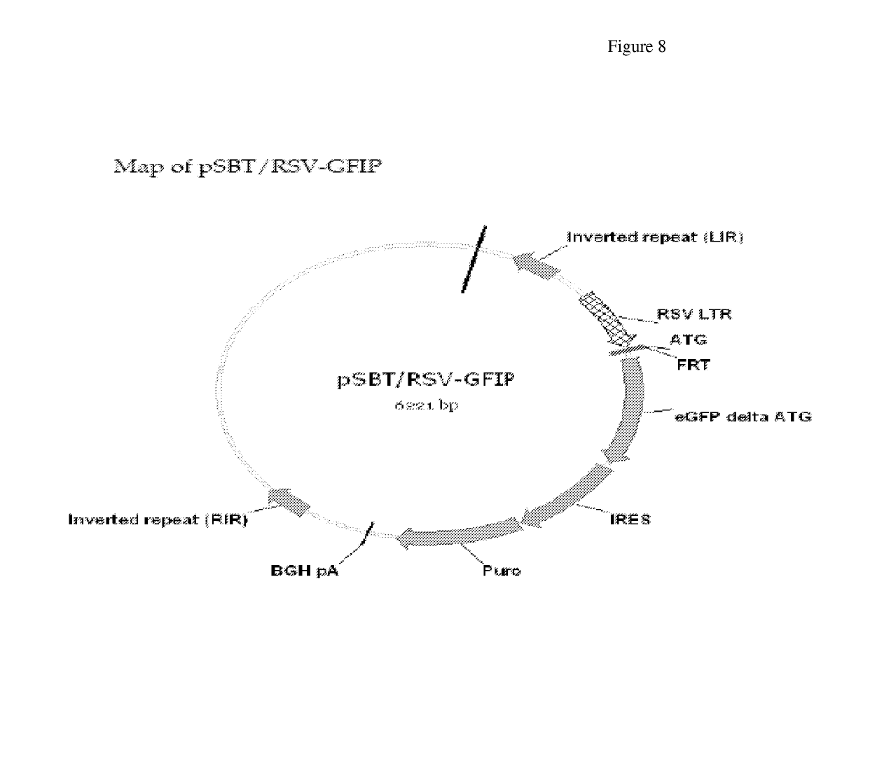

FIG. 8 shows a schematic representation of pSBT/RSV-GFIP.

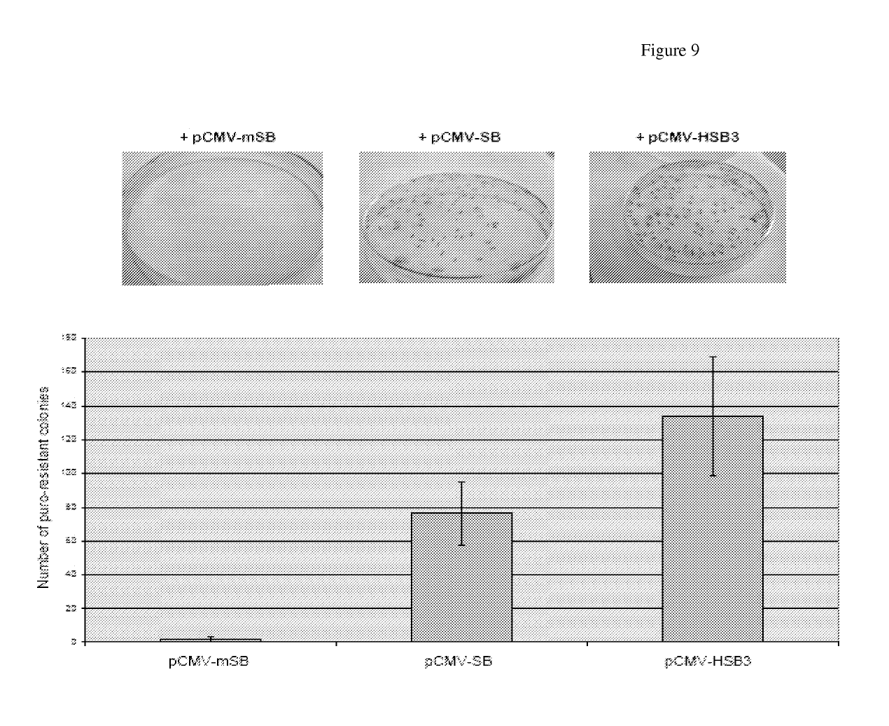

FIG. 9 shows transposition of SB vectors in porcine fibroblasts. A standard transposon encoding a puromycin resistance gene (SBT/PGK-puro) was employed and varying levels of transposition were detected, resulting in about 75 drug-resistant colonies in cultures of fibroblasts co-transfected with pSBT/PGK-puro and pCMV-SB, less than 3 colonies appeared after transfection with pSBT/PGK-puro and pCMV-mSB, the latter which encodes an inactive version of the transposase. Interestingly, a mean of almost 140 colonies was obtained using the hyperactive transposase variant HSB3, indicating that HSB3 also in porcine cells mediates higher levels of transposition compared to the original SB transposase.

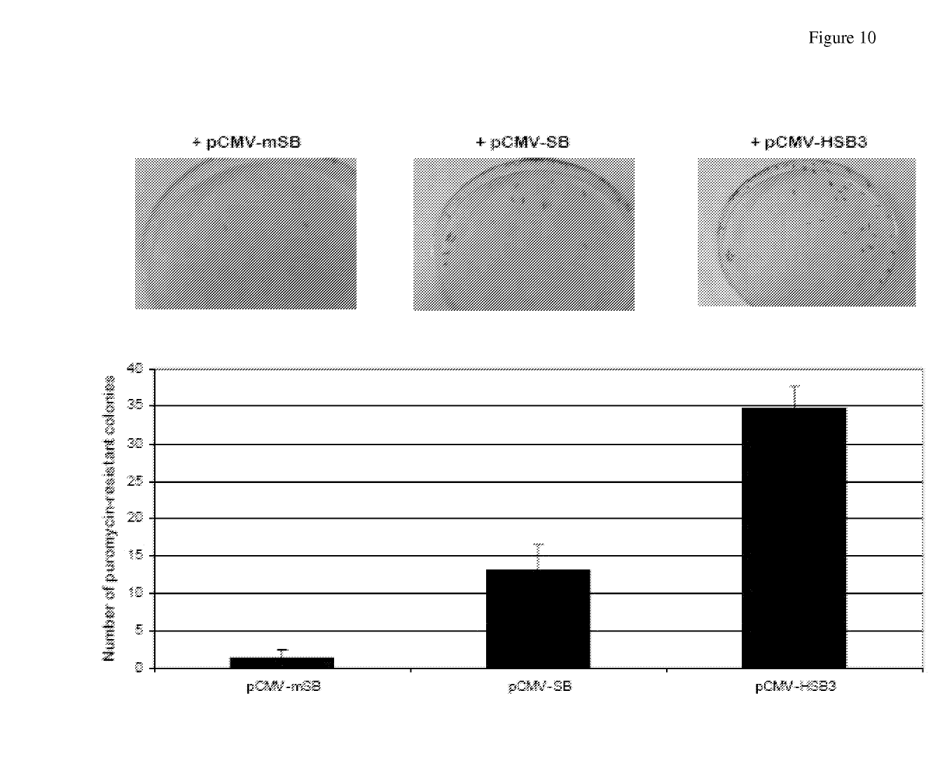

FIG. 10 shows efficient insertion of a FRT-tagged SB vector in pig fibroblasts SB-tagged cell clones containing a Flp recombination target site for site-specific gene insertion were co-transfected the pSBT/loxP.SV40-lopP257 plasmid with pCMV-mSB, pCMV-SB, and pCMV-HSB3, respectively. HSB3 again showed the highest activity, resulting in about 30 drug-resistant colonies after transfection of 3H 10⁴ fibroblasts.

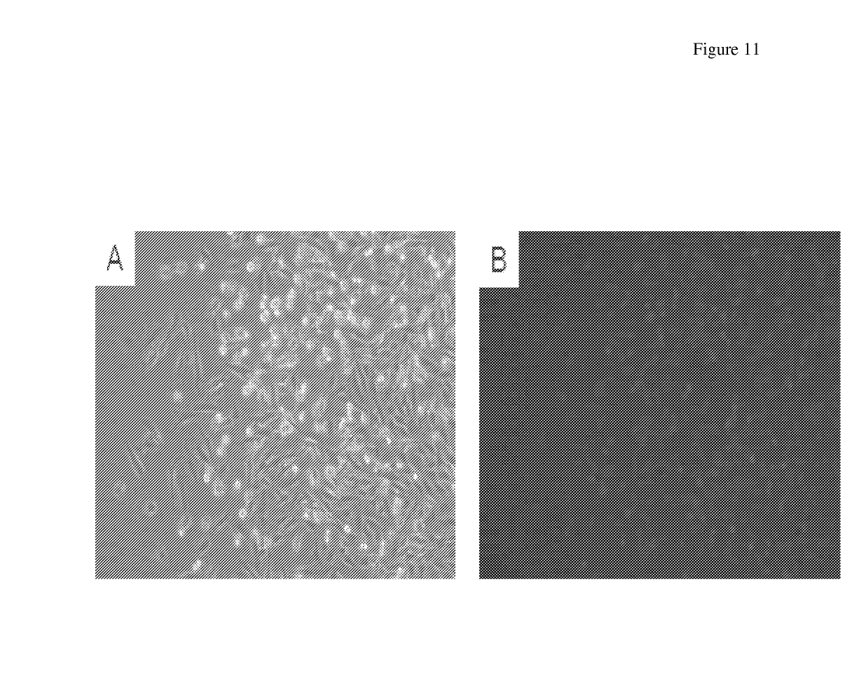

FIG. 11 shows clone analysis by fluorescence microscopy of isolated and expanded puromycin-resistant colonies demonstrates efficient FRTeGFP expression

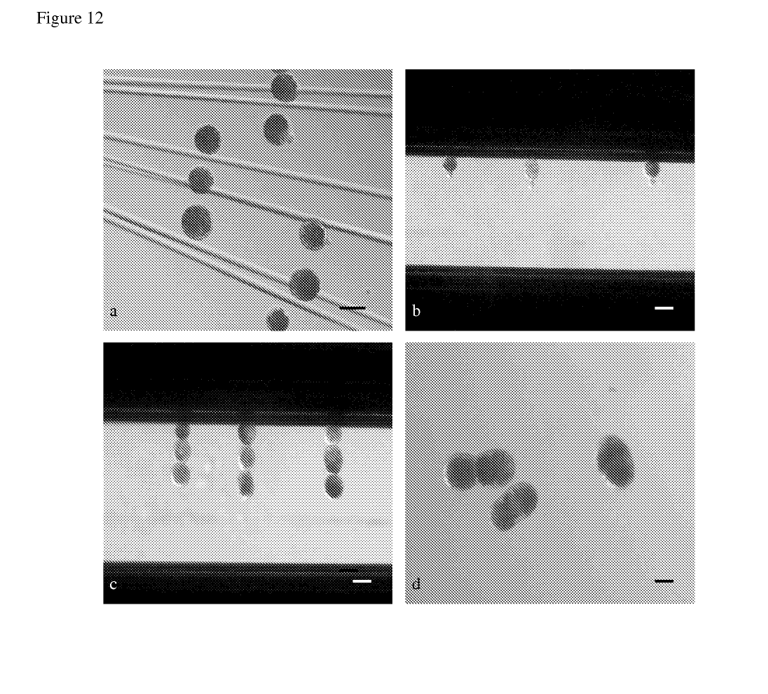

FIG. 12. (a) Oocytes trisection; (b) couplets of fibroblast-oocyte fragment for the first fusion; (c) embryos reconstructed with triplets (note elongation under the AC currency); (d) triplets fusion. Scale bar=50 μm.

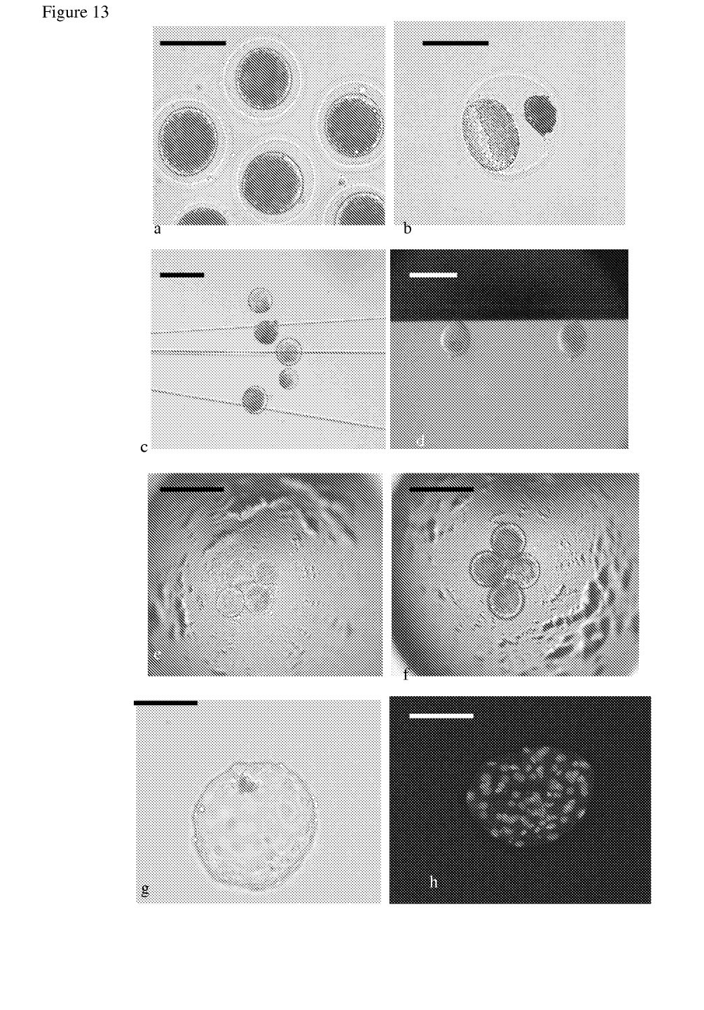

FIG. 13. (a) In vitro matured oocytes after partial zona digestion. (b) Delipated oocytes after centrifugation. (c) Bisection of delipated oocytes. (d) Couplets of fibroblast-oocyte fragment for the first fusion. (e) Four-cell stage reconstructed embryos developed from delipated oocytes. (f) Four-cell stage reconstructed embryos developed from intact oocytes. (g) Re-expanded blastocysts from delipated embryos after warming. (h) Hoechst staining and UV illumination of re-expanded blastocysts from delipated embryos after warming. Bar represents 100 μm.

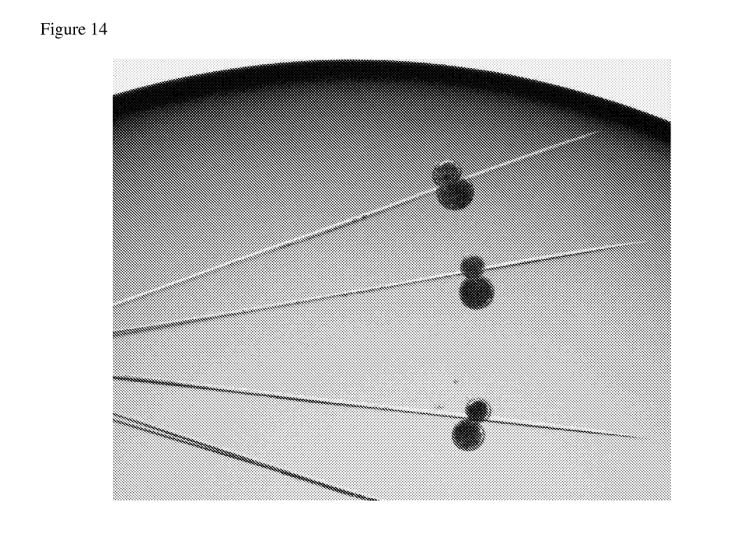

FIG. 14. Bisection at chemically assisted enucleation. Note the extrusion cone or polar body connected to the smaller part (putative karyoplast). Stereomicroscopic picture. Bar represents 50 μm.

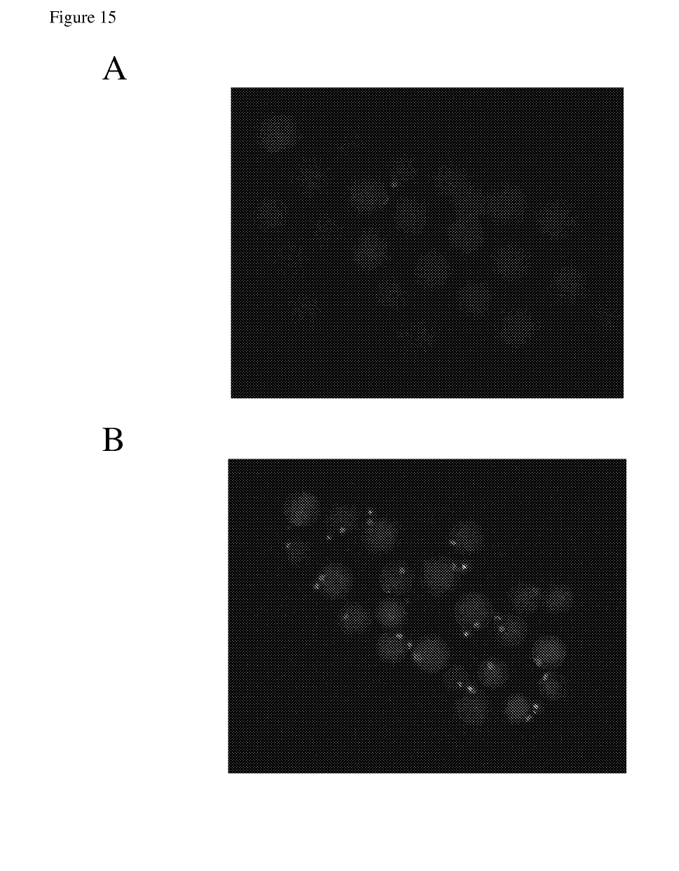

FIG. 15. Hoechst staining and UV illumination of the absence and presence of chromatin. UV light, inverted fluorescent microscopic picture. Bar represents 50 μm. (a) The absence of chromatin in putative cytoplasts (b) The presence of chromatin in putative karyoplasts.

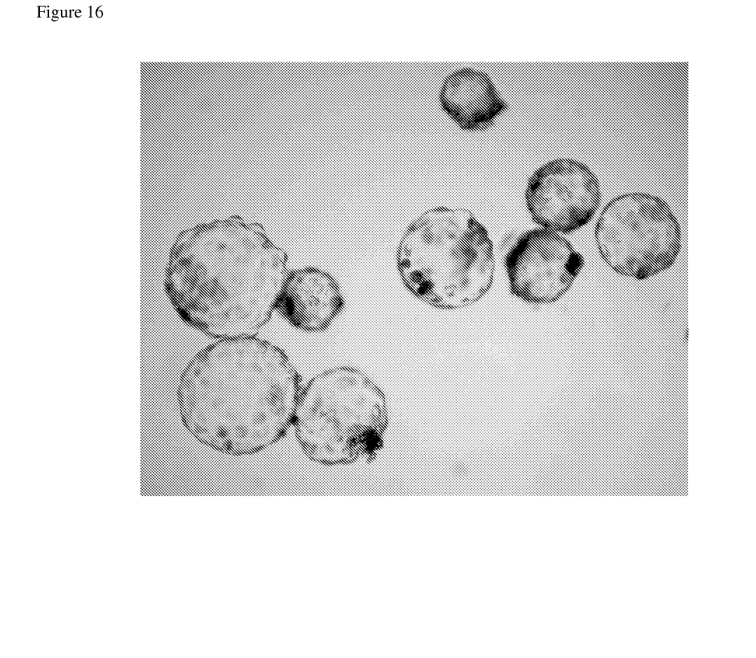

FIG. 16. Stereomicroscopic picture of Day 7 blastocysts produced with chemically assisted handmade enucleation (CAHE). Bar represents 50 μm.

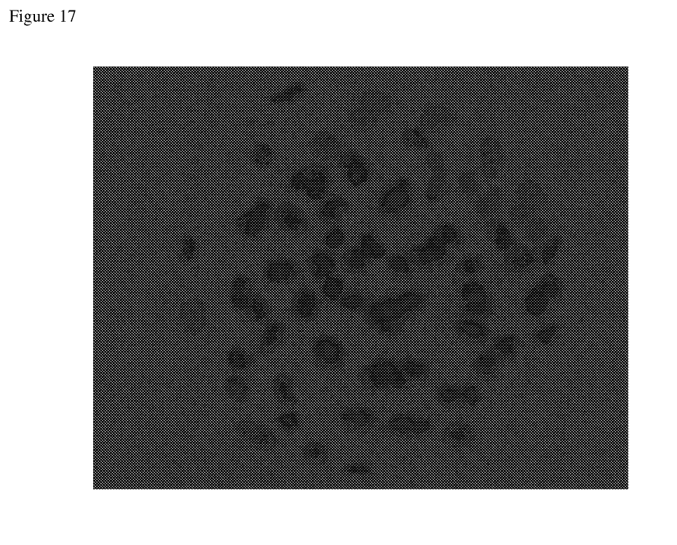

FIG. 17. Hoechst staining and UV illumination of blastocyst developed after chemically assisted handmade enucleation (CAHE). Bar represents 50 μm.

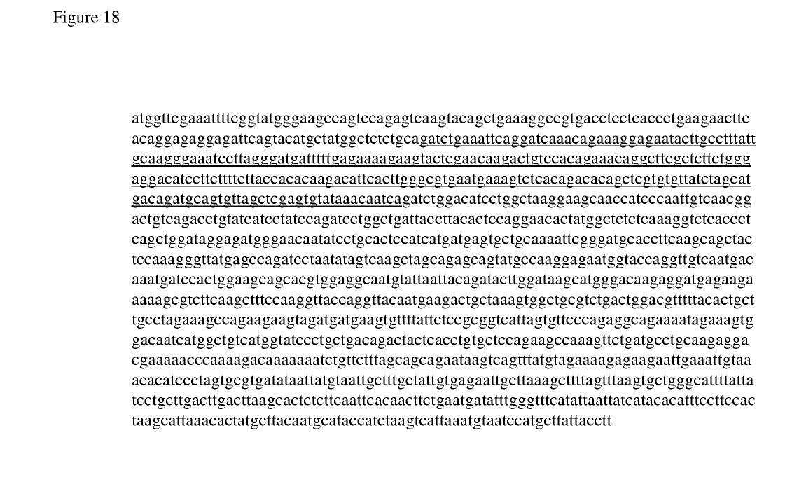

FIG. 18 shows the Rat Otc-Δ cDNA sequence, in which the deleted nucleotides are underlined, cloned into pN1-EGFP (Clonteq) with a CAGGS promoter and as a fusiogene with EGFP (CAGGS-OTCΔ-EGFP and transfected into porcine fetal fibroblasts.

### Epidermolysis Bullosa Simplex

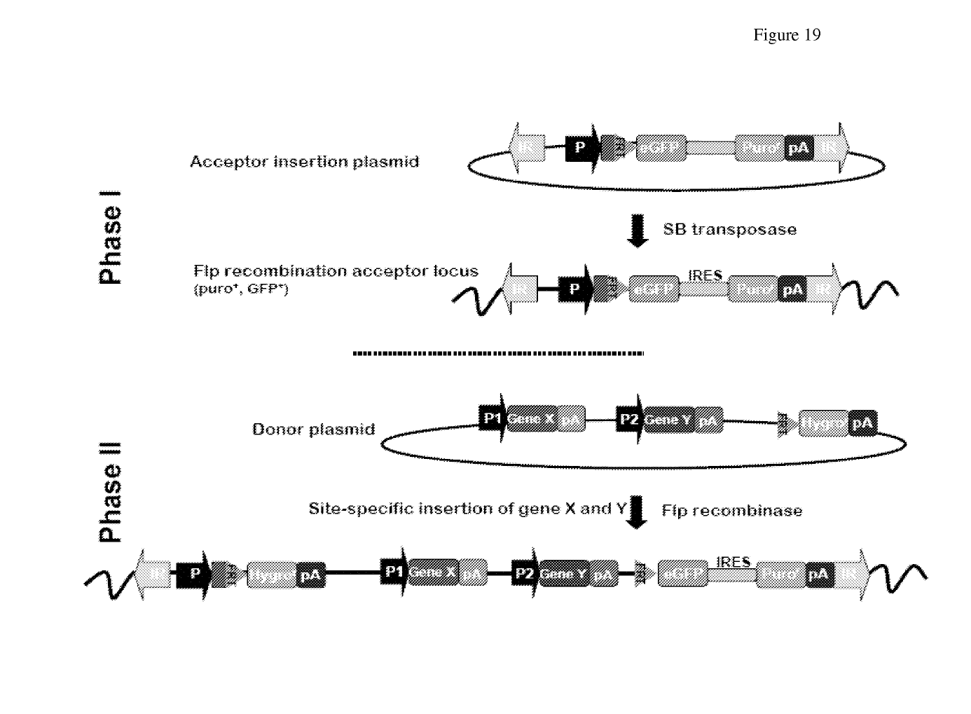

FIG. 19 shows the bi-phased technology of the present invention in which an integrating SB vector, carrying a reporter gene and a selective marker gene, serves as a reporter for continuous gene expression and hence as a target for gene insertion. In a second modification step this vector may serve as a target for insertion of one or more gene expression cassettes in a well-characterized locus.

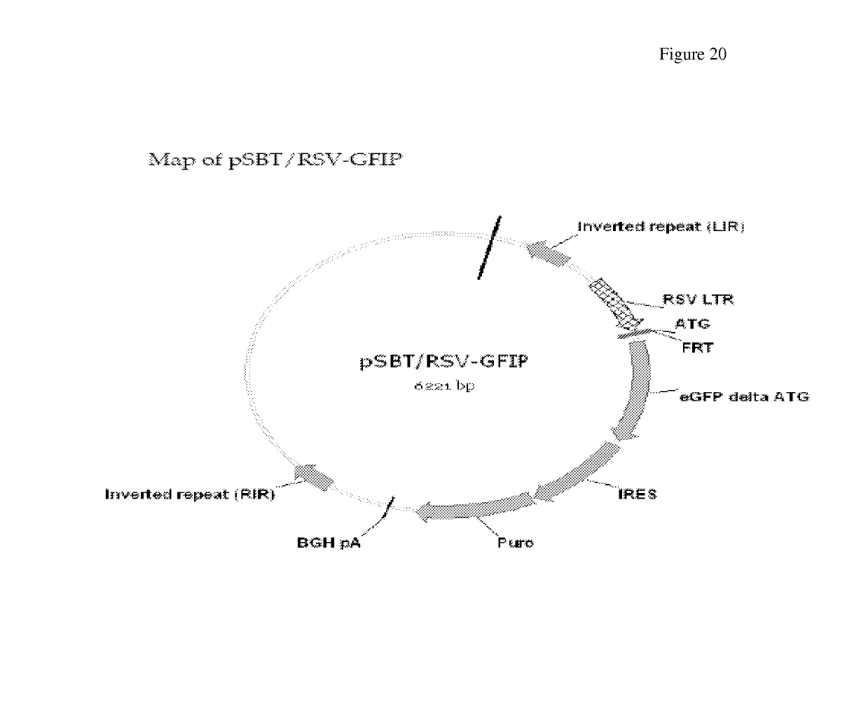

FIG. 20 shows a schematic representation of pSBT/RSV-GFIP.

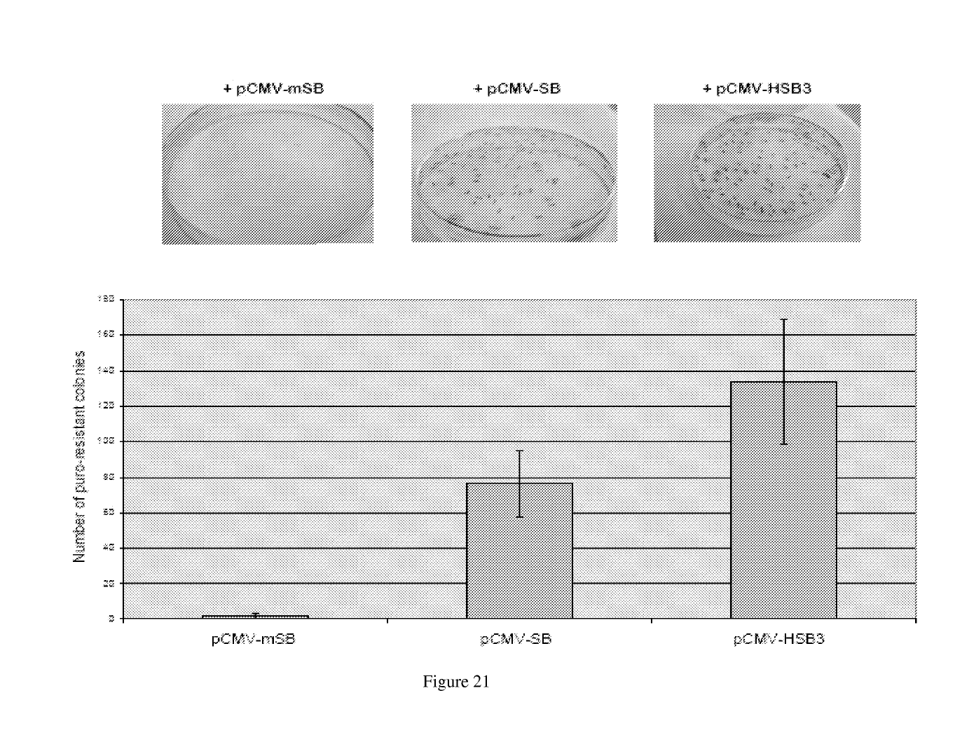

FIG. 21 shows transposition of SB vectors in porcine fibroblasts. A standard transposon encoding a puromycin resistance gene (SBT/PGK-puro) was employed and varying levels of transposition were detected, resulting in about 75 drug-resistant colonies in cultures of fibroblasts co-transfected with pSBT/PGK-puro and pCMV-SB, less than 3 colonies appeared after transfection with pSBT/PGK-puro and pCMV-mSB, the latter which encodes an inactive version of the transposase. Interestingly, a mean of almost 140 colonies was obtained using the hyperactive transposase variant HSB3, indicating that HSB3 also in porcine cells mediates higher levels of transposition compared to the original SB transposase.

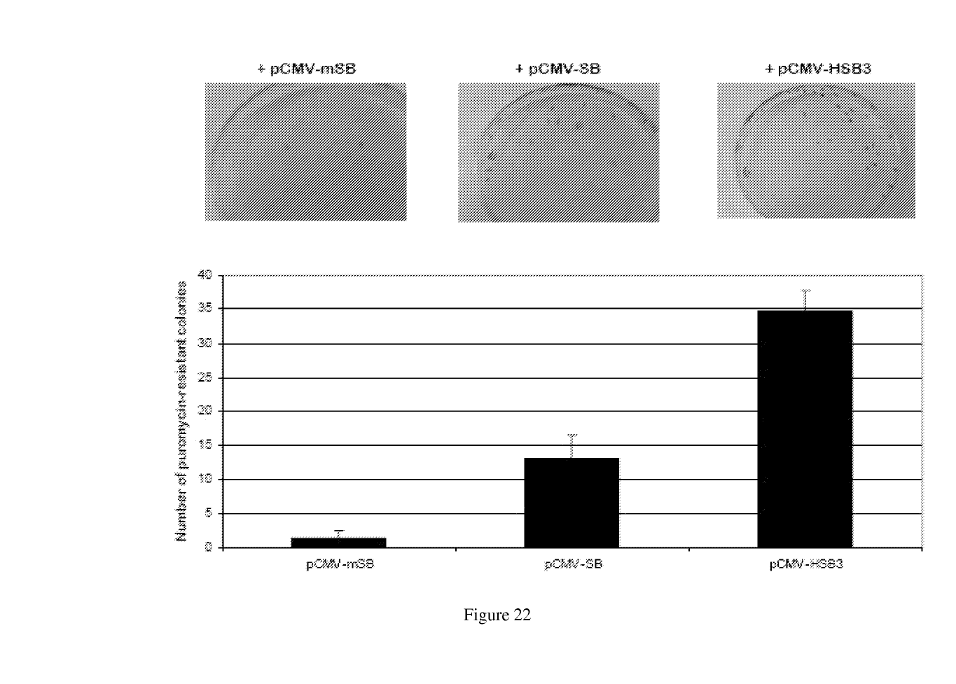

FIG. 22 shows efficient insertion of a FRT-tagged SB vector in pig fibroblasts SB-tagged cell clones containing a Flp recombination target site for site-specific gene insertion were co-transfected the pSBT/loxP.SV40-lopP257 plasmid with pCMV-mSB, pCMV-SB, and pCMV-HSB3, respectively. HSB3 again showed the highest activity, resulting in about 30 drug-resistant colonies after transfection of 3H 10⁴ fibroblasts.

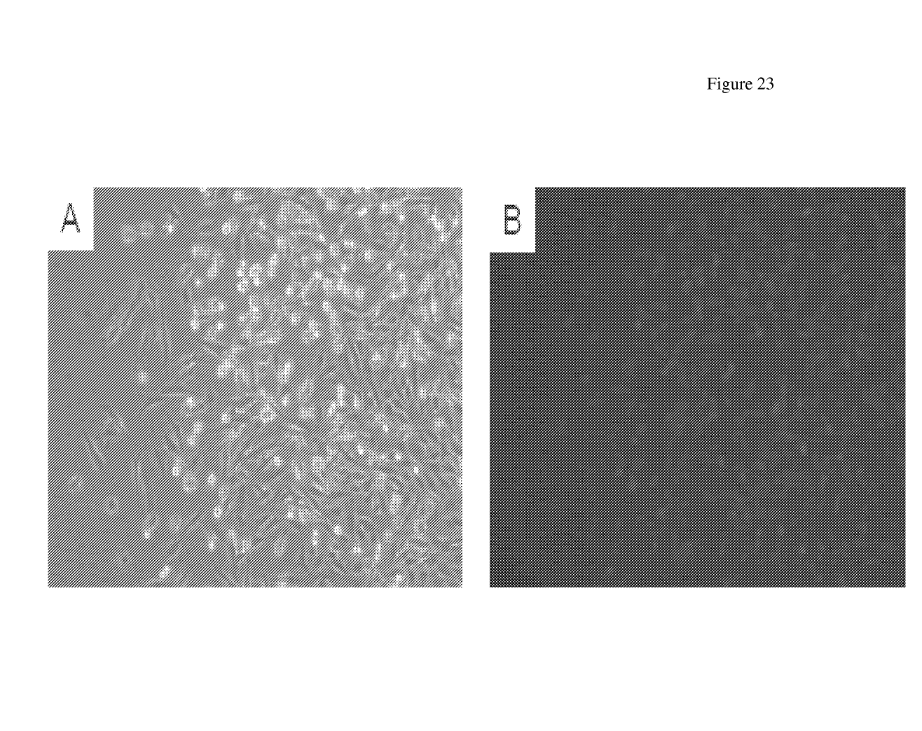

FIG. 23 shows clone analysis by fluorescence microscopy of isolated and expanded puromycin-resistant colonies demonstrates efficient FRTeGFP expression

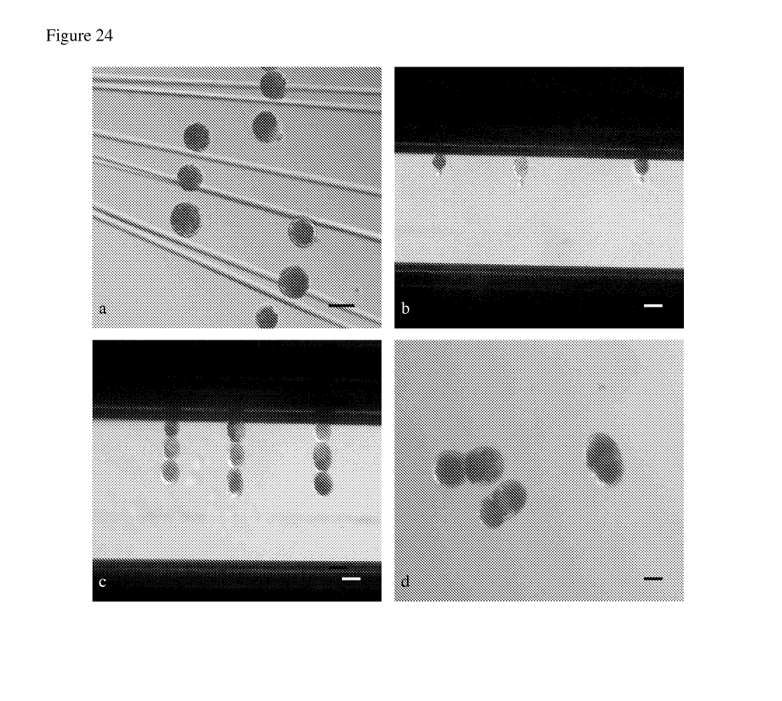

FIG. 24. (a) Oocytes trisection; (b) couplets of fibroblast-oocyte fragment for the first fusion; (c) embryos reconstructed with triplets (note elongation under the AC currency); (d) triplets fusion. Scale bar=50 μm.

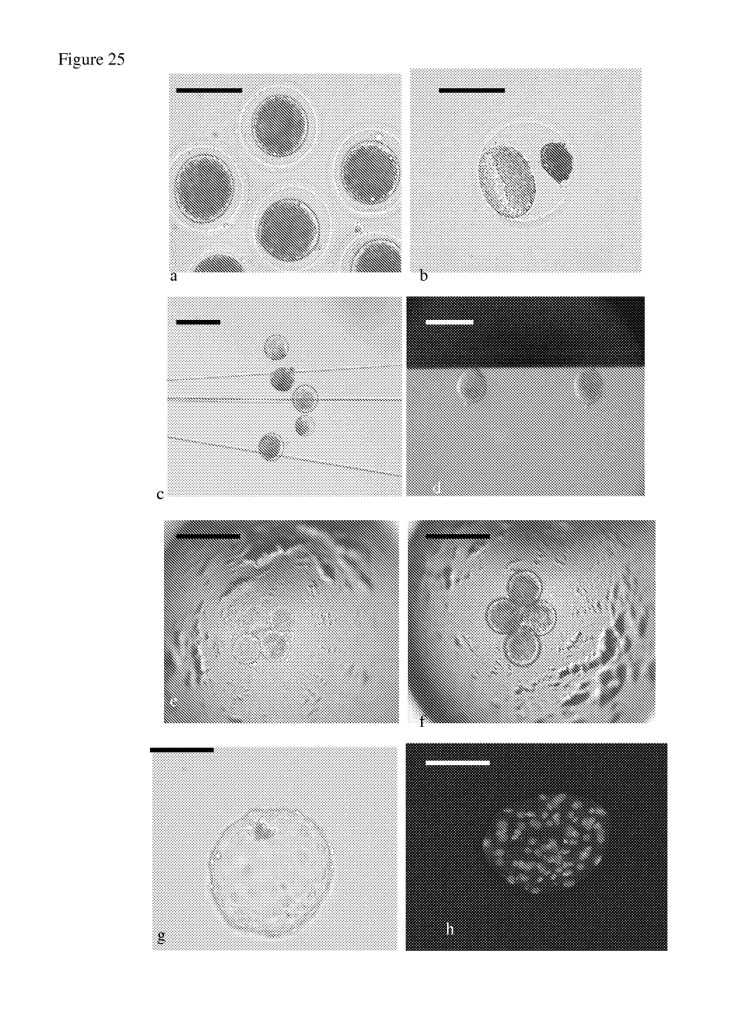

FIG. 25. (a) In vitro matured oocytes after partial zona digestion. (b) Delipated oocytes after centrifugation. (c) Bisection of delipated oocytes. (d) Couplets of fibroblast-oocyte fragment for the first fusion. (e) Four-cell stage reconstructed embryos developed from delipated oocytes. (f) Four-cell stage reconstructed embryos developed from intact oocytes. (g) Re-expanded blastocysts from delipated embryos after warming. (h) Hoechst staining and UV illumination of re-expanded blastocysts from delipated embryos after warming. Bar represents 100 μm.

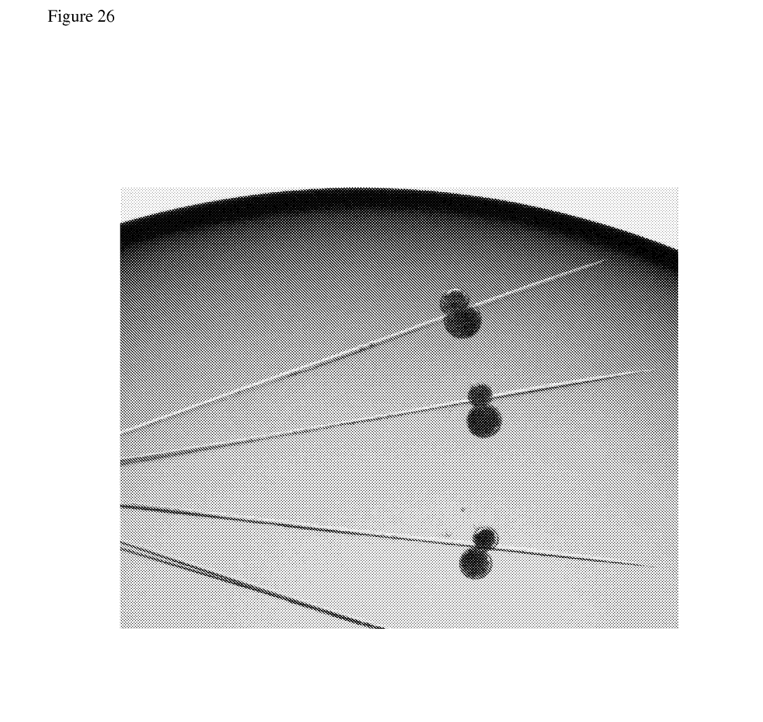

FIG. 26. Bisection at chemically assisted enucleation. Note the extrusion cone or polar body connected to the smaller part (putative karyoplast). Stereomicroscopic picture. Bar represents 50 μm.

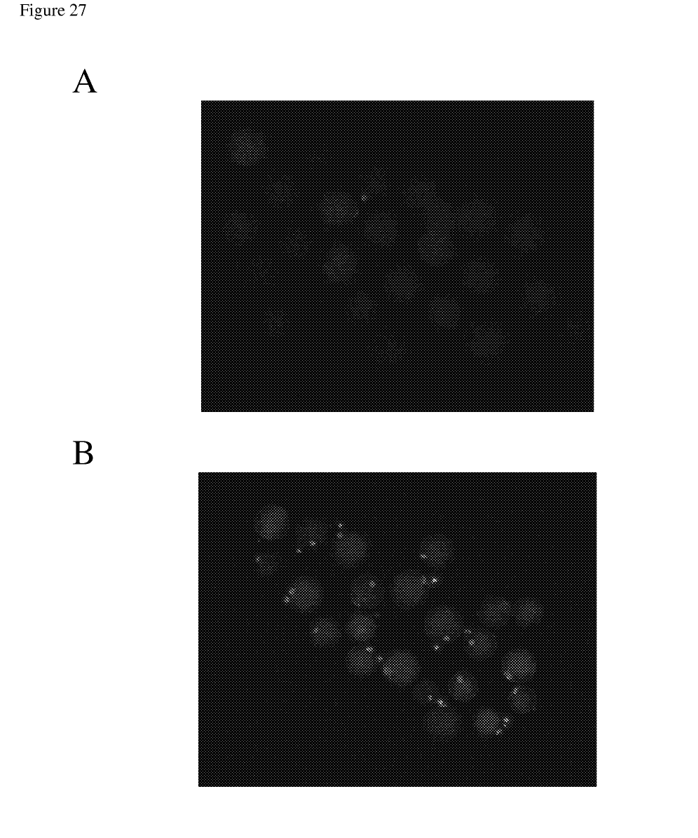

FIG. 27. Hoechst staining and UV illumination of the absence and presence of chromatin. UV light, inverted fluorescent microscopic picture. Bar represents 50 μm. (a) The absence of chromatin in putative cytoplasts (b) The presence of chromatin in putative karyoplasts.

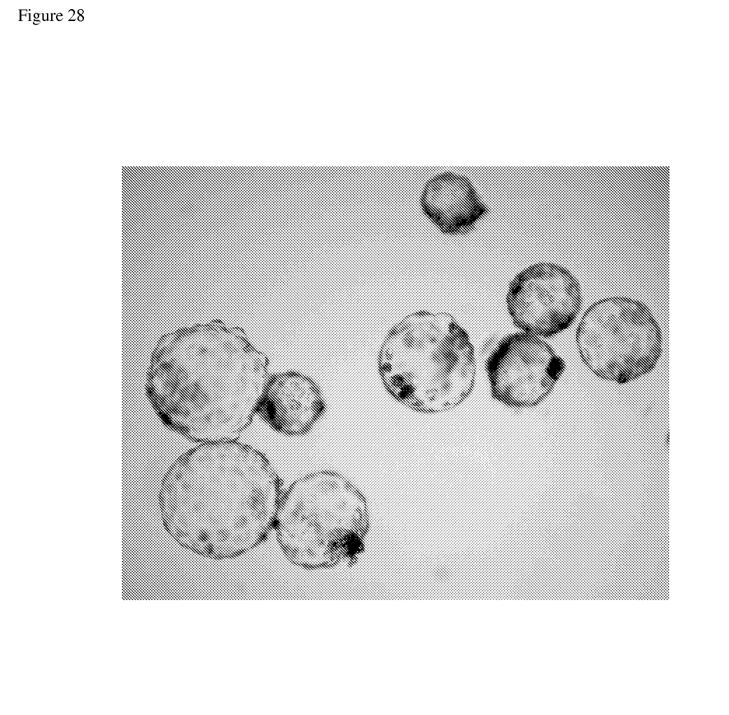

FIG. 28. Stereomicroscopic picture of Day 7 blastocysts produced with chemically assisted handmade enucleation (CAHE). Bar represents 50 μm.

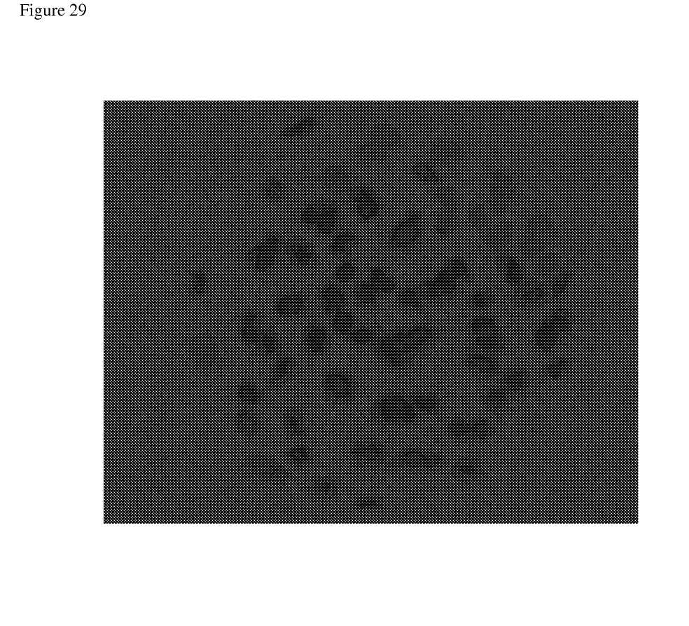

FIG. 29. Hoechst staining and UV illumination of blastocyst developed after chemically assisted handmade enucleation (CAHE). Bar represents 50 μm.

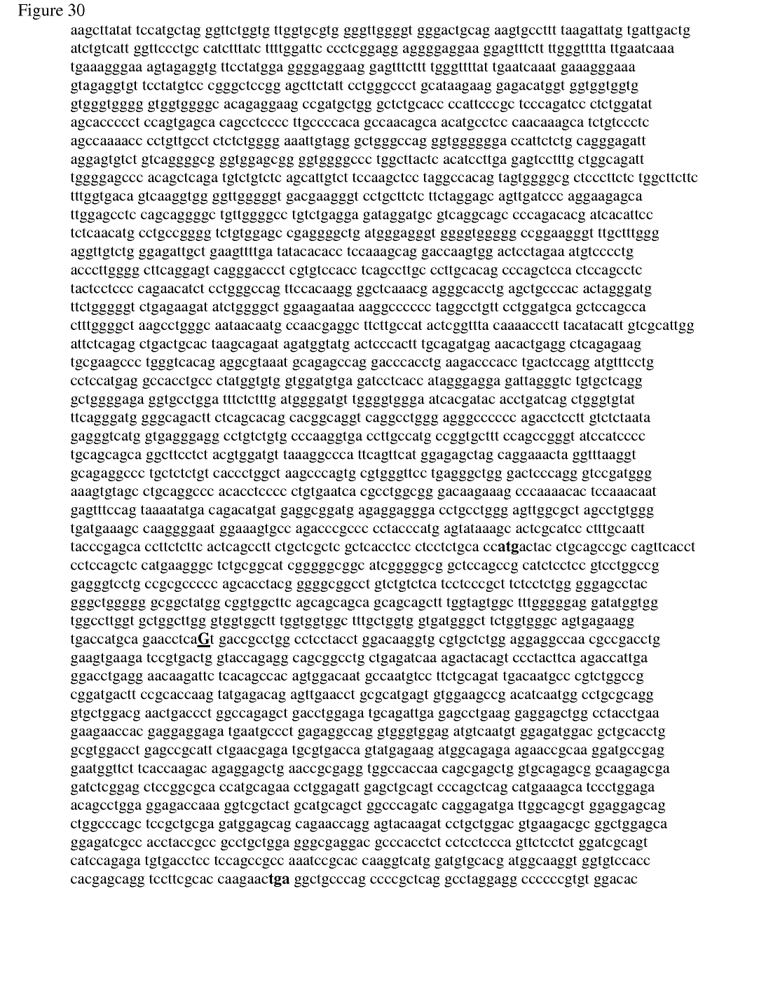

FIG. 30 shows the sequence of the transgene integrated in porcine fetal fibroblasts causing Epidermolysis Bullosa Simplex: human keratin 14 promoter and keratin 14 cDNA including start and stop codons (in bold) and the disease-causing mutation (in bold and underlined)

### DETAILED DESCRIPTION OF THE INVENTION

The present invention pertains to a genetically modified pig model for studying breast cancer, mitochondria related protein folding disorders and/or epidermolysis bullosa simplex, wherein the pig model expresses at least one phenotype associated with breast cancer, mitochondria related protein folding disorders and/or epidermolysis bullosa simplex.

It will be appreciated that the invention does not comprise processes for modifying the genetic identity of pigs which are likely to cause them suffering without any substantial medical benefit to man or animal, or animals resulting from such processes.

The present invention also relates to modified pig embryos, blastocysts, donor cells and/or fetuses obtainable by the methods described herein.

The methods for producing the pig model for studying breast cancer, mitochondria related protein folding disorders and/or epidermolysis bullosa simplex described herein do not encompass a surgical step performed on the pig.

The term “genetic determinant” is used herein to refer to a single-stranded or double-stranded “polynucleotide molecule” or “nucleic acid” comprising a structural gene of interest. The “genetic determinant” encodes a protein not ordinarily made in appreciable amounts in the target cells. Thus, “genetic determinants” include nucleic acids which are not ordinarily found in the genome of the target cell. “Genetic determinants” also include nucleic acids which are ordinarily found within the genome of the target cell, but is in a form which allows for the expression of proteins which are not ordinarily expressed in the target cells in appreciable amounts. Alternatively, “genetic determinants” may encode a variant or mutant form of a naturally-occurring protein.

The terms “polynucleotide” and “nucleic acid” are used interchangeably, and, when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

### Pigs

The present invention relates to a modified pig as a model for studying breast cancer, mitochondria related protein folding disorders and/or epidermolysis bullosa simplex, wherein the pig model expresses at least one phenotype associated with breast cancer, mitochondria related protein folding disorders and/or epidermolysis bullosa simplex. The pig of the present invention may be any pig.

The pig is evolutionary close to humans as compared to for example rodentia. Furthermore, the pig has been widely used in bio-medical research because of the similarities between human and porcine physiology (Douglas, 1972; Book & Bustad, 1974).

In one embodiment the pig of the present invention is a wild pig. In another embodiment the pig is the domestic pig, Sus scrofa, such as S. domesticus. In yet another embodiment the invention relates to mini pigs, as well as to inbred pigs. The pig can be selected e.g. from the group consisting of Landrace, Yorkshire, Hampshire, Duroc, Chinese Meishan, Berkshire and Piêtrain, such as the group consisting of Landrace, Yorkshire, Hampshire and Duroc, for example the group consisting of Landrace, Duroc and Chinese Meishan, such as the group consisting of Berkshire, Pietrain, Landrace and Chinese Meishan, for example the group consisting of Landrace and Chinese Meishan. In one embodiment, the pig is not a mini-pig.

In another embodiment the pig of the present invention is an inbred pig.

In another embodiment of the present invention the pig is a mini-pig and the mini-pig is preferably selected from the group consisting of Goettingen, Yucatan, Bama Xiang Zhu, Wuzhishan and Xi Shuang Banna. Thus, the present invention relates to any of Goettingen, Yucatan, Bama Xiang Zhu, Wuzhishan and Xi Shuang Banna separately or in any combination.

Due to its size and weight of about 200 kg the domestic pig is not easily handled in a laboratory setting. A preferred alternative to the domestic pig is the Goettingen (Göttingen) mini-pig that weighs about 30 kg. Therefore, a preferred embodiment the pig of the present invention is the Goettingen mini pig.

### Genetically Modified

The modifications are introduced in the somatic cell prior to cell nuclear transfer. However, the genetic modification may in another embodiment be introduced during the cell nuclear transfer process, for example by addition of transgenes at different steps of the hand made cloning (HMC) procedure that will then find their way to the genome of the embryo.

The genetic modifications comprise random integration of a disease causing gene, mutated gene, into the genome of the somatic cell. It could also be random integration of a normal non-mutated gene that will cause a disease when expressed in a specific tissue or at a specific expression level.

However, the invention also pertains to modified pigs, embryos, donor cells, blastocysts and/or fetuses obtained by transfer of mRNA and/or protein of the genes disclosed herein. Thus, the modification of the pig is in one embodiment does not lead to integration of a transgene into the genome of the pig, embryo, blastocyst and/or fetus.

The introduced gene or transgene, transcriptional and/or translational product or part thereof may originate from any species, including bacteria, pig, human, mouse, rat, yeast, invertebrates, or plants. Regulatory sequences of the transgene may drive ubiquitous or inducible or tissue- and/or time-specific expression and may also originate from any species including pig, human, mouse, rat, yeast, invertebrates, or plants.

Importantly, the genetic modification in the somatic cell may be targeted to a specific region in the porcine genome by homologous recombination of a targeting construct or by gene editing procedures. This could be inactivation (e.g. knock-out) of specific genes that will cause a disease or phenotype, or it could be integration (knock-in) of specific mutations to specific genes that will then cause disease. Also, disease causing transgenes can be integrated into specific regulatory regions of the porcine genome by homologous recombination methods.

Homologous recombination occurs between two homologous DNA molecules. It is also called DNA crossover. By homologous recombination, one DNA segment can replace another DNA segment with a similar sequence. The process involve breakage and reunion between the homologous regions of DNA, which is mediated by specialized enzymes. The technique allows replacing one allele with an engineered construct without affecting any other locus in the genome. Using homologous recombination it is possible to direct the insertion of a transgene to a specific known locus of the host cells genome. Knowing the DNA sequence of the target locus, it is possible to replace any gene with a genetically modified DNA construct, thereby either replacing or deleting the target sequence. The technique comprises discovering and isolating the normal gene and then determining its function by replacing it in vivo with a defective copy. This procedure is known as ‘gene knock-out’, which allows for specific gene targeting by taking advantage of homologous recombination. Cloned copies of the target gene are altered to make them nonfunctional and are then introduced into ES cells where they recombine with the homologous gene in the cell's genome, replacing the normal gene with a nonfunctional copy.

Homologous recombination can similarly be exploited to generate fusion genes or insertion of point mutations in a ‘knock-in’ strategy, in which a targeting vector, comprising a relevant exon of the target locus fused with the cDNA sequence of chromosomal translocation-fusion partner, is transfected into embryonic stem cells, whereby the recombinant sequence is fused to an endogenous gene to generate fusion a gene.

Another applicable technique to exploits the phenomenon called RNA interference (RNAi), in which 21 nucleotide small interfering RNAs (siRNA) can elicit an effective degradation of specific mRNAs. RNA interference constitutes a new level of gene regulation in eukaryotic cells. It is based on the fact that presence of double stranded RNA in a cell eliminates the expression of a gene of the same sequence, whereas expression of other unrelated genes is left undisturbed. The siRNA stimulates the cellular machinery to cut up other single-stranded RNA having the same sequence as the siRNA.

The genetic modifications introduced into the porcine genome prior or during the HMC procedure could also be epigenetic modifications (e.g. methylation of DNA or methylation or acetylation/deacetylation of histones) by incubating somatic cells, oocytes or reconstructed HMC embryos with chemical components such as Tricostatin or compounds with similar effect.

The present invention relates to a modified pig, comprising a genetic determinant in the form of modified exon 3 or part thereof of the BRCA1 gene and/or porcine BRCA1 comprising a nucleotide substitution from T to G resulting in amino acid substitution from Cys to Gly at codon 61 of exon 3 and/or exon 11 or part thereof of the BRCA1 gene and/or porcine BRCA1 gene comprising a deletion of at least one allele of exon 11 or part thereof of the BRCA1 gene and/or exon 11 or part thereof of the BRCA2 gene, and/or porcine BRCA2 gene comprising a deletion of at least one allele of exon 11 or part thereof of the BRCA2 gene and/or rat Ornithine TransCarbamylase (OTC) gene or part thereof, and/or human Ornithine TransCarbamylase gene or part thereof, and/or porcine Ornithine TransCarbamylase gene or part thereof, and/or rat Ornithine TransCarbamylase cDNA or part thereof, and/or porcine Ornithine TransCarbamylase cDNA or part thereof, and/or human Ornithine TransCarbamylase cDNA or part thereof, and/or porcine keratin 14 gene or part thereof, and/or human keratin 14 gene or part thereof, and/or porcine keratin 14 cDNA or part thereof, and/or human keratin 14 cDNA or part thereof, and/or a transcriptional and/or translational product thereof, separately or in combination as described in detail herein. The present invention also relates to porcine embryos, blastocysts and/or fetuses derived from a modified pig expressing at least one phenotype associated with Alzheimer's disease.

### Breast Cancer

In one embodiment of the present invention the transgenic pig, embryo, blastocyst, donor cell and/or fetus is transgenic for at least one codon of the endogenous BRCA1 gene or part thereof, namely at codon 61 BRCA1. The porcine BRCA1 exon 3 nucleotide substitution from T to G results in amino acid substitution from Cys to Gly (codon 61). The nucleotide fragment with the sequence (SEQ ID NO: 1) tttngtatgctgaaacttctcaaccagaagaaagggccttcacagT>Ggtcctttgtgtaagaatgatataaccaaaagg is introduced into the endogenous porcine BRCA1 gene by homologous recombination in a somatic porcine cell, for example a porcine fibroblast cell.

In another embodiment of the present invention the transgenic pig, embryo, blastocyst, donor cell and/or fetus is transgenic for one allele of the porcine BRCA2 gene, wherein all or part of exon 11 of the porcine BRCA2 gene is deleted by homologous recombination of a construct containing a selection gene inside exon 11 sequence of BRCA2 gene into the endogenous BRCA2 gene. The region of the porcine BRCA2 exon 11 to be deleted is the sequence (SEQ ID NO: 2)

```
      1 ggtccaggat gtttctcttc aagcaaatgt aatgattctg atgtttcaat atttaaggta
    61 gaaaattata gcagtgataa aagtttaagt gagaaataca ataaatgcca actgatacta
  121 aaaaataaca ttgaaaggac tgctgacatt tttgttgaag aaaatactga cggttacaag
  181 agaaatactg aaaataaaga caacaaatgt actggtcttg ctagtaactt aggaggaagc
  241 tggatggaca gtgcttcaag taaaactgat acagtttata tgcacgaaga tgaaactggt
  301 ttgccattta ttgatcacaa catacateta aaattaccta accactttat gaagaaggga
  361 aatactcaaa ttaaagaagg tttgtcagat ttgacttgtt tggaagttat gagagccgaa
  421 gaaacatttc atattaatac atcaaataaa cagtcaactg ttaataagag gagccaaaaa
  481 ataaaagatt ttgatgtttt tgatttgtcc tttcagagtg caagtgggaa aaacatcaga
  541 gtctctaaag agtcattaaa taaagctgta aatttctttg acgaaaaatg cacagaagaa
  601 gaattgaata acttttcaga ttcctcaaat tctgaaatac ttcctggcat aaatatcaac
  661 aaaataaaca tttcaagcca taaggaaaca gattcggaca aaaacaaact attgaaagaa
  721 agtgacccag ttggtattga aaatcaatta ctgactctcc agcaaagatc agaatgtgaa
  781 atcaaaaaga tcgaagaacc taccatgctg ggttttcata cagctagtgg gaaaaaagta
  841 aaaattgcga aggaatcgtt ggacaaagtg aaaaatcttt ttgatgaaac aaagcaagat
  901 agtagtgaaa ccactaattc tagccatcaa ggggtaaaaa cacagaagga cagagaggta
  961 tgtaaagaag agcttgaatt aacattcgag acagttgaaa taactgcctc aaagcatgaa
1021 gaaatacgga attttttaga ggagaaaaaa cttgtttcta aggagatcac catgccaccc
1081 aggctcttac gtcatcattt acacagacaa actgaaaatc tcagcatgtc aaacagtatc
1141 cccctaaaag gtaaagtaca tgaaaatatg gaagaagaaa catcttgtca cacagatcag
1201 tccacttgtt cagccattga aaattcagca ttaacatttt acacaggaca tggcagaaaa
1261 atttctgtga atcaggcttc cgtatttgaa gccaaaaagt ggcttagaga aggagaattg
1321 gacgatcaac cagaaaacgt agattctgcc aaggtcatat gtttaaagga atatgctagg
1381 gattatgtag gaaatccttt gtgtgggagt agttcaaaca gtatcataac tgaaaatgac
1441 aaaaatctcc ctgaaaaaca aaattcaact tatttaagta acagtgtgtc taacaactat
1501 tcataccatt ctgatttttg tcattccaat gaggtgctca gcaaatcaga atctctctca
1561 gaaaataaaa ttggtaattc tgatactgag ccagcagtga agaatgtcaa agacagaaaa
1621 gacacttgtt tttctgaaga gatatccacc gtaagagaag caaacacaca cccacaagct
1681 gtagatgaag acagctgggt tcggaagctt gtgattaact ctacaccatg caaaaataaa
1741 aatacacctg gtgaagtgtc caatctaatt caaataattt tgagatagag ccacctgcat
1801 tcagtacaag tgggaacata gcctttgttt cacatgaaac agacgtgaga gagaggtttg
1861 cagacaacaa caggaaggcg attaagcaaa acactgagag tatgtcaggc tcttgccaaa
1921 tgaaaattat gactggcgct cataaggcat tgggtgattc agaggatgtt attttcccta
1981 actctccaga tagtgaagaa catattacac gttcacagga ggtttttcct gaaattcaaa
2041 gtgaacaaat tttacaacat gacccaagtg tatccggatt ggagaaagtt tctgaaatgc
2101 caccttgtca tattaactta aaaacttttg atatacataa gtttgatatg aaaagacatc
2161 ccatgtcagt ctcttctatg aatgattgtg gggtttttag cacagcaagt ggaaaatctg
2221 tacaagtatc agatactgca ttacaaaaag cgagacaagt attttctaag acagaagatg
2281 tggctaagcc attcttttcc agagcagtta aaagtgatga agaacattca gacaagtaca
2341 caagagaaga aaatgctatg atgcatcccc ccccaaattt cctgtcatct gctttctccg
2401 gatttagtac agcaagtgga aaacaggttc cagtttctga gagtgcctta tgcaaagtga
2461 agggaatgtt tgaggaattt gatttaatgg gaactgaatg tagacttcag cattcaccta
2521 catctagaca agatgtgtca aagatacttc ctctctccga gattgatgag agaaccccag
2581 aacactctgt aagttcccaa acagagaaag cctacaatga acaatttaaa ttaccagata
2641 gctgtaacac tgaaagcagt tcttcagaaa ataatcactc tgttaaagtt tctcccgatc
2701 tctctcggtt taagcaagac aaacagttgg tatcaggagc aaaagtatca cttgttgaga
2761 acattcatcc atcgggaaaa gaa
```

However, in another embodiment of the present invention the region of the porcine BRCA2 exon 11 to be deleted corresponds to nucleotides 1 to 500 of SEQ ID NO:2), 501 to 1000, 1001 to 1500, 1501 to 2000, or 2001 to 2761 of SEQ ID NO:2. Alternatively, the region of the porcine BRCA2 exon 11 to be deleted corresponds to nucleotides 1 to 100, 101 to 200, 201 to 300, 301 to 400, 401 to 500 of SEQ ID NO.:2, 501 to 600, 601 to 700, 701 to 800, 801 to 900, 901 to 1000 of SEQ ID NO.:2, 1001 to 1200, 1201 to 1300, 1301 to 1400, 1401 to 1500, 1501 to 1600, 1601 to 1700, 1701 to 1800, 1801 to 1900, 1901 to 2000, 2001 to 2200, 2201 to 2300, 2301 to 2400, 2401 to 2500, 2501 to 2600, 2601 to 2761 of SEQ ID NO.:2.

In another embodiment of the present invention the transgenic pig, embryo, blastocyst, donor cell and/or fetus is transgenic for a deletion of exon 11 of the endogenous porcine BRCA1 gene. The sequence of the porcine exon 11 which is to be deleted corresponds to SEQ ID NO: 3:

```
    1 agcatgagac cagcagttta ttactcacta aagacagaat gaatgtagaa aaggctgaat
  61 tttgtaataa aagcaagcag cctgtcttag caaagagcca acagagcaga tgggctgaaa
121 gtaagggcac atgtaatgat aggcagactc ctaacacaga gaaaaaggta gttctgaata
181 ctgatctcct gtatgggaga aacgaactga ataagcagaa acctgcgtgc tctgacagtc
241 ctagagattc ccaagatgtt ccttggataa cattgaatag tagcatacag aaagttaatg
301 agtggttttc tagaagcgat gaaatgttaa cttctgacga ctcacaggac aggaggtctg
361 aatcaaatac tggggtagct ggtgcagcag aggttccaaa tgaagcagat ggacatttgg
421 gttcttcaga gaaaatagac ttaatggcca gtgaccctca tggtgcttta atacgtgaac
481 gtgaaagagg gcactccaaa ccagcagaga gtaatattga agataaaata tttgggaaaa
541 cctatcggag gaaggcaagc ctccctaact tgagccacgt aattgaagat ctaattttag
601 gagcatctgc tgtagagcct caaataacac aagagcgccc cctcacaaat aaactaaagc
661 ggaaaaggag aggtacatc
```

It is appreciated that each of the genetic modifications as disclosed may be present separately, however, it is also appreciated that the genetic modifications are combined in the pig model of the present invention. Thus, the genetically modified pig according to the present invention harbors the mutation, wherein at least one codon of the endogenous BRCA1 gene or part thereof is mutated as described herein may be combined with the modification of the BRCA2 gene, wherein all or part of exon 11 of the porcine BRCA2 gene is deleted by homologous recombination of a construct containing a selection gene inside exon 11 sequence of BRCA2 gene into the endogenous BRCA2 gene as described herein; optionally the genetically modified pig with combined mutations further comprises the deletion of exon 11 of the endogenous porcine BRCA1 gene as described herein. It is also within the scope of the present invention that the genetically modified pig comprises the mutation, wherein exon 11 of the endogenous porcine BRCA1 is deleted and wherein all or part of exon 11 of the porcine BRCA2 gene is deleted as described herein.

Furthermore in another embodiment, the modified pig, embryo, blastocyst, donor cell and/or fetus of the present invention comprises the transcriptional product or part thereof and/or the translational product or part thereof of the porcine BRCA1 and/or BRCA2 genes as described above.

### Mitochondria Related Protein Folding Disorders

In one embodiment of the present invention the transgenic pig, embryo, blastocyst, donor cell and/or fetus is transgenic for at least one gene selected from the rat ornithicin transcabamylase (OTC) gene or part thereof, and/or the porcine OTC gene or part thereof, and/or the human OTC gene or part thereof, and/or combinations thereof. In a preferred embodiment the rat, and/or human and/or porcine OTC gene lacks the carbamyl phosphate-binding domain. It is appreciated that the cDNA or part thereof of the rat OTC gene and/or the cDNA or part thereof of the human OTC gene and/or the cDNA or part thereof of the porcine OTC gene, and/or combinations as outlined herein is within the scope of the present invention, as are the cDNA or part thereof of the rat OTC gene and/or the human OTC gene and/or porcine OTC gene, lacking the carbamyl phosphate-binding domain.

Furthermore in another embodiment, the modified pig, embryo, blastocyst, donor cell and/or fetus of the present invention comprises the transcriptional product or part thereof and/or the translational product or part thereof of the rat, porcine and/or human OTC gene.

### Epidermolysis Bullosa Simplex

In one embodiment of the present invention the genetically modified pig, embryo, blastocyst, donor cell and/or fetus is transgenic for at least one gene selected from the modified porcine keratin 14 gene or part thereof, or modified human keratin 14 gene or part thereof.

It is appreciated that the modified cDNA or part thereof of the modified porcine keratine 14 gene or the modified cDNA or part thereof of the modified human keratine 14 gene is within the scope of the present invention. Furthermore in another embodiment, the modified pig, embryo, blastocyst, donor cell and/or fetus comprises the transcriptional product or part thereof and/or the translational product or part thereof of the modified porcine and/or modified human keratin 14 gene.

### Sequence Identity

Functional equivalents and variants are used interchangeably herein. In one preferred embodiment of the invention there is also provided variants of the modified human and/or modified porcine keratin 14 gene and variants of fragments thereof, and/or variants of the mutated porcine BRCA1 and/or BRCA2 gene, and/or variants of the rat, human and/or porcine OTC gene. When being polypeptides, variants are determined on the basis of their degree of identity or their homology with a predetermined amino acid sequence, said predetermined amino acid sequence specified elsewhere herein, or, when the variant is a fragment, a fragment of any of the aforementioned amino acid sequences, respectively.

Accordingly, variants preferably have at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the predetermined sequence.

The following terms are used to describe the sequence relationships between two or more polynucleotides: “predetermined sequence”, “comparison window”, “sequence identity”, “percentage of sequence identity”, and “substantial identity”.

A “predetermined sequence” is a defined sequence used as a basis for a sequence comparison; a predetermined sequence may be a subset of a larger sequence, for example, as a segment of a full-length DNA or gene sequence given in a sequence listing, such as a polynucleotide sequence specified elsewhere herein, or may comprise a complete DNA or gene sequence. Generally, a predetermined sequence is at least 20 nucleotides in length, frequently at least 25 nucleotides in length, and often at least 50 nucleotides in length.

Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a predetermined sequence of at least 20 contiguous nucleotides and wherein the portion of the polynucleotide sequence in the comparison window may comprise additions or deletions (i.e., gaps) of 20 percent or less as compared to the predetermined sequence (which does not comprise additions or deletions) for optimal alignment of the two sequences.

Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a predetermined sequence over a comparison window of at least 20 nucleotide positions, frequently over a window of at least 25-50 nucleotides, wherein the percentage of sequence identity is calculated by comparing the predetermined sequence to the polynucleotide sequence which may include deletions or additions which total 20 percent or less of the predetermined sequence over the window of comparison. The predetermined sequence may be a subset of a larger sequence, for example, as a segment of the full-length Keratin 14 polynucleotide sequence illustrated herein.

Sequence identity is determined in one embodiment by utilising fragments of human and/or porcine keratin 14 and/or variants of porcine BRCA1 and/or BRCA2 and/or variants of rat, human and/or porcine OTC peptides comprising at least 25 contiguous amino acids and having an amino acid sequence which is at least 80%, such as 85%, for example 90%, such as 95%, for example 96%, such as 97%, for example 98%, such as 99% identical to the amino acid sequences, as defined herein, wherein the percent identity is determined with the algorithm GAP, BESTFIT, or FASTA in the Wisconsin Genetics Software Package Release 7.0, using default gap weights.

By the term “transcriptional or translational products” is meant herein products of gene transcription, such as a RNA transcript, for example an unspliced RNA transcript, a mRNA transcript and said mRNA transcript splicing products, and products of gene translation, such as polypeptide(s) translated from any of the gene mRNA transcripts and various products of post-translational processing of said polypeptides, such as the products of post-translational proteolytic processing of the polypeptide(s) or products of various post-translational modifications of said polypeptide(s).

As used herein, the term “transcriptional product of the gene” refers to a pre-messenger RNA molecule, pre-mRNA, that contains the same sequence information (albeit that U nucleotides replace T nucleotides) as the gene, or mature messenger RNA molecule, mRNA, which was produced due to splicing of the pre-mRNA, and is a template for translation of genetic information of the gene into a protein.

### Phenotypes

### Breast Cancer

The phenotypes associated with breast cancer are many. It is appreciated that the pig model of the present invention expresses at least one phenotype associated with breast cancer, such as three, for example four, five, six, seven, eight, nine, ten, eleven, 12, 13, 14, 15, 16, 17, 18, 19 or 20 phenotypes associated with breast cancer.

The phenotypes associated with breast cancer comprise unilateral breast cancer, bilateral breast cancer, secondary tumours for example in the lymph nodes in the axilla, or secondary tumours for example in liver or lung. The term secondary tumour is used to describe tumours which are not the primary tumour but are tumours that have developed by metastasis from the primary tumour or a secondary tumour. By primary tumour is meant the original site where cancer occurs. The present invention pertains to pigs of both sexes. In a particular embodiment the pig is a sow.

The present invention relates to breast cancer of any type. The breast cancer may be an adenoma, an adenocarcinoma, a carcinoma or carcinoma in situ.

The term “tumour,” as used herein, refers to all neoplastic cell growth and proliferation, whether malignant or benign, and all pre-cancerous and cancerous cells and tissues.

An adenoma is a benign tumour arising in glandular epithelium. The glandular epithelium is a type of epithelial tissue whose primary function is secretion, and is the prominent tissue forming endocrine and exocrine glands, for example in the breast. An adenoma may progress or transform into a malignant tumour which is then characterised as an adenocarcinoma. A carcinoma is defined as a malignant tumour that begins in the lining layer (epithelial cells) of organs. Carcinoma have a tendency to infiltrate into adjacent tissue and spread (metastasize) to distant organs, such as bone, liver, lung, or the brain. The present invention also relates to individuals suffering from breast cancer in the form of carcinoma in situ (CIS) which is an early form of carcinoma and is defined by the absence of invasion of surrounding tissues. In other words, carcinoma in situ is the abnormal growth of cells that proliferate in their normal habitat, hence the name ‘in situ’. Carcinoma in situ is also equivalent to the term high grade dysplasia.

The breast cancer of the present invention may be invasive or non-invasive. By invasive cancer is meant cancer characterized by spreading from its point of origination into other tissues and organs. For example, invasive breast cancers develop in milk glands (lobules) or milk passages (ducts) and spread to the nearby fatty breast tissue. Some invasive cancers spread to distant areas of the body (metastasize), but others do not. Invasive cancer is also referred to as infiltrating cancer. By analogy, the non-invasive cancers do not invade surrounding tissue.

The breast cancer from which an individual according to the present invention suffers may thus be selected from the group consisting of a primary malignant tumour, a ductal carcinoma, a lobular carcinoma, a ductal carcinoma in situ, lobular carcinoma in situ, and a secondary tumour for example in the axil, lung or liver.

One embodiment of the present invention relates to individuals suffering from invasive ductal carcinoma, a cancer that starts in the milk passages (ducts) of the breast and then breaks through the duct wall, where it invades the fatty tissue of the breast. When the cancer reaches this point, it has the potential to spread (metastasize) elsewhere in the breast, as well as to other parts of the body through the bloodstream and lymphatic system. Invasive ductal carcinoma is the most common type of breast cancer, accounting for about 80% of breast malignancies—in humans.

Another embodiment of the present invention relates to individuals suffering from ductal carcinoma in situ. Ductal carcinoma in situ is characterized as proliferation of abnormal cells within the milk passages (ducts) but where no visible signs of invasion into the duct wall are evident. This is a highly curable form of breast cancer that is treated with surgery or surgery plus radiation therapy.

The present invention also relates to Lobular carcinoma which is a cancer that begins in the lobules (the glands that make milk) of the breast. Lobular carcinoma in situ (LCIS) is a condition in which abnormal cells are found only in the lobules. When cancer has spread from the lobules to surrounding tissues, it is invasive lobular carcinoma. LCIS does not become invasive lobular carcinoma very often, but having LCIS in one breast increases the risk of developing invasive cancer in either breast.

### Mitochondria Related Protein Folding Disorders

The phenotypes associated with mitochondria related protein folding disorders are many. It is appreciated that the pig model of the present invention expresses at least one phenotype associated with mitochondria related protein folding disorders, such as three, for example four, five, six, seven, eight, nine, ten, eleven, 12, 13, 14, 15, 16, 17, 18, 19 or 20 phenotypes associated with mitochondria related protein folding disorders.

The phenotypes associated with mitochondria related protein folding disorders comprise the phenotypes observed when the pig suffers from Alzheimer's disease, Parkinson's disease or Huntington's disease.

The phenotypes associated with Alzheimer's comprise short term memory loss which progresses from seemingly simple and often fluctuating forgetfulness to a more pervasive loss of short-term memory, then of familiar and well-known skills or objects. In humans, loss of memory is often followed by aphasia and disorientation. Alzheimer's disease may also include behavioral changes, such as outbursts of violence or excessive passivity in people/pigs having no previous history of such behavior. In the later stages of the disease deterioration of musculature and mobility is observed.

The diagnosis is made primarily on the basis of clinical observation and tests of memory and intellectual functioning over a series of weeks or months. No medical tests are available to diagnose Alzheimer's disease conclusively pre-mortem. However, Alzheimer's disease can now be diagnosed by experts skilled in memory disorders with high accuracy. Functional neuroimaging studies such as positron emission tomography (PET) and single photon emission computed tomography (SPECT) scans can provide a supporting role.

According to the present invention the at least one expressed phenotype of the porcine model of Alzheimer's disease may include the following parameters to be observed at 6, 12, 18, 24 months of age:

### Biochemistry

Transgene (APP or PS1) mRNA detection by Northern blotting, RT-PCR, in situ RNA hybridisation to cryostat brain sections.

Transgene protein detection by Western blotting, immunohistochemistry on paraffin embedded brain sections, sandwich ELISA for detection of A βin cerebro-spinal fluid.

### Neuropathology

H+E and Bielchowsky staining of brain sections to detect specific AD pathology (amyloid plaques and neurofibrillary tangles). Immunohistochemistry to detect A, βtau, ubiquitin. Diagnosis according to standardized neuropathological criteria for Alzheimer's disease (Reagan criteria with CERAD score and Braak & Braak stadium).

### Behavioral Analysis

Base-line studies and validation of the following tests:
Spatial memory of the pigs is tested in an eight-room labyrinth (central hall-way with 4 rooms on each side). In 4 to 6 rooms is placed reward. The animal learns in which rooms rewards never occur and remember which rooms it has visited.
Object recognition test takes place in an arena where two identical objects are presented to the pig for a well defined period of time. The pig is removed from the arena in a delay-period while one of the familial objects is substituted by a new object. The pig returns to the arena and the time the pig uses to explore the known and unknown objects is measured.
Olfaction is tested in an olfactometer where the animal is presented to an odorant in various concentrations (+stimulus) or is presented to air without odorant (−stimulus). The animal has already been trained, by operant conditioning, to press a pedal (A) when +stimulus and a pedal (B) if −stimulus. When threshold for detection is reached the pig will perform a −response (press pedal B) in spite of the presence of a small amount of odorant.
   Brain Imaging, such as PET and MRI Studies

The phenotypes associated with Parkinson's disease comprise motor-symptoms and non-motor symptoms. Non-motor symptoms are for example the occurrence of depression, slowed reaction time and difficulties in differential allocation of attention, impulse control, set shifting, prioritizing, evaluating the salience of ambient data, interpreting social cues, and subjective time awareness which may in humans for example lead to dementia. Symptoms such as short time memory loss, disturbances in sleep such as insomnia and somnolence at daytime.

Motor-symptoms are symptoms that affect movement, for example tremor which is increased when the limb is resting and decreased due to voluntary movement. Slowness or even absence of movement for example also combined with rapid movements which are repeated is another example of a motor-symptom of Parkinson's disease. Balance disorders such as those that occur due to failure of reflexes which may lead to impaired balance and falls are examples of motor symptoms. In addition, stiffness or increased muscle tone, also in combination with resting tremor, is an example of motor symptoms associated with Parkinson's disease. Gait, in which the feet are not lifted from the ground, forward-flexed posture and decreased arm swing (as observed in humans), fatigue are also examples of motor symptoms due to Parkinson's disease.

The phenotypes associated with Huntington's disease comprise psychopathological, physical and/or cognitive symptoms. Cognitive symptoms varies considerable but symptoms such as anxiety, depression, aggressive behaviour are often observed in Huntington's disease. The physical symptoms comprise the characteristic chorea which are uncontrollable, jerky, random, rapid movements, which tend gradually to increase as the disease progresses, which leads to a general lack of coordination and an unsteady gait. The cognitive symptoms associated with Huntington's disease are in humans for example impaired executive function (planning; cognitive flexibility, abstract thinking, rule acquisition, initiating appropriate actions, and inhibiting inappropriate actions). But also perceptual and spatial skills of the patient and his surroundings are impaired, but also the ability for example to learn new skills is impaired.

### Epidermolysis Bullosa Simplex

The phenotypes associated with epidermolysis bullosa simplex are many. It is appreciated that the pig model of the present invention expresses at least one phenotype associated with epidermolysis bullosa simplex, such as three, for example four, five, six, seven, eight, nine, ten, eleven, 12, 13, 14, 15, 16, 17, 18, 19 or 20 phenotypes associated with epidermolysis bullosa simplex.

The phenotypes associated with epidermolysis bullosa simplex comprise the disease appearance selected from skin blisters on the hands, on the feet or spread over the entire body also as ring formed blisters. A blister occurs when the epidermis layer of the skin separates from the dermis (fibre layer), a pool of lymph and other bodily fluids collects between these layers while the skin will re-grow from underneath. In one embodiment the phenotype is as observed in Weber Cockayne, or as in Köbner, or as in Dowling Meara Epidermolysis Bullosa Simplex. The phenotypes as observed in Weber Cockayne Epidermolysis Bullosa Simplex are relatively mild, in which blisters rarely extend beyond the feet and hands. Blisters may not become evident until the child begins to walk. The phenotypes as observed in Köbner Epidermolysis Bullosa Simplex, blistering may be obvious from birth, or develop during the first few weeks of life. Blistering occurs in areas where friction is caused by clothing, or for example the edges of a nappy. Often blisters are found inside the mouth. In the phenotypes as observed in Dowling Meara severe blistering appears already during or shortly after birth. Blisters may develop in cluster, and spread like rings.

Methods for Producing Pig Model for Studying Breast Cancer, Mitochondria Related Protein Folding Disorders and/or Epidermolysis Bullosa Simplex

The present invention provides improved procedures for cloning mammals by nuclear transfer which refers to the introduction of a full complement of nuclear DNA from one cell to an enucleated cell. The genetically modified pig of the present invention may be produced using any technique in which modified genetic material, transcriptional product and/or translational product or part thereof is transferred from at donor cell to a host cell, such as an enucleated oocyte. A number of techniques exist such as introducing genetic material from a genetically modified somatic cell into an enucleated oocyte by for example microinjection or by nuclear transfer

In cloning, the transfer of the nucleus of a somatic (body) cell or somatic cell into an egg cell (oocyte) which has had its own nucleus removed (denucleated or enucleated) is called somatic cell nuclear transfer. The new individual will develop from this reconstructed embryo and be genetically identical to the donor of the somatic cell. In the present invention a modified pig, porcine embryo, blastocyst and/or fetus model is obtainable by somatic cell nuclear transfer comprising the steps of a) establishing at least one oocyte having at least a part of a modified zona pellucida, b) separating the oocyte into at least two parts obtaining at least one cytoplast, c) establishing a donor cell or cell nucleus having desired genetic properties, d) fusing at least one cytoplast with the donor cell or membrane surrounded cell nucleus, e) obtaining a reconstructed embryo, f) activating the reconstructed embryo to form an embryo; and g) transferring saod cultured embryo to a host mammal such that the embryo develops into a genetically modified fetus, wherein said genetically modified embryo obtainable by nuclear transfer comprises steps a) to e) and/or f),

wherein said genetically modified blastocyst obtainable by nuclear transfer comprises steps a) to e) and/or f), wherein said genetically modified fetus obtainable by nuclear transfer comprises steps a) to g).

It is appreciated that the donor cell or cell nucleus of c) harbours genetic determinants for breast cancer, mitochondria related protein folding disorders and/or epidermolysis bullosa simplex, for example in the form of variants of the modified human and/or modified porcine keratin 14 gene and variants of fragments thereof, and/or variants of the mutated porcine BRCA1 and/or BRCA2 gene, and/or variants of the rat, human and/or porcine OTC gene and/or transcriptional and/or translational products thereof. The host mammal of g) is in one embodiment a pig, preferably a Goettingen mini pig.

However, the present invention also relates to a method for producing a transgenic pig, porcine blastocyst, embryo and/or fetus as a model for breast cancer, mitochondria related protein folding disorders and/or epidermolysis bullosa simplex comprising the steps of a) establishing at least one oocyte, b) separating the oocyte into at least three parts obtaining at least two cytoplasts, c) establishing a donor cell or cell nucleus having desired genetic properties, d) fusing at least one cytoplast with the donor cell or membrane surrounded cell nucleus, e) obtaining a reconstructed embryo f) activating the reconstructed embryo to form an embryo; and g) transferring saod cultured embryo to a host mammal such that the embryo develops into a genetically modified fetus, wherein said genetically modified embryo obtainable by nuclear transfer comprises steps a) to e) and/or f), wherein said genetically modified blastocyst obtainable by nuclear transfer comprises steps a) to e) and/or f), wherein said genetically modified fetus obtainable by nuclear transfer comprises steps a) to g).

The oocyte of b) may in another embodiment be separated into at least three parts obtaining at least two cytoplasts. It is appreciated that the donor cell or cell nucleus of c) harbours genetic determinants for breast cancer, mitochondria related protein folding disorders and/or epidermolysis bullosa simplex, for example in the form of variants of the modified human and/or modified porcine keratin 14 gene and variants of fragments thereof, and/or variants of the mutated porcine BRCA1 and/or BRCA2 gene, and/or variants of the rat, human and/or porcine OTC gene and/or transcriptional and/or translational products thereof. The host mammal of g) is in one embodiment a pig, preferably a Goettingen mini pig.

The various parameters are described in detail below.

### Oocyte

The term ‘oocyte’ according to the present invention means an immature female reproductive cell, one that has not completed the maturing process to form an ovum (gamete). In the present invention an enucleated oocyte is the recipient cell in the nuclear transfer process.

The oocytes according to the present invention are isolated from oviducts and/or ovaries of a mammal. Normally, oocytes are retrieved from deceased pigs, although they may be isolated also from either oviducts and/or ovaries of live pigs. In one embodiment the oocytes are isolated by oviductal recovery procedures or transvaginal recovery methods. In a preferred embodiment the oocytes are isolated by aspiration. Oocytes are typically matured in a variety of media known to a person skilled in the art prior to enucleation. The oocytes can also be isolated from the ovaries of a recently sacrificed animal or when the ovary has been frozen and/or thawed. Preferably, the oocytes are freshly isolated from the oviducts.

Oocytes or cytoplasts may also be cryopreserved before use. While it will be appreciated by those skilled in the art that freshly isolated and matured oocytes are preferred, it will also be appreciated that it is possible to cryopreserve the oocytes after harvesting or after maturation. If cryopreserved oocytes are utilised then these must be initially thawed before placing the oocytes in maturation medium. Methods of thawing cryopreserved materials such that they are active after the thawing process are well-known to those of ordinary skill in the art. However, in general, cryopreservation of oocytes and cytoplasts is a very demanding procedure, and it is especially difficult in pigs, because of the above mentioned general fragility of pig oocytes and cytoplasts, and because of the high lipid content that makes them very sensitive to chilling injury (i.e. injury that occurs between +15 and +5° C. during the cooling and warming procedure).

In another embodiment, mature (metaphase II) oocytes that have been matured in vivo, may be harvested and used in the nuclear transfer methods disclosed herein. Essentially, mature metaphase II oocytes are collected surgically from either nonsuperovulated or superovulated pigs 35 to 48 hours past the onset of estrus or past the injection of human chorionic gonadotropin (hCG) or similar hormone.

Where oocytes have been cultured in vitro, cumulus cells that are surrounding the oocytes in vivo may have accumulated may be removed to provide oocytes that are at a more suitable stage of maturation for enucleation. Cumulus cells may be removed by pipetting or vortexing, for example, in the presence of in the range of 0.1 to 5% hyaluronidase, such as in the range of 0.2 to 5% hyaluronidase, for example in the range of 0.5 to 5% hyaluronidase, such as in the range of 0.2 to 3% hyaluronidase, for example in the range of 0.5 to 3% hyaluronidase, such as in the range of 0.5 to 2% hyaluronidase, for example in the range of 0.5 to 1% hyaluronidase, such as 0.5% hyaluronidase.

The first step in the preferred methods involves the isolation of a recipient oocyte from a suitable pig. In this regard, the oocyte may be obtained from any pig source and at any stage of maturation.

The stage of maturation of the oocyte at enucleation and nuclear transfer has been reported to be of significance for the success of nuclear transfer methods. Immature (prophase I) oocytes from pig ovaries are often harvested by aspiration. In order to employ techniques such as genetic engineering, nuclear transfer and cloning, such harvested oocytes are preferably matured in vitro before the oocyte cells may be used as recipient cells for nuclear transfer.

Preferably, successful pig embryo cloning uses the metaphase II stage oocyte as the recipient oocyte because it is believed that at this stage of maturation the oocyte can be or is sufficiently activated to treat the introduced nucleus as if it were a fertilising sperm. However, the present invention relates to any maturation stage of the oocyte which is suitable for carrying out somatic cell nuclear transfer, embryos, blastocysts, and/or transgenic pigs obtainable by the method of somatic cell nuclear transfer of the present invention.

The in vitro maturation of oocytes usually takes place in a maturation medium until the oocyte has reached the metaphase II stage or has extruded the first polar body. The time it takes for an immature oocyte to reach maturation is called the maturation period.

In a preferred embodiment of the present invention the oocyte is from sow or gilt, preferably from a sow.

The donor (somatic cell or nucleus of somatic cell) and recipient (cytoplast) involved in the cell nuclear transfer method according to the present invention is a pig. Likewise, reconstructed embryos may be implanted in a pig according to the present invention. The different pigs suitable as donor, recipient or foster mother are described elsewhere herein.

The donor pig according to the present invention may be female, or male. The age of the pig can be any age such as an adult, or for example a fetus.

### Embryo

According to the present invention a reconstructed embryo (i.e. single cell embryo) contains the genetic material of the donor cell. Subsequently, the reconstructed embryo divides progressively into a multi-cell embryo after the onset of mitosis. In vitro the onset of mitosis is typically induced by activation as described herein.

In the present invention the term ‘embryo’ also refers to reconstructed embryos which are embryos formed after the process of nuclear transfer after the onset of mitosis by activation. Reconstructed embryos are cultured in vitro.

When the embryo contains about 12-16 cells, it is called a “morula”. Subsequently, the embryo divides further and many cells are formed, and a fluid-filled cystic cavity within its center, blastocoele cavity. At this stage, the embryo is called a “blastocyst”. The developmental stage of the “fertilized” oocyte at the time it is ready to implant; formed from the morula and consists of an inner cell mass, an internal cavity, and an outer layer of cells called trophectodermal cells.

The blastocyst according to the present invention may be implanted into the uterus of a host mammal and continues to grow into a fetus and then an animal.

In the methods provided herein for producing genetically modified or transgenic non-human mammal, for cloning a non-human mammal, for culturing a reconstructed embryo, and/or for cryopreservation of a pig embryo, the embryo may be cultured in vitro. The embryo may for example be cultured in sequential culture. It will be appreciated that the embryo may be a normal embryo, or a reconstructed embryo as defined elsewhere herein.

The present invention thus relates to a modified porcine embryo, blastocyst and/or fetus derived from the genetically modified pig model as disclosed herein and/or the modified porcine embryo comprises at least one modified exon 3 or part thereof of the BRCA1 gene and/or porcine BRCA1 comprising a nucleotide substitution from T to G resulting in amino acid substitution from Cys to Gly at codon 61 of exon 3 and/or exon 11 or part thereof of the BRCA1 gene and/or porcine BRCA1 gene comprising a deletion of at least one allele of exon 11 or part thereof of the BRCA1 gene and/or exon 11 or part thereof of the BRCA2 gene, and/or porcine BRCA2 gene comprising a deletion of at least one allele of exon 11 or part thereof of the BRCA2 gene and/or rat Ornithine TransCarbamylase (OTC) gene or part thereof, and/or human Ornithine TransCarbamylase gene or part thereof, and/or porcine Ornithine TransCarbamylase gene or part thereof, and/or rat Ornithine TransCarbamylase cDNA or part thereof, and/or porcine Ornithine TransCarbamylase cDNA or part thereof, and/or human Ornithine TransCarbamylase cDNA or part thereof, and/or porcine keratin 14 gene or part thereof, and/or human keratin 14 gene or part thereof, and/or porcine keratin 14 cDNA or part thereof, and/or human keratin 14 cDNA or part thereof, and/or a transcriptional and/or translational product thereof, separately or in combination as described in detail herein.

It is appreciated that the modified porcine embryo, blastocyst and/or fetus derivable from the modified pig model for studying breast cancer, mitochondria related protein folding disorders and/or epidermolysis bullosa simplex, expressing at least one phenotype associated with breast cancer, mitochondria related protein folding disorders and/or epidermolysis bullosa simplex may have been the result of the crossing of for example a pig transgenic for at least any variants of the modified human and/or modified porcine keratin 14 gene and/or fragments thereof, and/or variants of the mutated porcine BRCA1 and/or BRCA2 gene, and/or variants of the rat, human and/or porcine OTC gene.

### Cytoplast

An oocyte or a part of an oocyte from which the nucleus has been removed.

### Donor Cell

By the term ‘donor cell’ of the present invention is meant somatic cell and/or cells derived from the germ line.

By the term ‘somatic cell’ of the present invention is meant any (body) cell from an animal at any stage of development. For example somatic cells may originate from fetal, neonatal or adult tissue. Especially preferred somatic cells are those of foetal or neonatal origin. However, cells from a germ line may also be used. According to the present invention a donor cell is a somatic cell. In another embodiment of the present invention the donor cell is a cell derived from a germ cell line.

In a preferred embodiment of the present invention the donor cell harbours desired genetic properties. However, the donor cell may harbour desired genetic properties which have been gained by genetic manipulation as described elsewhere herein.

Somatic cells are selected from the group consisting of epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells.

These may be obtained from different organs, e.g., skin, lung, pancreas, liver, stomach, intestine, heart, reproductive organs, bladder, kidney, urethra and other urinary organs.

The pigs from which the somatic cells may be derived are described elsewhere herein. A preferred embodiment of the invention is the use of somatic cells originating from the same species as the recipient oocyte (cytoplast).

Preferably, the somatic cells are fibroblast cells as the can be obtained from both developing fetuses, newborn piglets and adult animals in large quantities. Fibroblasts may furthermore be easily propagated in vitro. Most preferably, the somatic cells are in vitro cultured fibroblasts of foetal or neonatal origin.

In a preferred embodiment the somatic cells are modified. In yet a further preferred embodiment of the present invention the somatic cells are preferably of foetal or neonatal origin, or for example from adults.

One aspect of the present invention relates to a genetically modified donor cell and/or cell nucleus derived from the genetically modified pig model as disclosed herein, and/or a genetically modified donor cell and/or cell nucleus being transgenic due to insertion of at least one modified exon 3 or part thereof of the BRCA1 gene and/or porcine BRCA1 comprising a nucleotide substitution from T to G resulting in amino acid substitution from Cys to Gly at codon 61 of exon 3 and/or exon 11 or part thereof of the BRCA1 gene and/or porcine BRCA1 gene comprising a deletion of at least one allele of exon 11 or part thereof of the BRCA1 gene and/or exon 11 or part thereof of the BRCA2 gene, and/or porcine BRCA2 gene comprising a deletion of at least one allele of exon 11 or part thereof of the BRCA2 gene and/or rat Ornithine TransCarbamylase (OTC) gene or part thereof, and/or human Ornithine TransCarbamylase gene or part thereof, and/or porcine Ornithine TransCarbamylase gene or part thereof, and/or rat Ornithine TransCarbamylase cDNA or part thereof, and/or porcine Ornithine TransCarbamylase cDNA or part thereof, and/or human Ornithine TransCarbamylase cDNA or part thereof, and/or porcine keratin 14 gene or part thereof, and/or human keratin 14 gene or part thereof, and/or porcine keratin 14 cDNA or part thereof, and/or human keratin 14 cDNA or part thereof, and/or a transcriptional and/or translational product thereof, separately or in combination as described in detail herein. It is appreciated that the genetically modified donor cell may be any type of tissue as described elsewhere herein, however, the preferred donor cell is a porcine fibroblast cell.

It is appreciated that the genetically modified porcine donor cell or cell nucleus derivable from the genetically modified pig model for studying breast cancer, mitochondria related protein folding disorders and/or epidermolysis bullosa simplex, expressing at least one phenotype associated with breast cancer, mitochondria related protein folding disorders and/or epidermolysis bullosa simplex may have been the result of the crossing of for example a pig transgenic for at least one variant of the modified human and/or modified porcine keratin 14 gene and variants of fragments thereof, and/or variants of the mutated porcine BRCA1 and/or BRCA2 gene, and/or variants of the rat, human and/or porcine OTC gene.

### Type of Genetic Modification

The donor cells may be genetically modified by any of standard method known in the art. The genetic modification may be a modification of the genomic DNA by deletion, insertion, duplication and/or other forms of mutation, including point mutation. The modification may be made in coding sequences and/or non-coding sequences. DNA constructs for insertion may harbour a gene of interest and/or regulatory sequences such as promoters, insulators, enhancers, repressors or ribosomal entry sites.

In some embodiments, only one genetic modification is introduced in the genome. In other embodiments, however, the genome may be modified at more than one site. Suitable techniques for genetic modification of mammalian cells, such as fibroblasts, include techniques such as gene addition by nonhomologous recombination, gene replacement by homologous recombination, and gene editing. This may include the use of retroviral insertion, transposon transfer and/or artificial chromosome techniques. Nonhomologous DNA recombination may e.g. be carried out as described in Kragh et al. (2004) Reprod. Fert. Dev. 16:290 or Kragh et al. (2004) Reprod. Fert. Dev. 16:315, Transposon-based gene transfer may be carried out as described in Izsvak et al. (1997) Cell 91:501. Gene replacement by homologous recombination may e.g. involve the techniques described by Urnow et al. (2005) Nature 435:646. Techniques for gene editing have been described in Andersen et al. (2002) J. Mol. Med. 80:770, Liu et al (2002) Gene Ther. 9:118 and Sørensen et al. (2005) J. Mol. Med. 83:39.

In a preferred embodiment the donor cell is genetically modified by random integration of the genes disclosed herein into the genome of the donor cell.

In another preferred embodiment of the present invention the donor cell is genetically modified (as described in a copending application). The donor cell or nucleus carries a SB tagged genome containing a Flp recombination target site for site specific gene insertion or integration. The SB tagged genome result from the integration of a recombinant target vector comprising a DNA transposon construct and a bicistronic gene cassette comprising (i) a FRT recombination site and (ii) an IRES-driven selection gene. The DNA transposon construct may be any construct in which any DNA transposon is present. In the present invention the DNA transposon construct is the Sleeping Beauty (SB) DNA transposon vector. The FRT recombination site may be embedded in the coding sequence of a selection gene which allows for detecting whether a transposition has occurred. The selection gene of the present invention is not limited to any particular selection gene. In preferred embodiments the selection gene are genes conferring resistance to antibiotics or drugs, such as puromycin, tetracycline, streptomycin or hygromycin resistance genes, or the enhanced green fluorescent protein (eGFP) gene, red fluorescent protein genes or the like. The FRT recombination site may thus be embedded in a SV40 promoter driven fusion variant of the selection gene. However, any promoter suitable for conferring expression of a selection gene may be used according to the present invention. Non-limiting examples of such promoters are CMV (cytomegalovirus) or PGK promoter.

The IRES-driven selection gene is similarly not limited to any particular selection gene. In preferred embodiments the selection gene are genes conferring resistance to antibiotics or drugs, such as puromycin, tetracycline, streptomycin or hygromycin resistance genes, or the enhanced green fluorescent protein (eGFP) gene, red fluorescent protein genes or the like.

The recombinant vector construct may also comprise at least one site for Cre recombinase. The at least one site for Cre recombinase may be located as disclosed in the examples herein.

The donor cell or nucleus may also originate from a genetically modified pig comprising at least one site for integration of at least one transgene. A preferred embodiment is a donor cell or nucleus in the form of a fibroblast, such as a primary fibroblast.

The present invention also relates to a method for producing a porcine cell comprising a SB tagged genome containing a Flp recombination target site for site-specific gene insertion. The method comprises the steps of

a) providing a mammalian cell, b) transfecting the cell of a) with a plasmid expressing a transposase and a recombinant target vector comprising a DNA transposon construct and a bicistronic gene cassette comprising (i) a FRT recombination site and ii) an IRES-driven selection gene, c) selecting SB tagged cells.

As described elsewhere herein the mammalian cell may be any cell. In one embodiment in which the porcine cell is subsequently to be used for producing a genetically modified pig by nuclear transfer according to the hand-made protocol as described herein, the porcine cell is in a preferred embodiment a fibroblast and most preferred a porcine primary fibroblast.

It is appreciated that a desired transgene may be integrated directly into the at least one site for integration present in the genome of the cell. However, the cell in which the genome carries the at least one site for integration is in another embodiment used as a donor cell for the production of a genetically modified pig by for example microinjection of the donor cell or nucleus thereof into a oocyte or by for example somatic nuclear transfer. In a preferred embodiment the donor cell or the nucleus thereof is used for the production of a genetically modified pig by somatic nuclear transfer using the procedure as described elsewhere herein.

The transgene or gene of interest to be integrated in the targeted cells of the present invention is not limited to any particular gene. In one embodiment the gene to be integrated is a disease-causing gene which results in the formation of a genetically modified pig displaying a phenotype of interest. According to the present invention the gene of interest to be integrated into the porcine cell is at least one variant of the modified human and/or modified porcine keratin 14 gene and/or variants of fragments thereof, and/or variants of the mutated porcine BRCA1 and/or BRCA2 gene, and/or variants of the rat, human and/or porcine OTC gene, as described elsewhere herein.

The integration of the transgene into the at least one site for integration present in the genome of the cell is employed by transfection into the cell of plasmid DNA containing the gene of interest and also FRT sites, and a plasmid expressing the Flp-recombinase used to support integration at the FRT sites.

### Enucleation

The method of enucleation of an oocyte may be selected from the group of methods consisting of aspiration, physical removal, use of DNA-specific fluorochromes, exposure to ultraviolet light and/or chemically assisted enucleation. In one embodiment the present invention relates to the use of DNA-specific fluorochromes.

Enucleation may, however, be performed by exposure with ultraviolet light. In a particular embodiment enucleation is chemically assisted prior to physical removal of the nucleus. Chemically assisted enucleation using for example antineoplastic agents, such as demecolcine (N-deacetyl-N-methyl 1 colchicine), and/or for example etoposide or related agents may be performed prior to enzymatic modification of zona pellucida. Chemically assisted enucleation comprises culturing matured COCs in maturation medium as described elsewhere herein supplemented with demecolcine for a particular period of time. In the range of 0.1 μg/ml to 10 μg/ml demecolcine, such as 0.2 μg/ml to 10 μg/ml, for example 0.3 μg/ml to 10 μg/ml, such as 0.25 μg/ml to 5 μg/ml, for example 0.3 μg/ml to 1 μg/ml, such as 0.25 μg/ml to 0.5 μg/ml, for example 0.4 μg/ml demecolcin may be supplemented to the maturation medium. Similarly, maturation medium may be supplemented with etoposide for example in the range of 0.1 μg/ml to 10 μg/ml etoposide, such as 0.2 μg/ml to 10 μg/ml, for example 0.3 μg/ml to 10 μg/ml, such as 0.25 μg/ml to 5 μg/ml, for example 0.3 μg/ml to 1 μg/ml, such as 0.25 μg/ml to 0.5 μg/ml, for example 0.4 μg/ml etoposide may be supplemented to the maturation medium. The time for culturing the COCs in the presence of antineoplastic agents ranges from 10 min to 5 hrs, such as 30 minutes to 5 hrs, for example 10 minutes to 2 hrs, such as 30 min to 2 hrs, for example 10 min to 1.5 hrs, such as 20 min to 3 hrs, for example 10 min to 3 hrs, such as 30 min to 1.5 hrs, for example 45 min.

In a particular embodiment chemically assisted enucleation is performed using 0.45 μg/ml demecolcine and/or etoposide added to the maturation medium for 45 min.

In a particular embodiment it is preferred that the enucleation is by physical removal of the nucleus. The physical removal may be by separation for example by bisection of the oocyte into two halves (two parts), one which contains the nucleus and the enucleated oocyte half, known as the cytoplast, removing the nucleated half of the oocyte and selecting the resulting cytoplast for further procedures of the invention. Alternatively the separation is by trisection, resulting in three parts of which two parts are cytoplasts. In another embodiment the oocyte may be separated into four parts, resulting in the production of three cytoplasts. The oocyte may even be separated into five parts by physical removal, resulting in four cytoplasts. Similarly, the oocyte may be separated into six parts, for example seven parts, such as eight parts, for example nine parts, such as ten or more parts.

The physical separation of the oocyte and subsequent removal of the nucleus-bearing part of the oocyte may be achieved by the use of a microsurgical blade.

The oocytes may be screened to identify which oocytes have been successfully enucleated. Oocyte parts that harbour nuclear DNA may be identified by staining with Hoechst fluorochrome, the staining procedure of which is known to a person skilled in the art. Oocyte parts harbouring nuclear DNA are discarded and the enucleated oocytes (cytoplasts) are selected for further procedures.

### Zona Pellucida

Zona pellucida is a thick, transparent, noncellular layer or envelope of uniform thickness surrounding an oocyte

Generally, an intact zona pellucida is considered to be important in cell nuclear transfer due to a number of parameters. One parameter is to keep the polar body close to the metaphase plate of the oocyte in order to indicate the appropriate site for enucleation. Another parameter relates to the keeping of the donor cell close to the oocyte cytoplast before and during fusion. The zona is also believed to confer protection for the donor cell and cytoplast during fusion. Finally, embryo development after reconstitution and activation is believed to be supported by the zona pellucida.

Modification of at least a part of the zona pellucida can be performed by a number of methods. For example physical manipulation can be used to modify the zona. But also chemical treatment with agents such as acidic solutions (acidic Tyrode) can be employed. One example of chemical agents that can be employed in the present invention is acidic solutions, for example Tyrode. In a particular embodiment of the invention the zona pellucida is modified by enzymatic digestion. Such enzymatic digestion may be performed by enzymes comprising for example trypsin. Alternatively a specific protease may be used, such as pronase.

In a preferred embodiment the enzymatic digestion results in at least a partial digestion of a part of zona pellucida which in a preferred embodiment of the present invention means that at least a part of the zona pellucida is being removed, or that the zona pellucida is partly removed. In the present context the zona pellucida is not completely removed.

According to an especially preferred embodiment of the present invention the partially digested part of zona pellucida is characterized by the zona pellucida still being visible and by the fact that the oocyte has not become misshaped.

The partial digestion may be achieved by exposure to a protease. In another embodiment of the present invention the partial digestion may be accomplished by the use of a pronase. In yet another embodiment the partial digestion may be achieved by a combination of a protease and pronase.

In a preferred embodiment the concentration of pronase is in the range of 0.1 mg/ml to 10 mg/ml, such as 0.5 mg/ml to 10 mg/ml, for example 1 mg/ml to 10 mg/ml, such as 1.5 mg/ml to 10 mg/ml, for example 2 mg/ml to 10 mg/ml, such as 2.5 mg/ml to 10 mg/ml, for example 2.75 mg/ml to 10 mg/ml, such as 3 mg/ml to 10 mg/ml, for example 3.25 mg/ml to 10 mg/ml, such as 3.3 mg/ml to 10 mg/ml, for example 3.5 mg/ml to 10 mg/ml.

A preferred embodiment is a pronase concentration in the range of 2 mg/ml to 5 mg/ml, such as 2.25 mg/ml to 5 mg/ml, for example 2.5 mg/ml to 5 mg/ml, such as 2.75 mg/ml to 5 mg/ml, for example 2.8 mg/ml to 5 mg/ml, such as 2.9 mg/ml to 5 mg/ml, for example 3 mg/ml to 5 mg/ml, such as 3.1 mg/ml to 5 mg/ml, for example 3.2 mg/ml to 5 mg/ml, such as 3.3 mg/ml to 5 mg/ml.

A particular embodiment of the present invention is a pronase concentration in the range of 1 mg/ml to 4 mg/ml, for example 1 mg/ml to 3.9 mg/ml, such as 1 mg/ml to 3.8 mg/ml, for example 1 mg/ml to 3.7 mg/ml, such as 1 mg/ml to 3.6 mg/ml, for example 1 mg/ml to 3.5 mg/ml such as 1 mg/ml to 3.4 mg/ml, for example 1 mg/ml to 3.3 mg/ml.

In a preferred embodiment the pronase concentration is in the range of 2.5 mg/ml to 3.5 mg/ml, such as 2.75 mg/ml to 3.5 mg/ml, for example 3 mg/ml to 3.5 mg/ml. In a special embodiment the pronase concentration is 3.3 mg/ml.

It is clear to the skilled person that the pronase should be dissolved in an appropriate medium, one preferred medium according to the present invention is T33 (Hepes buffered TCM 199 medium containing 33% cattle serum (as described earlier—Vajta, et al., 2003).

The time of incubation of the oocyte in the pronase solution is in the range of 1 second to 30 seconds, such as 2 seconds to 30 seconds, for example 3 seconds to 30 seconds, such as 4 seconds to 30 seconds, such as 5 seconds to 30 seconds.

In another embodiment of the present invention the incubation time is in the range of 2 seconds to 15 seconds, such as 2 seconds to 14 seconds, for example 2 seconds to 13 seconds, such as 2 seconds to 12 seconds, for example 2 seconds to 11 seconds, such as 2 seconds to 10 seconds, for example 2 seconds to 9 seconds, such as 2 seconds to 8 seconds, for example 2 seconds to 7 seconds, such as 2 seconds to 6 seconds, for example 2 seconds to 5 seconds.

In a particular embodiment of the present invention the incubation time is in the range of 3 seconds to 10 seconds, such as 3 seconds to 9 seconds, for example 4 seconds to 10 seconds, such as 3 seconds to 8 seconds, for example 4 seconds to 9 seconds, such as 3 seconds to 7 seconds, for example 4 seconds to 8 seconds, such as 3 seconds to 6 seconds, for example 4 seconds to 7 seconds, such as 3 seconds to 5 seconds, for example 4 seconds to 6 seconds, such as 4 seconds to 5 seconds. An especially preferred incubation time is 5 seconds.

In a preferred embodiment of the present invention the oocyte is treated for 5 seconds in a 3.3 mg/ml pronase solution at 39° C.

### Reconstructed Embryo

By the term ‘reconstructed embryo’ is meant the cell which is formed by insertion of the donor cell or nucleus of the donor cell into the enucleated oocyte which corresponds to a zygote (during normal fertilisation). However, the term ‘reconstructed embryo’ is also referred to as the ‘reconstituted cell’. In the present invention the donor cell is a somatic cell. However, the donor cell may also be derived from a germ line cell.

### Fusion

The transfer of a donor cell or a membrane surrounded nucleus from a donor cell to at least cytoplast is according to the present invention performed by fusion. In the scenarios described below the term ‘donor cell’ also refers to a membrane surrounded nucleus from a donor cell. Fusion may be achieved by a number of methods.

Fusion may be between a donor cell and at least one cytoplast, such as between a donor cell and at least two cytoplasts, for example between a donor cell and at least two cytoplasts, such as between a donor cell and at least three cytoplasts, such as between a donor cell and at least four cytoplasts, for example between a donor cell and at least five cytoplasts, such as between a donor cell and at least six cytoplasts, for example between a donor cell and at least seven cytoplasts, such as between a donor cell and at least eight cytoplasts.

Fusion may be performed according to the listed combinations above simultaneously or sequentially. In one embodiment of the present invention the fusion is performed simultaneously. In another embodiment fusion of the at least one cytoplast and a donor cell is performed sequentially.

For example fusion may be achieved by chemical fusion, wherein a donor cell and the at least one cytoplast are exposed to fusion promoting agents such as for example proteins, glycoproteins, or carbohydrates, or a combination thereof. A variety of fusion-promoting agents are known for example, polyethylene glycol (PEG), trypsin, dimethylsulfoxide (DMSO), lectins, agglutinin, viruses, and Sendai virus. Preferably phytohemaglutinin (PHA) is used. However mannitol and, or polyvinylalcohol may be used.

Alternatively, fusion may be accomplished by induction with a direct current (DC) across the fusion plane. Often an alternating current (AC) is employed to align the donor and recipient cell. Electrofusion produces a sufficiently high pulse of electricity which is transiently able to break down the membranes of the cytoplast and the donor cell and to reform the membranes subsequently. As a result small channels will open between the donor cell and the recipient cell. In cases where the membranes of the donor cell and the recipient cell connect the small channels will gradually increase and eventually the two cells will fuse to one cell.

Alignment of the at least one cytoplast and the donor cell may be performed using alternating current in the range of 0.06 to 0.5 KV/cm, such as 0.1 to 0.4 KV/cm, for example 0.15 to 0.3 KV/cm. In a preferred embodiment alignment of the at least one cytoplast and the donor cell may be performed using alternating current at 0.2 KV/cm.

Fusion may be induced by the application of direct current across the fusion plane of the at least one cytoplast and the donor cell. Direct current in the range of 0.5 to 5 KV/cm, such as 0.75 to 5 KV/cm, for example 1 to 5 KV/cm, such as 1.5 to 5 KV/cm, for example 2 to 5 KV/cm. Another preferred embodiment of the present invention is the application of direct current in the range of 0.5 to 2 KV/cm. In a further preferred embodiment the direct current may be 2 KV/cm.

The direct current may preferably be applied for in the range of 1-15 micro seconds, such as 5 to 15 micro seconds, for example 5 to 10 micro seconds. A particular embodiment may be 9 micro seconds.

In an especially preferred embodiment fusion with direct current may be using a direct current of 2 KV/cm for 9 micro seconds.

Electrofusion and chemical fusion may however be also be combined.

Typically electrofusion is performed in fusion chambers as known to the skilled person.

Fusion may be performed in at least one step, such as in two steps, for example three steps, such as in four steps, for example in five steps, such as six steps, for example seven steps, such as in eight steps.

Fusion may be performed in for example a first step wherein the at least one cytoplast is fused to the donor cell. A second step of fusion may comprise fusion of the fused pair (cytoplast-donor cell, reconstructed embryo) with at least one cytoplast, such as at least two cytoplasts, for example three cytoplasts, such as four cytoplasts, for example five cytoplasts, such as six cytoplasts, for example seven cytoplasts, such as eight cytoplasts. The second step of fusion with fusion of at least one cytoplast and the fused pair may be performed sequentially or simultaneously. In one embodiment the at least two cytoplasts are fused to the fused pair simultaneously. In another embodiment the at least two cytoplasts are fused to the fused pair sequentially.

In one embodiment of the invention the second step of fusion may also be an activation step wherein the reconstructed embryo is activated to enter mitosis. As described elsewhere herein.

### Activation

In a preferred embodiment the reconstructed embryo may be allowed to rest prior to activation for a period of time in order to allow for the nucleus of the donor cell to reset its genome and gain toti potency in the novel surroundings of the enucleated cytoplast. The reconstructed embryo may for example rest for one hour prior to activation.

Preferably, the reconstructed embryo may be activated in order to induce mitosis. Methods for activation may preferably be selected from the group of consisting of electric pulse, chemically induced shock, increasing intracellular levels of divalent cations and reducing phosphorylation. A combination of methods may be preferred for activation.

In one particular embodiment of the invention the activation and the second step of fusion may be performed simultaneously. However, the activation of the reconstituted embryo and the at least one additional step of fusion between the reconstructed embryo and the at least one cytoplast may be performed sequentially.

Reducing the phosphorylation of cellular proteins in the reconstructed embryo by known methods such as for example by the addition of kinase inhibitors may activate the reconstituted embryo. A preferred embodiment may involve the use of agents that inhibit protein synthesis, for example cycloheximide. A further preferred embodiment may be using agents that inhibit spindle body formation, for example cytochalasin B.

In one embodiment of the invention the intracellular levels of divalent cations may be increased. Divalent cations such as for example calcium may be in comprised in the activation medium. Preferably, the cations may enter the reconstructed embryo, particularly upon subjecting the reconstructed embryo to an electric pulse. In a preferred embodiment the electric pulse may cause entering of calcium into the reconstructed embryo.

The application of an electrical pulse using direct current may be an activation step. However, in a preferred embodiment the electrical pulse applied for activation may also serve as an additional fusion step.

Prior to applying an electrical pulse using direct current the at least one cytoplast and the at least one reconstructed embryo may be aligned by the application of alternating current. The alternating current may be in the range of the range of 0.06 to 0.5 KV/cm, such as 0.1 to 0.4 KV/cm, for example 0.15 to 0.3 KV/cm. In a preferred embodiment alignment of the at least one cytoplast and the donor cell may be performed using alternating current at 0.2 KV/cm.

Activation may be induced by the application of direct current across the fusion plane of the at least one cytoplast and the donor cell. Direct current in the range of 0.2 to 5 KV/cm, such as 0.4 to 5 KV/cm, for example 0.5 to 5 KV/cm. Another preferred embodiment of the present invention is the application of direct current in the range of 0.5 to 2 KV/cm. In a further preferred embodiment the direct current may be 0.7 KV/cm.

The direct current may preferably be applied for in the range of 10 to 200 micro seconds, such as 25 to 150 micro seconds, for example 50 to 100 micro seconds. A particular embodiment may be 80 micro seconds.

In an especially preferred embodiment fusion with direct current may be using a direct current of 0.7 KV/cm for 80 micro seconds.

An especially preferred embodiment of activation according to the present invention may be use of an electrical pulse in combination with subjecting the reconstructed embryo to agents that inhibit protein synthesis, spindle body formation, and divalent cations.

Activation may be performed by any combination of the methods described above.

### In Vitro Culture of Embryos

One aspect of the invention relates to a method of in vitro culturing embryos, whereby the blastocyst rate increased to 25.3%. Thus, a method of culturing a reconstructed embryo is within the scope of the present invention, comprising the steps of a) establishing at least one oocyte having at least a part of zona pellucida, b) separating the oocyte into at least two parts obtaining an oocyte having a nucleus and at least one cytoplast, c) establishing a donor cell or cell nucleus having desired genetic properties, d) fusing at least one cytoplast with the donor cell or membrane surrounded cell nucleus, e) obtaining the reconstructed embryo, f) activating the reconstructed embryo to form an embryo, and e) culturing said embryo.

Another aspect of the invention relates to a method of cell nuclear transfer in which a step of culturing the embryo is included.

In a preferred embodiment in relation to the methods described herein embryos are cultured in vitro in a sequential set of media. Preferably the blastocysts are grown in traditional medium such as for example NCSU37 or equivalent medium as known to a person skilled in the art, wherein glucose is removed and substituted by other agents. One agent may be pyruvate. Another agent may be lactate. The agents may also be combined and replace glucose in the traditional medium.

The embryos may be cultured in the substituted media as described above from Day 0 to Day 3, such as from Day 0 to Day 2.

The pyruvate concentration may range from 0.05 to 1 mM, such as 0.1 to 1 mM, for example 0.125 to 1 mM, such as 0.15 to 1 mM. However the concentration of sodium pyruvate may also range from 0.05 mM to 0.9 mM, such as 0.05 to 0.8 mM, for example 0.05 to 0.7 mM, such as 0.05 to 0.6 mM, for example 0.05 to 0.5 mM, such as 0.05 to 0.4 mM, for example 0.05 to 0.3 mM, such as 0.05 to 0.2 mM. Preferably the concentration ranges between 0.05 to 0.17 mM. A preferred concentration of sodium pyruvate is 0.17 mM.

The lactate concentration may range from 0.5 to 10 mM, such as 0.75 to 10 mM, for example 1 to 10 mM, such as 1.5 to 10 mM, such as 1.75 to 10 mM, for example 2 to 10 mM, such as 2.5 to 10 mM. However the concentration of sodium lactate may also range from 0.5 mM to 9 mM, such as 0.5 to 8 mM, for example 0.5 to 7 mM, such as 0.5 to 6 mM, for example 0.5 to 5 mM, such as 0.5 to 4 mM, for example 0.5 to 03 mM. Preferably the concentration ranges between 1 to 5 mM, such as 2 to 4 mM, for example 2 to 3 mM. A preferred concentration of sodium lactate is 2.73 mM.

After the initial glucose-free incubation medium glucose is again replacing the pyruvate and lactate. The embryos may be cultured in the glucose containing medium from Day 4 to Day 3, preferably from Day 3 to Day 7. The glucose concentration may range from 1 to 10 mM, such as 2 to 10 mM, for example 3 to 10 mM, such as 4 to 10 mM, for example 5 to 10 mM. However, the glucose concentration may also range from 1 to 9 mM, such as 2 to 8 mM, for example 3 to 7 mM, such as 4-6 mM. A preferred concentration of glucose according to the present invention is 5.5 mM of glucose.

### Organ or Tissue Donation

In one embodiment, the animals of the invention may be used as a source for organ or tissue donation for humans or other animals, either animals of the same species or animal of other species. Transfer between species is usually termed xenotransplantation. Entire organs that may be transplanted include the heart, kidney, liver, pancreas or lung. Alternatively, parts of organs, such as specific organ tissues may be transplanted or transferred to humans or other animals. In a yet further embodiment, an individual cell or a population of individual cells from an animal of the invention may be transferred to a human being or another animal for therapeutic purposes.

### Cryopreservation

The term ‘cryopreserving’ as used herein can refer to vitrification of an oocyte, cytoplast, a cell, embryo, or pig of the invention. The temperatures employed for cryopreservation is preferably lower than −80 degree C., and more preferably at temperatures lower than −196 degree C. Oocytes, cells and embryos of the invention can be cryopreserved for an indefinite amount of time. It is known that biological materials can be cryopreserved for more than fifty years.

It is within the scope of the present invention that embryos may be cryopreserved prior to transfer to a host pig when employing methods for producing a genetically engineered or transgenic non-human mammal. Such cryopreservation prior to transfer may be at the blastocyst stage the of embryo development. Vitrification is a form of cryopreservation where living cells are rapidly cooled so that the fluid of the cell does not form into ice. Thus, vitrification relates to the process of cooling where cells or whole tissues are preserved by cooling to low sub-zero temperatures, such as (typically) −80 C or −196 C

In particular the invention relates to the vitrification of an oocyte, however, the invention also relates to the vitrification of embryos, preferably embryos at the blastocyst stage. I one embodiment, the embryo is cultured to blastocyst stage prior to vitrification. Especially pig embryos are covered by the present invention. Also vitrified cytoplasts are covered by the present invention, as are cells.

Yet another aspect of the invention relates to the cryopreservation of a pig embryo derived by a method for cell nuclear transfer as described herein comprising a step of vitrifying a pig embryo. A further aspect of the invention relates to pig embryos obtained, or obtainable by the methods provided herein.

### Mitochondria

Cells of the tissue of the modified non-human mammals and/or non-human embryos obtainable by the present invention may harbour mitochondria of different maternal sources. In a preferred embodiment the non-human mammals and/or non-human embryos may harbour mitochondria from only one maternal source, However, in another preferred embodiment the non-human mammals and/or non-human embryos may harbour mitochondria from at least two maternal sources, such as three maternal sources, for example four maternal sources, such as five maternal sources, for example six maternal sources, such as seven maternal sources, for example eight maternal sources, such as nine maternal sources, for example ten maternal sources. The probability of having a specific number of maternal sources can be calculated based on the observed types of mitochondria.

### Evaluation of Treatment

No cure, currently, exists for patients suffering from breast cancer, mitochondria related protein folding disorders and/or epidermolysis bullosa simplex. The symptoms of Epidermolysis Bullosa are treated by taking care of the blisters and wounds, and reducing the risk of new blister forming as well as the risk of infection in the many wounds that develop. Treatment of the blisters and wound can be very time consuming and interfere with the patients normal life, such as the ability to attend school or go to work. Thus, a need exists for efficient animal models, which displays aspects that resemble human breast cancer, mitochondria related protein folding disorders and/or epidermolysis bullosa simplex.

The present invention offers a method for screening the efficacy of a pharmaceutical composition, wherein the method comprises the steps of i) providing the pig model of the present invention, ii) expressing in said pig model the genetic determinant and exerting said phenotype for said disease, iii) administering to the pig model a pharmaceutical composition the efficacy of which is to be evaluated, and iv) evaluating the effect, if any, of the pharmaceutical composition on the phenotype exerted by the genetic determinant when expressed in the pig model.

Furthermore, within the scope of the present invention is a method for evaluating the response and/or the effect of a therapeutical treatment of breast cancer, mitochondria related protein folding disorders and/or epidermolysis bullosa simplex, wherein the method comprises the steps of i) providing the pig model of the present invention, ii) treating said pig model with a pharmaceutical composition exerting an effect on said phenotype, and iii) evaluating the effect observed. Based on the evaluation one could further advise on the treatment based on the observed effects.

In addition, the present invention relates to a method for treatment of a human being suffering from breast cancer, mitochondria related protein folding disorders and/or epidermolysis bullosa simplex, wherein the method comprises the initial steps of i) providing the pig model of the present invention, ii) expressing in said pig model said genetic determinant and exerting said phenotype for said disease, iii) administering to said pig model a pharmaceutical composition the efficacy of which is to be evaluated, and v) evaluating the effect observed, and v) treating said human being suffering from breast cancer, mitochondria related protein folding disorders and/or epidermolysis bullosa simplex based on the effects observed in the pig model.

It is therefore appreciated that the pig model according to the present invention may also receive medicaments for diseases other than breast cancer, mitochondria related protein folding disorders and/or epidermolysis bullosa simplex in order to test the combined effect of a drug for breast cancer, mitochondria related protein folding disorders and/or epidermolysis bullosa simplex and other drugs administered to the pig.

### EXAMPLES

### Breast Cancer

### 1. Construction of a Porcine Model of Breast Cancer

Three approaches have been undertaken in order to introduce the desired constructs which have been used in homologous recombination in porcine fibroblasts which have subsequently been used in nuclear transfer according to the invention to produce genetically modified pigs having a breast cancer phenotype. The three approaches are as follows:

1) introduce the codon 61 BRCA1 mutation (thoroughly studied in hereditary breast cancer patients) in pig somatic cells by knock-in strategy (homologous recombination of a construct containing the codon 61 mutation and a selection gene into the endogenous BRCA1 gene), or 2) to knock out one allele of the BRCA1 gene (homologous recombination of a construct containing a selection gene inside exon 11 sequence of BRCA1 gene into the endogenous BRCA1 gene), or 3) to knock out one allele of the BRCA2 gene (homologous recombination of a construct containing a selection gene inside exon 11 sequence of BRCA2 gene into the endogenous BRCA2 gene).
1) Porcine BRCA1 exon 3 nucleotide substitution T>G resulting in amino acid substitution Cys>Gly (codon 61):

tttngtatgctgaaacttctcaaccagaagaaagggccttcacagT > Ggtcctttgtgtaagaatgatataaccaaaagg

2) Porcine BRCA1 exon 11 area deleted:

```
    1 agcatgagac cagcagttta ttactcacta aagacagaat gaatgtagaa aaggctgaat
  61 tttgtaataa aagcaagcag cctgtcttag caaagagcca acagagcaga tgggctgaaa
121 gtaagggcac atgtaatgat aggcagactc ctaacacaga gaaaaaggta gttctgaata
181 ctgatctcct gtatgggaga aacgaactga ataagcagaa acctgcgtgc tctgacagtc
241 ctagagattc ccaagatgtt ccttggataa cattgaatag tagcatacag aaagttaatg
301 agtggttttc tagaagcgat gaaatgttaa cttctgacga ctcacaggac aggaggtctg
361 aatcaaatac tggggtagct ggtgcagcag aggttccaaa tgaagcagat ggacatttgg
421 gttcttcaga gaaaatagac ttaatggcca gtgaccctca tggtgcttta atacgtgaac
481 gtgaaagagg gcactccaaa ccagcagaga gtaatattga agataaaata tttgggaaaa
541 cctatcggag gaaggcaagc ctccctaact tgagccacgt aattgaagat ctaattttag
601 gagcatctgc tgtagagcct caaataacac aagagcgccc cctcacaaat aaactaaagc
661 ggaaaaggag aggtacatc
```

3) Porcine BRCA2 exon 11. Area deleted within this sequence:

```
      1 ggtccaggat gtttctcttc aagcaaatgt aatgattctg atgtttcaat atttaaggta
    61 gaaaattata gcagtgataa aagtttaagt gagaaataca ataaatgcca actgatacta
  121 aaaaataaca ttgaaaggac tgctgacatt tttgttgaag aaaatactga cggttacaag
  181 agaaatactg aaaataaaga caacaaatgt actggtcttg ctagtaactt aggaggaagc
  241 tggatggaca gtgcttcaag taaaactgat acagtttata tgcacgaaga tgaaactggt
  301 ttgccattta ttgatcacaa catacatcta aaattaccta accactttat gaagaaggga
  361 aatactcaaa ttaaagaagg tttgtcagat ttgacttgtt tggaagttat gagagccgaa
  421 gaaacatttc atattaatac atcaaataaa cagtcaactg ttaataagag gagccaaaaa
  481 ataaaagatt ttgatgtttt tgatttgtcc tttcagagtg caagtgggaa aaacatcaga
  541 gtctctaaag agtcattaaa taaagctgta aatttctttg acgaaaaatg cacagaagaa
  601 gaattgaata acttttcaga ttcctcaaat tctgaaatac ttcctggcat aaatatcaac
  661 aaaataaaca tttcaagcca taaggaaaca gattcggaca aaaacaaact attgaaagaa
  721 agtgacccag ttggtattga aaatcaatta ctgactctcc agcaaagatc agaatgtgaa
  781 atcaaaaaga tcgaagaacc taccatgctg ggttttcata cagctagtgg gaaaaaagta
  841 aaaattgcga aggaatcgtt ggacaaagtg aaaaatcttt ttgatgaaac aaagcaagat
  901 agtagtgaaa ccactaattc tagccatcaa ggggtaaaaa cacagaagga cagagaggta
  961 tgtaaagaag agcttgaatt aacattcgag acagttgaaa taactgcctc aaagcatgaa
1021 gaaatacgga attttttaga ggagaaaaaa cttgtttcta aggagatcac catgccaccc
1081 aggctcttac gtcatcattt acacagacaa actgaaaatc tcagcatgtc aaacagtatc
1141 cccctaaaag gtaaagtaca tgaaaatatg gaagaagaaa catcttgtca cacagatcag
1201 tccacttgtt cagccattga aaattcagca ttaacatttt acacaggaca tggcagaaaa
1261 atttctgtga atcaggcttc cgtatttgaa gccaaaaagt ggcttagaga aggagaattg
1321 gacgatcaac cagaaaacgt agattctgcc aaggtcatat gtttaaagga atatgctagg
1381 gattatgtag gaaatccttt gtgtgggagt agttcaaaca gtatcataac tgaaaatgac
1441 aaaaatctcc ctgaaaaaca aaattcaact tatttaagta acagtgtgtc taacaactat
1501 tcataccatt ctgatttttg tcattccaat gaggtgctca gcaaatcaga atctctctca
1561 gaaaataaaa ttggtaattc tgatactgag ccagcagtga agaatgtcaa agacagaaaa
1621 gacacttgtt tttctgaaga gatatccacc gtaagagaag caaacacaca cccacaagct
1681 gtagatgaag acagctgggt tcggaagctt gtgattaact ctacaccatg caaaaataaa
1741 aatacacctg gtgaagtgtc caatctaatt caaataattt tgagatagag ccacctgcat
1801 tcagtacaag tgggaacata gcctttgttt cacatgaaac agacgtgaga gagaggtttg
1861 cagacaacaa caggaaggcg attaagcaaa acactgagag tatgtcaggc tcttgccaaa
1921 tgaaaattat gactggcgct cataaggcat tgggtgattc agaggatgtt attttcccta
1981 actctccaga tagtgaagaa catattacac gttcacagga ggtttttcct gaaattcaaa
2041 gtgaacaaat tttacaacat gacccaagtg tatccggatt ggagaaagtt tctgaaatgc
2101 caccttgtca tattaactta aaaacttttg atatacataa gtttgatatg aaaagacatc
2161 ccatgtcagt ctcttctatg aatgattgtg gggtttttag cacagcaagt ggaaaatctg
2221 tacaagtatc agatactgca ttacaaaaag cgagacaagt attttctaag acagaagatg
2281 tggctaagcc attcttttcc agagcagtta aaagtgatga agaacattca gacaagtaca
2341 caagagaaga aaatgctatg atgcatcccc ccccaaattt cctgtcatct gctttctccg
2401 gatttagtac agcaagtgga aaacaggttc cagtttctga gagtgcctta tgcaaagtga
2461 agggaatgtt tgaggaattt gatttaatgg gaactgaatg tagacttcag cattcaccta
2521 catctagaca agatgtgtca aagatacttc ctctctccga gattgatgag agaaccccag
2581 aacactctgt aagttcccaa acagagaaag cctacaatga acaatttaaa ttaccagata
2641 gctgtaacac tgaaagcagt tcttcagaaa ataatcactc tgttaaagtt tctcccgatc
2701 tctctcggtt taagcaagac aaacagttgg tatcaggagc aaaagtatca cttgttgaga
2761 acattcatcc atcgggaaaa gaa
```

### Mitochondria Related Protein Folding Disorders

### 1. Cloning of Constructs

Genetically modified pigs have been generated with a naturally occurring mutated gene for Ornithine TransCarbamylase (OTC) from rat which lacks the carbamyl phosphate-binding domain. The defective protein enters the mitochondria but cannot fold properly. Accumulation of misfolded proteins is the hallmark of a multitude of degenerative processes including neurodegenerative diseases, such as Alzheimers disease, Parkinsons disease, and Huntingtons Chorea. It is generally believed that the accumulation of misfolded protein—through creation of cellular stress—is linked to the observed mitochondrial dysfunction and neuronal cell death. However, the relationship between the protein misfolding, which often occur outside the mitochondria, and the mitochondrial dysfunction remains unclear. We are in the process of generating genetically modified pigs with a naturally occurring mutated gene for Ornithine TransCarbamylase (OTC) which lacks the carbamyl phosphate-binding domain. The defective protein enters the mitochondria but cannot fold properly.

Rat Otc-Δ cDNA (deleted area in grey): The sequence is cloned into pN1-EGFP (Clonteq) with a CAGGS promoter and as a fusiogene with EGFP (CAGGS-OTCΔ-EGFP and transfected into porcine fetal fibroblasts:

atggttcgaaattttcggtatgggaagccagtccagagtcaagtacagct
gaaaggccgtgacctcctcaccctgaagaacttcacaggagaggagattc
agtacatgctatggctctctgcagatctgaaattcaggatcaaacagaaa
ggagaatacttgcctttattgcaagggaaatccttagggatgatttttga
gaaaagaagtactcgaacaagactgtccacagaaacaggcttcgctcttc
tgggaggacatccttcttttcttaccacacaagacattcacttgggcgtg
aatgaaagtctcacagacacagctcgtgtgttatctagcatgacagatgc
agtgttagctcgagtgtataaacaatcagatctggacatcctggctaagg
aagcaaccatcccaattgtcaacggactgtcagacctgtatcatcctatc
cagatcctggctgattaccttacactccaggaacactatggctctctcaa
aggtctcaccctcagctggataggagatgggaacaatatcctgcactcca
tcatgatgagtgctgcaaaattcgggatgcaccttcaagcagctactcca
aagggttatgagccagatcctaatatagtcaagctagcagagcagtatgc
caaggagaatggtaccaggttgtcaatgacaaatgatccactggaagcag
cacgtggaggcaatgtattaattacagatacttggataagcatgggacaa
gaggatgagaagaaaaagcgtcttcaagctttccaaggttaccaggttac
aatgaagactgctaaagtggctgcgtctgactggacgtttttacactgct
tgcctagaaagccagaagaagtagatgatgaagtgttttattctccgcgg
tcattagtgttcccagaggcagaaaatagaaagtggacaatcatggctgt
catggtatccctgctgacagactactcacctgtgctccagaagccaaagt
tctgatgcctgcaagaggacgaaaaacccaaaagacaaaaaaatctgttc
tttagcagcagaataagtcagtttatgtagaaaagagaagaattgaaatt
gtaaacacatccctagtgcgtgatataattatgtaattgctttgctattg
tgagaattgcttaaagcttttagtttaagtgctgggcattttattatcct
gcttgacttgacttaagcactctcttcaattcacaacttctgaatgatat
ttgggtttcatattaattatcatacacatttccttccactaagcattaaa
cactatgcttacaatgcataccatctaagtcattaaatgtaatccatgct
tattacctt

### Epidermolysis Bullosa Simplex

### 1. Construction of a Porcine Model of Epidermolysis Bullosa Simplex

One example of a transgene that could be used to produce a transgenic non-human mammal as a disease model for epidermolysis bullosa simplex is the human keratin 14 gene, comprising a mutation as shown below in bold.

The sequence of the transgene integrated in porcine fetal fibroblasts (donor cell) comprises the human keratin 14 promoter and keratin 14 cDNA including start and stop codons (in bold) and the disease causing mutation (in bold and underlined) as described by Sørensen et al., J Invest Dermatol. 1999 February; 112(2):184-90). The fragment is cloned into pN1-EGFP (clontech) containing polyA signal for gene expression and a Neomycin selection gene for selection of cell clones with the transgene integrated.

```
      1 aagcttatat tccatgctag ggttctggtg ttggtgcgtg gggttggggt gggactgcag
    61 aagtgccttt taagattatg tgattgactg atctgtcatt ggttccctgc catctttatc
  121 ttttggattc ccctcggagg aggggaggaa ggagtttctt ttgggtttta ttgaatcaaa
  181 tgaaagggaa agtagaggtg ttcctatgga ggggaggaag gagtttcttt tgggttttat
  241 tgaatcaaat gaaagggaaa gtagaggtgt tcctatgtcc cgggctccgg agcttctatt
  301 cctgggccct gcataagaag gagacatggt ggtggtggtg gtgggtgggg gtggtggggc
  361 acagaggaag ccgatgctgg gctctgcacc ccattcccgc tcccagatcc ctctggatat
  421 agcaccccct ccagtgagca cagcctcccc ttgccccaca gccaacagca acatgcctcc
  481 caacaaagca tctgtccctc agccaaaacc cctgttgcct ctctctgggg aaattgtagg
  541 gctgggccag ggtgggggga ccattctctg cagggagatt aggagtgtct gtcaggggcg
  601 ggtggagcgg ggtggggccc tggcttactc acatccttga gagtcctttg ctggcagatt
  661 tggggagccc acagctcaga tgtctgtctc agcattgtct tccaagctcc taggccacag
  721 tagtggggcg ctcccttctc tggcttcttc tttggtgaca gtcaaggtgg ggttgggggt
  781 gacgaagggt cctgcttctc ttctaggagc agttgatccc aggaagagca ttggagcctc
  841 cagcaggggc tgttggggcc tgtctgagga gataggatgc gtcaggcagc cccagacacg
  901 atcacattcc tctcaacatg cctgccgggg tctgtggagc cgaggggctg atgggagggt
  961 ggggtggggg ccggaagggt ttgctttggg aggttgtctg ggagattgct gaagttttga
1021 tatacacacc tccaaagcag gaccaagtgg actcctagaa atgtcccctg acccttgggg
1081 cttcaggagt cagggaccct cgtgtccacc tcagccttgc ccttgcacag cccagctcca
1141 ctccagcctc tactcctccc cagaacatct cctgggccag ttccacaagg ggctcaaacg
1201 agggcacctg agctgcccac actagggatg ttctgggggt ctgagaagat atctggggct
1261 ggaagaataa aaggcccccc taggcctgtt cctggatgca gctccagcca ctttggggct
1321 aagcctgggc aataacaatg ccaacgaggc ttcttgccat actcggttta caaaaccctt
1381 tacatacatt gtcgcattgg attctcagag ctgactgcac taagcagaat agatggtatg
1441 actcccactt tgcagatgag aacactgagg ctcagagaag tgcgaagccc tgggtcacag
1501 aggcgtaaat gcagagccag gacccacctg aagacccacc tgactccagg atgtttcctg
1561 cctccatgag gccacctgcc ctatggtgtg gtggatgtga gatcctcacc atagggagga
1621 gattagggtc tgtgctcagg gctggggaga ggtgcctgga tttctctttg atggggatgt
1681 tggggtggga atcacgatac acctgatcag ctgggtgtat ttcagggatg gggcagactt
1741 ctcagcacag cacggcaggt caggcctggg agggcccccc agacctcctt gtctctaata
1801 gagggtcatg gtgagggagg cctgtctgtg cccaaggtga ccttgccatg ccggtgcttt
1861 ccagccgggt atccatcccc tgcagcagca ggcttcctct acgtggatgt taaaggccca
1921 ttcagttcat ggagagctag caggaaacta ggtttaaggt gcagaggccc tgctctctgt
1981 caccctggct aagcccagtg cgtgggttcc tgagggctgg gactcccagg gtccgatggg
2041 aaagtgtagc ctgcaggccc acacctcccc ctgtgaatca cgcctggcgg gacaagaaag
2101 cccaaaacac tccaaacaat gagtttccag taaaatatga cagacatgat gaggcggatg
2161 agaggaggga cctgcctggg agttggcgct agcctgtggg tgatgaaagc caaggggaat
2221 ggaaagtgcc agacccgccc cctacccatg agtataaagc actcgcatcc ctttgcaatt
2281 tacccgagca ccttctcttc actcagcctt ctgctcgctc gctcacctcc ctcctctgca
2341 ccatgactac ctgcagccgc cagttcacct cctccagctc catgaagggc tcctgcggca
2401 tcgggggcgg catcgggggc ggctccagcc gcatctcctc cgtcctggcc ggagggtcct
2461 gccgcgcccc cagcacctac gggggcggcc tgtctgtctc atcctcccgc ttctcctctg
2521 ggggagccta cgggctgggg ggcggctatg gcggtggctt cagcagcagc agcagcagct
2581 ttggtagtgg ctttggggga ggatatggtg gtggccttgg tgctggcttg ggtggtggct
2641 ttggtggtgg ctttgctggt ggtgatgggc ttctggtggg cagtgagaag gtgaccatgc
2701 agaacctcaG tgaccgcctg gcctcctacc tggacaaggt gcgtgctctg gaggaggcca
2761 acgccgacct ggaagtgaag atccgtgact ggtaccagag gcagcggcct gctgagatca
2821 aagactacag tccctacttc aagaccattg aggacctgag gaacaagatt ctcacagcca
2881 cagtggacaa tgccaatgtc cttctgcaga ttgacaatgc ccgtctggcc gcggatgact
2941 tccgcaccaa gtatgagaca gagttgaacc tgcgcatgag tgtggaagcc gacatcaatg
3001 gcctgcgcag ggtgctggac gaactgaccc tggccagagc tgacctggag atgcagattg
3061 agagcctgaa ggaggagctg gcctacctga agaagaacca cgaggaggag atgaatgccc
3121 tgagaggcca ggtgggtgga gatgtcaatg tggagatgga cgctgcacct ggcgtggacc
3181 tgagccgcat tctgaacgag atgcgtgacc agtatgagaa gatggcagag aagaaccgca
3241 aggatgccga ggaatggttc ttcaccaaga cagaggagct gaaccgcgag gtggccacca
3301 acagcgagct ggtgcagagc ggcaagagcg agatctcgga gctccggcgc accatgcaga
3361 acctggagat tgagctgcag tcccagctca gcatgaaagc atccctggag aacagcctgg
3421 aggagaccaa aggtcgctac tgcatgcagc tggcccagat ccaggagatg attggcagcg
3481 tggaggagca gctggcccag ctccgctgcg agatggagca gcagaaccag gagtacaaga
3541 tcctgctgga cgtgaagacg cggctggagc aggagatcgc cacctaccgc cgcctgctgg
3601 agggcgagga cgcccacctc tcctcctccc agttctcctc tggatcgcag tcatccagag
3661 atgtgacctc ctccagccgc caaatccgca ccaaggtcat ggatgtgcac gatggcaagg
3721 tggtgtccac ccacgagcag gtccttcgca ccaagaacga ctacaaggac gacgatgaca
3781 agtg aggatcc
```

### Common

### 3. Handmade Cloning (HMC) and Establishment of Pregnancies

For the cloning and delivery of transgenic piglets, transgenic donor cells carrying the constructs as described in examples relating to breast cancer, epidermolysis bullosa simplex and mitochondria related protein folding disorders, transgenic donor cells are used in HMC.

Except where otherwise indicated all chemicals were obtained from Sigma Chemical Co. (St Louis, Mo., USA).

### Oocyte Collection and In Vitro Maturation (IVM)

Cumulus-oocyte complexes (COCs) are aspirated from 2 to 6 mm follicles from slaughterhouse-derived sow ovaries and matured in groups of 50 in 400 μl IVM medium consisting of bicarbonate-buffered TCM-199 (GIBCO BRL) supplemented with 10% (v/v) cattle serum (CS), 10% (v/v) pig follicular fluid, 10 IU/ml eCG, 5 IU/ml hCG (Suigonan Vet; Skovlunde, Denmark) at 38.5° C. in 5% CO₂ in humidified air in the Submarine Incubation System (SIS; Vajta et al., 1997) for 41-44 h.

HMC is performed by a procedure based on partial digestion of the zona pellucida, as described earlier (Du et al., 2005 and 2007). Matured COCs was freed from cumulum cells in 1 mg/ml hyaluronidase in Hepes-buffered TCM-199. From this point (except where otherwise indicated) all manipulations are performed on a heated stage adjusted to 39° C., and all drops used for handling oocytes were of 20 μl covered with mineral oil. Zonae pellucidae of are partially digested with 3.3 mg/ml pronase solution dissolved in T33 (T for Hepes-buffered TCM 199 medium; the number means percentage (v:v) of CS supplement, here 33%) for 20 s, then oocytes are washed quickly in T2 and T20 drops. Oocytes with distended and softened zonae pellucidae are lined up in T20 drops supplemented with 2.5 μg/ml cytochalasin B. With a finely drawn glass pipette, oocytes are rotated to locate the polar body on the surface. By oriented bisection with an Ultra Sharp Splitting Blade (AB Technology, Pullman, Wash., USA) less than half of the cytoplasm close to the polar body is removed manually from the remaining putative cytoplast.

Transgenic donor fibroblasts grown to a confluent monolayer in DMEM supplemented with 10% FCS are trypsinized and kept in T20 (Kragh et al., 2004). Fusion is performed in two steps. For the first step, 50% of the available cytoplasts were transferred into 1 mg/ml of phytohemagglutinin (PHA; ICN Pharmaceuticals, Australia) dissolved in TO for 3 s, then each one is quickly dropped over a single APPsw transgenic fibroblast. After attachment, cytoplast-fibroblast cell pairs are equilibrated in fusion medium (0.3 M mannitol and 0.01% PVA) for 10 s and transferred to the fusion chamber (BTX microslide 0.5 mm fusion chamber, model 450; BTX, SanDiego, Calif., USA). Using an alternating current (AC) of 0.6 kV/cm and 700 kHz, pairs are aligned to the wire of a fusion chamber with the somatic cells farthest from the wire, then is fused with a direct current of 2.0 kV/cm for 9 μs. After the electrical pulse, cell pairs are incubated in T10 drops to observe whether fusion has occurred.

Approximately 1 h after the first fusion, each pair is fused with another cytoplast and activated simultaneously in activation medium (0.3 M mannitol, 0.1 mM MgSO₄, 0.1 mM CaCl₂ and 0.01% PVA). By using an AC of 0.6 kV/cm and 700 kHz, one fused pair and one cytoplast is aligned to one wire of the fusion chamber, with fused pairs contacting the wire, followed by a single DC pulse of 0.85 kV/cm for 80 μs. When fusion is observed in T10 drops, reconstructed embryos are transferred into porcine zygote medium 3 (PZM-3; Yoshioka et al., 2002) supplemented with 5 μg/ml cytochalasin B and 10 μg/ml cycloheximide. After a 4 h incubation at 38.5° C. in 5% CO₂, 5% O₂ and 90% N₂ with maximum humidity, embryos are washed three times in PZM-3 medium before culture

### Embryo Culture and Transfer

Embryos are cultured at 38.5° C. in 5% CO₂, 5% O₂ and 90% N₂ with maximum humidity in PZM-3 medium in the well of well system (WOWs; Vajta et al., 2000). Day 5 and 6 blastocysts with clearly visible inner cell mass are surgically transferred to Danish landrace sows on day 4 or 5 after weaning. Pregnancies are diagnosed by ultrasonography on day 21 and confirmed every second week. Piglets are delivered by Caesarean section on day 114, 24 h after treatment with prostaglandin F2.

Steps 2. to 3. are applicable for breast cancer, mitochondria related protein folding disorders and/or epidermolysis bullosa simplex

### 2. Establishing a Transgenic Porcine Fibroblast Cell

Based on the well-described mechanisms of SB transposition (4-8) and Flp recombination (9, 10), the present invention discloses a new target vector for site-specific integration into the genome. This vector carries within the context of a SB transposon vector a bicistronic gene cassette containing (i) the FRT recombination site embedded in the coding sequence of eGFP and (ii) an IRES-driven puromycin resistance gene. We demonstrate efficient selective plasmid insertion into SB-tagged genomic loci. In an attempt to further improve the performance of these vectors, we have analyzed the effect of insulator elements, believed to protect inserted foreign genes against transcriptional silencing, within the context of SB vectors. Our investigations indicate that insulators flanking the FRT gene expression cassette may serve to maintain and stabilize gene expression of Flp-inserted transgenes.

Two nonviral integration technologies are employed in the present invention, the SB transposon system and the Flp recombinase, in a combined effort to achieve active locus detection, mediated by SB, and site-directed insertion at an attractive site, mediated by Flp. A bi-phased technology is disclosed in which an integrating SB vector, carrying a reporter gene and a selective marker gene, may first serve as a reporter for continuous gene expression and hence as a target for gene insertion (FIG. 19). By using an actively integrated vector as opposed to plasmid DNA that is randomly recombined into the genome we certify (i) that only a single copy, and not concatemers, of the vector are inserted and, moreover, (ii) that the reporter cassette is not flanked by sequences derived from the bacterial plasmid backbone which may have a detrimental effect on the locus activity over time. In a second modification step this vector may serve as a target for insertion of one or more gene expression cassettes in a well-characterized locus.

### Vector Construction

The SB transposon-based vector used in this study was derived from the pSBT/SV40-GFIP.loxP vector. This vector contains, within the context of a SB transposon, a bicistronic FRTeGFP-IRES-puro (GFIP) cassette flanked upstream by an ATG start codon and downstream by a poly A sequence. Moreover, the vector contains a recognition site for the Cre recombinase (loxP) located between the upper inverted repeat of the vector and the SV40 promoter driving expression of the FRTeGFP-IRES-puro cassette.

Construction of pSBT/SV40-GFIP.loxP Vector

The pSBT/RSV-GFIP vector contains the terminal inverted of the SB DNA transposon flanking a FRT-GFP.IRES.puro bicistronic gene cassette driven by a promotor derived from Rous sarcoma virus (RSV). The eGFP sequence was amplified from peGFP.N1 (Clontech) using a forward primer containing the 48-bp FRT sequence. To analyze FRT-GFP functionality, the FRT-eGFP fusion was inserted into an expression vector containing the SV40 promoter. The PCR-fragment containing FRT-tagged eGFP fusion gene was digested with MluI and XmaI and inserted into MluI/XmaI-digested pSBT/RSV-hAAT (pT/hAAT in ref. (8), obtained from Mark Kay, Stanford University, USA), generating a transposon vector with RSV-driven eGFP expression (pSBT/RSV-eGFP). An IRES-puro cassette was PCR-amplified from pecoenv-IRES-puro (provided by Finn Skou Pedersen, University of Aarhus, Denmark), digested with XmaI, and inserted into XmaI-digested pSBT/RSV-eGFP, generating pSBT/RSV-GFIP (see FIG. 20). Alternative versions of this vector containing the SV40 promoter (pSBT/SV40-GFIP) and the promoter derived from the human ubiquitin gene (pSBT/Ubi-GFIP), were generated. In addition, by inserting a Cre recombination target site (loxP) into the MluI site located between the left inverted repeat of the transposon and the SV40 promoter of pSBT/SV40-GFIP, the vector pSBT/SV40-GFIP.loxP was created. The donor plasmid pcDNA5/FRT, containing a FRT-hygro fusion gene without a start codon, was obtained from Invitrogen. The Flp-encoding plasmid, pCMV-Flp was obtained from A. Francis Stewart, University of California San Francisco, USA). This plasmid encodes the enhanced Flp variant designated Flpx9 (11). A SB-vector containing two copies of the 1.2-kb chicken DNase hypersensitive site 4 (cHS4)-derived insulator element (12, 13) was generated by inserting PCR-amplified cHS4 sequences and an intervening linker into NotI/SpeI-digested pSBT/PGK-puro (obtained from Mark Kay, Stanford University, USA). The PGK-puro cassette was cloned back into construct by using restriction sites located in the linker, generating pSBT/cHS4.PGK-puro.cHS4

For further use in pigs an alternative Cre recognition site (loxP-257) was inserted into a unique AscI site that was created by mutagenesis at a position located between the poly A sequence and the lower inverted repeat of the vector. This vector was designated pSBT/loxP.SV40-GFIP.loxP257. The presence of two Cre recombination sites allows Cre recombinase-mediated cassette exchange after Flp-based plasmid insertion, thereby facilitating, if needed, removal of plasmid sequences and selection genes.

### SB Transposition in Primary Pig Fibroblasts

The SB transposon vectors, either SBT/PGK-puro or the target transposon SBT/loxP.RSV-GFIP.loxP257, were inserted into the genome of pig fibroblast by co-transfecting (using Fugene-6 from Roche) 1.5 μg pSBT/lox.RSV-GFIP.loxP257 (or pSBT/PGK-puro) with 1.5 μg pCMV-SB (or 1.5 μg pCMV-mSB as a negative control). pCMV-SB (rights held by Perry Hackett, University of Minnesota, Minnesota, USA) encodes the Sleeping Beauty transposase reconstructed from fossil DNA transposable elements of salmoid fish. pCMV-SB, pCMV-mSB, and the hyperactive variant pCMV-HSB3 were obtained from Mark Kay, Stanford University, USA. SB-tagged cell clones appeared as a result of selecting transfected cells with puromycin (0.5 μg/ml). Colonies were fixed and stained in methylene blue in methanol and subsequently counted.

### Solid SB Transposition in Primary Pig Fibroblasts

SB transposes efficiently in most mammal cells but with higher efficacy in human cells than in murine cells. Transposition of SB vectors has never been analyzed in porcine cells, and we therefore initially tested activity in primary pig fibroblasts. We utilized a standard transposon encoding a puromycin resistance gene (SBT/PGK-puro) and found decent levels of transposition, resulting in about 75 drug-resistant colonies in cultures of fibroblasts co-transfected with pSBT/PGK-puro and pCMV-SB (FIG. 21). Less than 3 colonies appeared after transfection with pSBT/PGK-puro and pCMV-mSB, the latter which encodes an inactive version of the transposase. Interestingly, a mean of almost 140 colonies was obtained using the hyperactive transposase variant HSB3, indicating that HSB3 also in porcine cells mediates higher levels of transposition compared to the original SB transposase.

### Efficient Insertion of a FRT-Tagged SB Vector in Pig Fibroblasts

To generate SB-tagged cell clones containing a Flp recombination target site for site-specific gene insertion, we co-transfected the pSBT/loxP.SV40-lopP257 plasmid with pCMV-mSB, pCMV-SB, and pCMV-HSB3, respectively. HSB3 again showed the highest activity, resulting in about 30 drug-resistant colonies after transfection of 3H 10⁴ fibroblasts (FIG. 22).

Puromycin-resistant colonies were isolated and expanded. Clone analysis by fluorescence microscopy demonstrated efficient FRTeGFP expression (FIG. 23), demonstrating vector functionality and easy FRTeGFP detection in pig fibroblasts. These fluorescent cell clones carrying the Flp FRT recombination sequence are currently being used for creation of cloned transgenic animals by hand-made cloning.

Verification of SBT/loxP.SV40-GFIP.loxP257 as target for Flp recombination Due to limitations of long-term growth of primary pig fibroblasts in tissue culture we were not able to demonstrate Flp-based gene insertion into FRT-tagged SB vectors in pig fibroblasts. We therefore chose to test functionality of the FRT-containing vector by a standard set of recombination experiments carried out in HEK-293 cells. We generated clones of HEK-293 cells containing the transposed SBT/loxP.SV40-GFIP.loxP257 vector. By co-transfection of such clones with (i) a pcDNA5/FRT-derived substrate plasmid containing a FRT-hygro fusion gene and a red fluorescent protein (RFP) expression cassette and (ii) a plasmid encoding the Flp recombinase (pCMV-Flpx9), we subsequently identified hygromycin B resistant colonies. By fluorescence microscopy we observed that site-specifically engineered clones, as expected, turned-off eGFP expression and turned-on RFP expression (data not shown). This ‘green-to-red’ phenotypic change indicates that the integrated SB-derived target vector serves as acceptor site for Flp-based recombination.

In conclusion, the Sleeping Beauty DNA transposon-based vector of the present invention serves in its integrated form as a target for recombinase-based gene insertion. The SB vector is efficiently transferred by cut-and-paste transposition into the genome of primary porcine fibroblasts and therefore is not flanked by plasmid-derived bacterial sequences. Use of these genetically engineered primary cells in for example microinjection and hand-made cloning allows subsequent detailed analyses of SB vector-derived eGFP expression in cloned pigs and identification of animals with attractive expression profiles (e.g. ubiquitous, tissue-specific). Primary fibroblasts from such ‘master pigs’ is further modified by Flp-based recombination, allowing site-directed gene insertion in a SB vector-tagged locus which is not silenced in the tissue of interest. Cloned pigs harboring a site-specifically inserted disease gene of interest or a shRNA expression cassette for downregulation of endogenous genes can be generated by a second round of animal cloning.

### 3. Production of Disease Model by Handmade Cloning

Except where otherwise indicated all chemicals were obtained from Sigma Chemical Co. (St Louis, Mo., USA).

### Oocyte Collection and In Vitro Maturation (IVM)

Cumulus-oocyte complexes (COCs) were aspirated from 2-6 mm follicles from slaughterhouse-derived sow or gilt ovaries. COCs were matured in groups of 50 in 400 μl bicarbonate-buffered TCM-199 (GIBCO BRL) supplemented with 10% (v/v) cattle serum (CS), 10% (v/v) pig follicular fluid, 10 IU/ml eCG, 5 IU/ml hCG (Suigonan Vet; Skovlunde, Denmark) at 38.5° C. in the “Submarine Incubation System” (SIS; Vajta, et al. 1997) in 5% CO₂ in humidified air for 41-44 hours.

### In Vitro Fertilization (IVF)

IVF experiments were performed with in vitro matured oocytes in 3 identical replicates. After maturation, COCs were washed twice with mTBM containing 2 mM caffeine (mTBMₜₑᵣₜ) and transferred in groups of 50 to 400 μl mTBMₜₑᵣₜ. Freshly ejaculated semen was treated as described previously (Booth, et al., in press). After 2 h capacitation at 38.5° C. and in 5% CO₂ in humidified air, sperm was added to the oocytes with the adjusted final concentration of 1×10⁵ sperm/ml. Fertilization was performed at 38.5° C. and in 5% CO₂ in humidified air in the SIS for 3 h. After the insemination, the presumptive zygotes were vortexed in mTBM_{fe}, to remove cumulus cells before washing in IVC medium and placing in culture dishes (see Embryo culture and evaluation).

### Handmade Cloning (HMC)

The applied HMC method was based on our previous work in cattle and pig (Kragh, et al., 2004; Peura and Vajta, 2003; Vajta, et al., 2003), but with significant modifications. Briefly, at 41 h after the start of maturation, the cumulus investment of the COCs was removed by repeated pipetting in 1 mg/ml hyaluronidase in Hepes-buffered TCM199. From this point (except where otherwise indicated), all manipulations were performed on a heated stage adjusted to 39° C., and all drops used for handling oocytes were of 20 μl volume covered with mineral oil. Oocytes were briefly incubated in 3.3 mg/ml pronase dissolved in T33 (T for Hepes-buffered TCM 199 medium; the number means percentage (v/v) of CS supplement, here 33%) for 5 s. Before the oocytes started to become misshaped in pronase solution, they were picked out and washed quickly in T2 and T20 drops. Oocytes with partially digested but still visible zona were lined up in drops of T2 supplemented with 3 mg/ml polyvinyl alcohol (TPVA) and 2.5 μg/ml cytochalasin B. Trisection instead of bisection was performed manually under stereomicroscopic control with Ultra Sharp Splitting Blades (AB Technology, Pullman, Wash., USA; FIG. 24a). Fragments of trisected oocytes were collected and stained with 5 μg/ml Hoechst 33342 fluorochrome in TPVA drops for 5 min, then placed into 1 μl drops of the TPVA medium on the bottom of a 60 mm Falcon Petri dish covered with oil (3-4 fragments per drop). Using an inverted microscope and UV light, positions of fragments without chromatin staining (cytoplasts) were registered and later collected under a stereomicroscope in T10 drops until the start of the fusion.

Fetal fibroblast cells were prepared as described previously (Kragh, et al., in press). Fusion was performed in two steps where the second one included the initiation of activation, as well. For the first step, one third of the selected cytoplasts (preferably the smaller parts) were used. With a finely drawn and fire-polished glass pipette, 10 cytoplasts were transferred as a group to 1 mg/ml of phytohaemagglutinin (PHA; ICN Pharmaceuticals, Australia) for 3 s, then quickly dropped onto one of the few fibroblast cells individually that were sedimented in a T2 drop. After attachment, 10 cytoplast-fibroblast cell pairs were equilibrated in fusion medium (0.3 M mannitol and 0.01% PVA) for 10 s. Using an alternative current (AC) of 0.6 KV/cm and 700 KHz, cell pairs were aligned to the wire of a fusion chamber (BTX microslide 0.5 mm fusion chamber, model 450; BTX, SanDiego, Calif., USA) with the donor cells farthest from the wire (FIG. 24b), then fused with a direct current (DC) of 2.0 KV/cm for 9 μs. After the electrical pulse, cell pairs were removed carefully from the wire, transferred to T10 drops and incubated to observe whether fusion had occurred.

Approximately 1 hour after the first fusion, fused pairs together with the remaining two thirds of cytoplasts were equilibrated in activation medium drops separately (0.3 M mannitol, 0.1 mM MgSO₄, 0.1 mM CaCl₂ and 0.01% polyvinylalcohol (PVA)). Under a 0.6 KV/cm AC, cytoplast—fused pair—cytoplast triplets were aligned sequentially to the wire in groups of 10, with fused pairs located in the middle (FIG. 24c). A single DC pulse of 0.7 KV/cm for 80 μs was used for the second fusion and initiation of activation. The triplets were then removed from the wire and transferred carefully to T10 drops to check the fusion (FIG. 24d). Reconstructed embryos were incubated in culture medium (see Embryo culture and evaluation) supplemented with 5 μg/ml cytochalasin B and 10 μg/ml cycloheximide for 4 h at 38.5° C. in 5% CO₂, 5% O₂ and 90% N₂ with maximum humidity, then washed thoroughly for 3 times in IVC medium before culture.

### Parthenogenetic Activation (PA)

Parthenogenetically activated oocytes were produced either separately or in parallel with HMC. Oocytes were denuded in the same way as above except that a longer incubation in pronase was used to get the zona pellucida completely removed. Zona free (ZF) oocytes were then equilibrated for 10 s in activation medium (0.3 M mannitol, 0.1 mM MgSO₄, 0.1 mM CaCl₂ and 0.01% PVA) and transferred to the fusion chamber (BTX microslide 0.5 mm fusion chamber, model 450; BTX, SanDiego, Calif., USA). A single DC pulse of 0.85 KV/cm for 80 μs was generated with a BLS CF-150/B cell fusion machine (BLS, Budapest, Hungary) and applied to ZF oocytes. For zona intact (ZI) oocytes, a single DC pulse of 1.25 KV/cm for 80 μs was used (according to our unpublished preliminary experiments, these parameters resulted in the highest activation and subsequent in vitro development for ZI and ZF oocytes, respectively). The procedure after the electrical pulse was the same as for HMC reconstructed embryos.

### Embryo Culture and Evaluation

All porcine embryos produced by the above treatments were cultured in a modified NCSU37 medium (Kikuchi, et al., 2002) containing 4 mg/ml BSA at 38.5° C. in 5% O₂, 5% CO₂ and 90% N₂ with maximum humidity. The culture medium was supplied with 0.17 mm sodium pyruvate and 2.73 mm sodium lactate from Day 0 (the day for fertilization and activation) to Day 2, then sodium lactate and sodium pyruvate was replaced with 5.5 mm glucose from Day 2 to Day 7. All ZF embryos were cultured in the WOW system (Vajta, et al., 2000) in the same culture medium and gas mixture as used above, with careful medium change on Day 2 without removing the embryos from the WOWs. The blastocyst rate was registered on Day 7. To determine total cell numbers, blastocysts were fixed and mounted to a glass microscopic slide in glycerol containing 20 μg/μl Hoechst 33342 fluorochrome. After staining for 24 h, embryos were observed under a Diaphot 200 inverted microscope with epifluorescent attachment and UV-2A filter (Nikon, Tokyo, Japan).

### Example 1

Differences in developmental competence between sow (2.5 years, 170 Kg in weight) derived oocytes and gilt (5.5˜6 months, 75 Kg in weight) derived oocytes were investigated through ZF and ZI PA after 44 h in vitro maturation. Four combined groups were investigated in 3 identical replicates: (1) ZF oocytes from sows (2) ZI oocytes from sows (3) ZF oocytes from gilts (4) ZI oocytes from gilts. For ZF activation, a single DC pulse of 0.85 KV/cm for 80 μs was applied, while a single 1.25 KV/cm pulse was used to activate ZI oocytes. Following 7 days culture as described above, the percentage of blastocysts per activated embryo was determined.

The in vitro developmental competence of parthenogenetically activated oocytes derived from either sows or gilts was investigated. As shown in Table 1, the blastocyst rates of parthenogenetically activated oocytes from sows were significantly higher than those from gilts, either after ZF or ZI PA.

TABLE 1
Blastocyst development of Day 7 parthenogenetically activated
sow and gilt oocytes

| | Zona Free | | Zona Intact | |
|---|---|---|---|---|
| | No. of | | No. of | |
| | activated | No. of | activated | No. of |
| | oocytes | blastocysts (%)* | oocytes | blastocysts (%)* |
| sow | 103 | 43 (42 ± 4)^{a} | 110 | 61 (55 ± 6)^{c} |
| gilt | 85 | 17 (20 ± 2)^{b} | 137 | 36 (26 ± 5)^{d} |

^{a,b}Different superscripts mean significant differences (p < 0.05).
^{c.d}Different superscripts mean significant differences (p < 0.05).
*Percentage (Mean ± S.E.M) of embryos developed to blastocysts.

The difference in oocytes developmental competence between sows and gilts has been examined in in vitro production (IVP) and somatic cell nuclear transfer (SCNT) embryos separately, resulting in a similar conclusion as in the earlier publication of other research groups (Sherrer, et al., 2004; Hyun, et al., 2003), i.e. that embryos from sow-derived oocytes are superior to those from gilt-derived oocytes in supporting blastocyst development. Although gilts used in our study were at the borderline of maturity, the difference between Day 7 blastocyst rates after PA was significant, proving the superior developmental competence of sow oocytes.

### Example 2

The feasibility of modified porcine HMC was investigated in 6 identical replicates, with IVF and in parallel ZF PA as controls. The more competent sow oocytes (according to Example 1) were used in Example 2. Seven days after reconstruction and/or activation, the number of blastocysts per reconstructed embryo and total cell numbers of randomly selected blastocysts were determined.

More than 90% of oocyte fragments derived from morphologically intact oocytes could be recovered for HMC after the trisection. In average, 37 embryos could be reconstructed out of 100 matured oocytes. The developmental competence of all sources of porcine embryos is shown in Table 2. On Day 7, the development of reconstructed embryos to the blastocyst stage was 17±4% with mean cell number of 46±5, while the blastocyst rates for IVF, and ZF PA were 30±6% and 47±4% (n=243, 170, 97) respectively.

TABLE 2
In vitro development of embryos produced by HMC, IVF and ZF PA

| | No. of | | blastocyst | Mean cell |
|---|---|---|---|---|
| Embryo | embryos/oocytes | No. of | rates (Mean ± | number of |
| origins | in culture | blastocysts | S.E.M). | blastocysts |
| HMC | 243 | 41 | 17 ± 4^{a} | 46 ± 5^{d} |
| IVF | 170 | 52 | 30 ± 6^{b} | 74 ± 6^{e} |
| ZF PA | 97 | 46 | 47 ± 4^{c} | 53 ± 7^{d} |

^{a,b,c}Different superscripts mean significant differences (p < 0.05).
^{d,e}Different superscripts mean significant differences (p < 0.05).

Although the theoretical maximum efficiency was still not approached, the integration of zona partial digestion and oocyte trisection almost doubled the number of reconstructed embryos compared to our earlier system (Kragh, et al., 2004 Reprod. Fertil. Dev 16, 315-318). This increase in reconstruction efficiency may have special benefits in porcine cloning since oocyte recovery after aspiration is more demanding and time-consuming than in cattle. An even more important point is the high embryo number required for establishment of pregnancies following porcine nuclear transfer. IVC in pigs is also regarded as a demanding and inefficient procedure (Reed, et al., 1992 Theriogeneology 37, 95-109). A disadvantage of ZF systems is that the embryos have to reach at least the compacted morula or early blastocyst stage in vitro to avoid disintegration in the oviduct without the protective layer of the zona pellucida. On the other hand, once in the blastocyst stage, zona free embryos can be transferred successfully as proved by calves born after either embryonic or somatic cell nuclear transfer (Peura et al., 1998; Tecirlioglu et al., 2004; Oback et al., 2003; Vajta, et al., 2004) and also by the piglets born after zona-free IVP of oocytes (Wu, et al., 2004). NCSU37 medium has been the most widely and successfully used medium for the culture of pig embryos. However, despite the improved embryo development compared with other media, the viability of IVP porcine embryos is still compromised after IVC. Some reports suggested that glucose is not metabolized readily by early porcine embryos before the eight-cell stage but used in higher amounts in embryos between the compacted morula and blastocysts stages (Flood, et al., 1988). The replacement of glucose with pyruvate and lactate in NCSU37 for the first 2 days culture resulted in a blastocyst rate of 25.3% for IVP porcine embryos in Kikuchi's study (Kukuchi, et al., 2002), which was further corroborated by our present studies with an IVP blastocysts rate of 30% in average. Moreover, the first evaluation of this sequential culture system on porcine HMC and ZF PA embryos has resulted in blastocyst rates of 17% and 47% respectively. Sometimes, the blastocyst rate of ZI PA could even reach levels as high as 90% (Du, unpublished)

### Statistical Analysis

ANOVA analysis was performed using SPSS 11.0. A probability of P<0.05 was considered to be statistically significant.

### Example 3

Vitrification of hand-made cloned porcine blastocysts produced from delipated in vitro matured oocytes.

Recently a noninvasive procedure was published for delipation of porcine embryos with centrifugation but without subsequent micromanipulation (Esaki et al. 2004 Biol Reprod. 71, 432-6).

Cryopreservation of embryos/blastocysts was carried out by vitrification using Cryotop (Kitazato Supply Co, Fujinomiya Japan) as described previously (Kuwayama et al. 2005a; 2005b). At the time of vitrification, embryos/blastocysts were transferred into equilibration solution (ES) consisting of 7.5% (V/V) ethylene glycol (EG) and 7.5% dimethylsulfoxide (DMSO) in TCM199 supplemented with 20% synthetic serum substitute (SSS) at 39° C. for 5 to 15 min. After an initial shrinkage, embryos regained their original volume. 4-6 embryos/blastocysts were transferred into 20 ul drop of vitrification solution (VS) consisting of 15% (V/V) EG and 15% (DMSO) and 0.5M sucrose dissolved in TCM199 supplemented with 20% SSS. After incubation for 20 s, embryos were loaded on Cryotop and plunged into liquid nitrogen. The process from exposure in VS to plunging was completed with 1 min.

Embryos/blastocysts were thawed by immersing Cryotop directly into thawing solution (TS) consisting of 1.0M sucrose in TCM199 plus 20% SSS for 1 min, then transferred to dilution solution (DS) consisting of 0.5 M sucrose in TCM199 plus 20% SSS for 3 min. To remove cryoprotectant, embryos/blastocysts were kept twice in a washing solution (WS; TCM199 plus 20% SSS), 5 min for each time. Survival of vitrified blastocysts was determined according to reexpansion rates after 24 h recovery in culture medium supplemented with 10% calf serum (CS).

The non-invasive delipation method was applied to in vitro matured porcine oocytes and further development of delipated oocytes after parthenogenetic activation was investigated in 4 identical replicates. Oocytes were randomly separated into delipation and control groups.

For delipation, oocytes were digested with 1 mg/ml pronase in the presence of 50% cattle serum (CS) for 3 min, and washed in Hepes-buffered TCM-199 medium supplemented with 20% CS which results in partial zona pellucida digestion (FIG. 25a). Subsequently 40-50 oocytes were centrifuged (12000×g, 20 min) at room temperature in Hepes-buffered TCM-199 medium supplemented with 2% CS, 3 mg/ml PVA and 7.5 μg/ml cytochalasin B (CB) (FIG. 25b). Zonae pellucidea of both centrifuged and intact oocytes were removed completely with further digestion in 2 mg/ml pronase solution. For activation, a single direct current of 85 Kv/cm for 80 us was applied to both groups, followed by 4 h treatment with 5 μg/ml CB and 10 μg/ml cycloheximide (CHX). All embryos were then cultured in the modified NCSU37 medium. Day 7 blastocysts were vitrified and warmed by using the Cryotop technique (Kuwayama et al., RBM Online, in press) at 38.5° C. Survival of vitrified blastocysts was determined according to reexpansion rates after 24 h recovery in culture medium supplemented with 10% CS. Cell numbers of reexpanded blastocysts from both groups were determined after Hoechst staining. Results were compared by ANOVA analysis. Partial zona digestion and centrifugation resulted in successful delipation in 173/192 (90%) of oocytes. The development to blastocysts was not different between delipated and intact oocytes (28±7% vs. 28±5% respectively; P>0.05). However, survival rates of blastocysts derived from delipated oocytes were significantly higher than those developed from intact oocytes (85±6% vs. 32±7% respectively; P<0.01). There is no difference in average cell number of reexpanded blastocysts derived from either delipated or intact oocytes (36±7 vs. 38±9, respectively; P>0.05). The results demonstrate that the simple delipation technique does not hamper the in vitro development competence of activated porcine oocytes, and improves the cryosurvival of the derived blastocysts without significant loss in cell number.

After delipation, zona pellucida of oocytes from both groups was removed completely. The same parameters as described above for electrical activation were applied to both groups. Seven days after activation, blastocyst rates and blastocyst cell numbers were determined.

The feasibility of applying a non-invasive delipation technique to in vitro matured porcine oocytes was investigated. 90% (173/192) oocytes can be delipated successfully. As shown in table 3, the development to blastocysts was not different between delipated and intact oocytes (28±7% vs. 28±5% respectively; P>0.05). However, survival rates of blastocysts derived from delipated oocytes were significantly higher than those developed from intact oocytes (85±6% vs. 32±7% respectively; P<0.01). There is no difference in average cell number of reexpanded blastocysts derived from either delipated or intact oocytes (36±7 vs. 38±9, respectively; P>0.05).

TABLE 3
Developmental competence and cryosurvival of vitrified-thawed
embryos from delipated and intact activated oocytes.

| | | | | Mean cell |
|---|---|---|---|---|
| | | | Reexpanded | number |
| Oocyte | Activated | Blastocyst rate | blastocyst after | of reexpanded |
| treatment | oocyte | (%) | warming (%) | blastocysts |
| Delipated | 173 | 28 ± 7 | 85 ± 6 | 36 ± 7 |
| Intact | 156 | 28 ± 5 | 32 ± 7 | 39 ± 9 |

### Handmade Cloning of Delipated Oocytes

Delipated oocytes were used for HMC in 5 replicates. Four identical replicates of non-delipated oocytes for HMC were used as a control system. Seven days after reconstruction, blastocysts produced from both groups were vitrified with Cryotop. Survival rates and cell numbers of re-expanded blastocysts were determined as described for the blastocysts produced by PA.

Except where otherwise indicated, all manipulations were performed on a heated stage adjusted to 39° C., and all drops used for handling oocytes were of 20 μl volume covered with mineral oil. For somatic cell nuclear transfer, the handmade cloning (HMC) described in our previous work (Du, et al., 2005) was applied with a single modification: for enucleation of both delipated and control oocytes, bisection instead of trisection was applied.

Briefly, after the removal of cumulus investment, control oocytes were incubated in 3.3 mg/ml pronase dissolved in T33 for 10 s. Before the oocytes started to become misshaped in pronase solution, they were picked out and washed quickly in T2 and T20 drops. Delipated oocytes after centrifugation were digested in the 3.3 mg/ml pronase solution for an additional 5 s.

Both control and delipated oocytes with partially digested, distended and softened zonae pellucidae were lined up in T2 drops supplemented with 2.5 μg/ml cytochalasin B. Bisection was performed manually under stereomicroscopic control (FIG. 25c) with Ultra Sharp Splitting Blades (AB Technology, Pullman, Wash., USA). Halves were collected and stained with 5 μg/ml Hoechst 33342 fluorochrome in T2 drops for 5 min, and then placed into 1 μl drops of T2 medium on the bottom of a 60 mm Falcon Petri dish covered with oil (3-4 halves per drop). Using an inverted microscope and UV light, positions of halves without chromatin staining (cytoplasts) were registered. Cytoplasts were later collected under a stereomicroscope and stored in T10 drops.

Porcine foetal fibroblast cells were prepared with trypsin digestion from monolayers as described previously (Kragh, et al., 2005). Fusion was performed in two steps where the second one included the initiation of activation, as well. For the first step, 50% of the available cytoplasts were transferred into 1 mg/ml of phytohaemagglutinin (PHA; ICN Pharmaceuticals, Australia) dissolved in TO for 3 s, then quickly dropped over single fibroblast cells. After attachment, cytoplast-fibroblast cell pairs were equilibrated in fusion medium (0.3 M mannitol and 0.01% PVA) for 10 s and transferred to the fusion chamber. Using an alternating current (AC) of 0.6 KV/cm and 700 KHz, pairs were aligned to the wire of a fusion chamber with the somatic cells farthest from the wire (FIG. 25d), then fused with a direct current of 2.0 KV/cm for 9 μs. After the electrical pulse, cell pairs were removed carefully from the wire, transferred to T10 drops and incubated to observe whether fusion had occurred.

Approximately 1 hour after the first fusion, each pair was fused with another cytoplast in activation medium. AC current and a single DC pulse of 0.7 KV/cm for 80 μs were applied as described above. Fusion was detected in T10 drops, then reconstructed embryos were transferred into IVC0-2 medium (see Embryo culture and evaluation) supplemented with 5 μg/ml cytochalasin B and 10 μg/ml cycloheximide. After a 4 h incubation at 38.5° C. in 5% CO₂, 5% O₂ and 90% N₂ with maximum humidity, embryos were washed 3 times in IVC0-2 medium before culture.

TABLE 4
Developmental competence and cryosurvival of vitrified-thawed
embryos of SCNT porcine embryos derived from delipated and
intact oocytes.

| | | | | Mean cell |
|---|---|---|---|---|
| | No. of | | Reexpanded | number of |
| HMC | reconstructed | Blastocyst | blastocyst after | reexpanded |
| group | embryos | rate (%)* | warming (%)* | blastocysts* |
| Delipated | 240 | 21 ± 6^{a} | 79 ± 6^{b} | 41 ± 7^{d} |
| Intact | 150 | 23 ± 6^{a} | 32 ± 8^{c} | 39 ± 5^{d} |

Different superscripts mean significant differences (p < 0.05).
*mean ± S.E.M.

In vitro developmental competence was observed in HMC with delipated oocytes when Day 7 blastocyst rates were compared with control HMC group (21±6% vs. 23±6% respectively; P>0.05; Table 4). Cryosurvival rate after vitrification of cloned blastocysts derived from delipated oocytes was significantly higher than those developed from intact oocytes (79±6% vs. 32±8, respectively; P<0.01).

### Example 4

### Chemically Assisted Handmade Enucleation (CAHE) and Comparison to Existing Methods

After 41-42 h maturation in vitro, COCs were further cultured for 45 min in the same solution supplemented by 0.4 μg/ml demecolcine. Cumulus cells were then removed by pipetting in 1 mg/ml hyaluronidase dissolved in Hepes-buffered TCM-199. From this point (except where otherwise indicated), all manipulations were performed on a heated stage adjusted to 39° C. All drops used for handling oocytes were of 20 μl in volume, and were covered with mineral oil.

Basic steps of the HMC procedure have been described elsewhere herein. Briefly, oocytes without cumulus cells were incubated in 3.3 mg/ml pronase dissolved in T33 (T for Hepes-buffered TCM 199 medium; the number means percentage [v/v] of CS supplement, here 33%) for 20 s. When partial lyses of zonae pellucidae and slight deformation of oocytes occurred, they were picked up and washed quickly in T2 and T20 drops. Nine oocytes were lined up in one T2 drop supplemented with 2.5 μg/ml cytochalasin B (CB). By using a finely drawn and fire-polished glass pipette, oocytes were rotated to find a light extrusion cone and/or strongly attached polar body on the surface, and oriented bisection was performed manually under stereomicroscopic control with a microblade (AB Technology, Pullman, Wash., USA). Less than half of the cytoplasm (close to the extrusion or PB) was separated from the remaining part (FIG. 26). After bisection of all 9 oocytes in the drop, larger parts and smaller parts (with the extrusion or attached PB) were collected and placed into separate drops of T2, respectively.

Oriented Handmade Enucleation without Demecolcine Treatment (OHE)

All steps were similar to the previously described procedure, but demecolcine preincubation was not applied.

### Random Handmade Bisection for Enucleation (RHE)

Demecolcine preincubation was omitted from the pretreatment of this group, as well. After removal of cumulus cells, zonae pellucidae were partially digested by pronase as described above. Random handmade equal bisection was applied in drops of T2 supplemented with 2.5 μg/ml CB. All demi-oocytes were selected and stained with 10 μg/ml Hoechst 33342 in T2 drops for 10 min, then placed into 1 μl drops of T2 medium covered with mineral oil (three demi-oocytes into each drop). Using an inverted microscope and UV light, the positions of chromatin free demi-oocytes, i.e. cytoplasts were registered. These cytoplasts were later collected under a stereomicroscope and stored in T2 drops before further manipulations.

### Fusion and Initiation of Activation

Porcine fetal fibroblast cells were prepared as described previously (Kragh, et al., 2005, Du, et al., 2005). Fusion was performed in two steps, where the second one included the initiation of activation as well. For the first step, with a finely drawn and fire-polished glass pipette, approximately 100 somatic cells were placed into a T2 drop, and 20-30 cytoplasts were placed into a T10 drop. After a short equilibration, groups of 3 cytoplasts were transferred to 1 mg/ml of phytohaemagglutinin (PHA) for 2-3 sec, then each was quickly dropped over a single somatic cell. Following attachment, cytoplast-somatic cell pairs were picked up again and transferred to a fusion medium (0.3 M mannitol supplemented with 0.01% [w/v] PVA). By using an alternative current (AC) of 0.6 KV/cm and 700 KHz, equilibrated pairs were aligned to one wire of a fusion chamber (BTX microslide 0.5 mm fusion chamber, model 450; BTX, San Diego, Calif.) with the somatic cells farthest from the wire, then fused with a single direct current (DC) impulse of 2.0 KV/cm for 9 μsec. Pairs were then removed carefully from the wire to a T10 drop, and incubated further to observe whether fusion had occurred.

Approximately 1 h after the fusion, fused pairs and the remaining cytoplasts were separately equilibrated in activation medium (0.3 M mannitol, 0.1 mM MgSO₄, 0.1 mM CaCl₂, supplemented with 0.01% [w/v] PVA). By using a 0.6 KV/cm AC, one pair and one cytoplast was aligned to one wire of the fusion chamber, with fused pairs contacting the wire. A single DC pulse of 0.86 KV/cm for 80 μsec was used for the second fusion and initiation of activation. Fusion was checked in after incubation in T10 drops.

### Traditional Cloning (TC)

Micromanipulation was conducted with a Diaphot 200 inverted microscope (Nikon, Tokyo, Japan), as described before (Chen et al., 1999; Zhang et al., 2005). Briefly, after 42-44 h in vitro maturation, the cumulus cells were removed as described above. All manipulations were performed on a heated stage adjusted to 39° C. A single 50 μL micromanipulation solution drop was made in the central area on a lid of 60 mm culture dish and covered with mineral oil. Groups of 20-30 oocytes and fetal fibroblast cells were placed in the same drop. After incubation for 15-30 min, the oocyte was secured with a holding pipette (inner diameter=25-35 μm and outer diameter=80-100 μm). After being placed at the position of 5-6 o'clock, the first polar body and the adjacent cytoplasm (approx. 10% of the total volume of the oocyte) presumptively containing metaphase plate were aspirated and removed with a beveled injection pipette (inner diameter=20 μm). A fetal fibroblast cell was then injected into the space through the same slit. After nuclear transfer (NT), reconstructed couplets were transferred into drops of media covered with mineral oil for recovery for 1-1.5 h until fusion and activation was conducted. The recovery medium was NCSU-23 supplemented with 4 mg/mL BSA and 7.5 μg/mL CB. Reconstructed couplets were incubated in fusion medium for 4 min. Couplets were aligned manually using a finely pulled and polished glass capillary to make the contact plane parallel to electrodes. A single, 30 μsec, direct current pulse of 2.0 kV/cm was then applied. After culture in drops of IVC0-2 (specified in “Embryo culture and evaluation”) supplemented with 7.5 μg/mL CB for 30-60 min, fusion results were examined under a stereomicroscope. Fused couplets were subjected to a second pulse in activation solution. After 30 min incubation in T10 they were transferred to IVC0-2 to evaluate in vitro development.

### Further Steps of Activation

After the activation impulse, all reconstructed embryos were incubated in IVC0-2 supplemented with 5 μg/ml CB and 10 μg/ml cycloheximide at 38.5° C. in 5% CO₂, 5% O₂, and 90% N₂, with maximum humidity.

### Embryo Culture and Evaluation

4 h later, all reconstructed and activated embryos were washed and cultured in Nunc four-well dishes in 400 μl IVC0-2 covered by mineral oil at 38.5° C. in 5% CO₂, 5% O₂, and 90% N₂, with maximum humidity. IVC0-2 was a modified NCSU37 medium (Kikuchi, et al., 1999), containing 4 mg/ml BSA, 0.17 mM sodium pyruvate, and 2.73 mM sodium lactate from Day 0 (the day for activation) to Day 2. Sodium pyruvate and sodium lactate were replaced with 5.5 mM glucose from Day 2 to Day 7 (IVC2-7). All zonae free embryos were cultured in the Well of the Well (WOW) system (Vajta et al., 2000) in the same culture medium and gas mixture as used above, with careful medium change on Day 2 without removing the embryos from the WOWs. TC embryos were cultured in groups of 15 to 30 in wells of four-well dishes by using the same medium amount and composition. Cleavage and blastocyst rates were registered on Day 2 and Day 7, respectively. To determine total cell numbers, blastocysts were fixed and mounted to a glass microscope slide in a small amount (<2 μl) of glycerol containing 10 μg/ml Hoechst 33342. After staining for several hours at room temperature, embryos were observed under a Diaphot 200 inverted microscope with epifluorescent attachment and UV-2A filter (Nikon, Tokyo, Japan).

Comparison of Efficiency of CAHE vs. OHE

The efficiency and reliability of CAHE was tested in 12 identical replicates by using a total of 620 oocytes. After 41-42 h maturation, oocytes were subjected to demecolcine incubation. Oriented bisection was performed in oocytes where an extrusion cone and/or a strongly attached PB was detected after partial pronase digestion. Percentages of bisected vs. total oocytes and surviving vs. bisected oocytes were registered. Subsequently both putative cytoplasts and karyoplasts were collected separately and stained with Hoechst 33342 (10 μg/ml in T2 for 10 min). The presence or absence of chromatin was detected under an inverted fluorescent microscope (FIG. 27).

The efficiency and reliability of OHE was investigated in 9 identical replicates using a total of 414 oocytes. After 42-43 h in vitro maturation, oriented bisection was performed in matured oocytes where an extrusion cone and/or a PB was detected after partial pronase digestion. Results were evaluated as described in the previous paragraph.

The results are shown in Table 5.

TABLE 5
The efficiency of chemically assisted handmade enucleation
(CAHE) and oriented handmade enucleation (OHE)

| | | | Cytoplast/ | |
|---|---|---|---|---|
| | No. of treated | Bisected/total | bisection | Cytoplast/total |
| Groups | oocytes | oocytes (%)* | (%)* | oocyte (%)* |
| CAHE | 620 | 96 ± 1^{a} | 94 ± 2^{b} | 90 ± 3^{c} |
| OHE | 414 | 92 ± 2^{a} | 88 ± 3^{b} | 81 ± 4^{d} |

*mean ± A.D. (absolute deviations)
Different superscripts mean difference (P < 0.05)

No differences between groups regarding extrusion cones and/or attached polar bodies allowing oriented bisection or in the lysis rates were detected, and the successful enucleation per bisected oocyte ratio was also similar. However the overall efficiency of the procedure measured by the cytoplast per total oocyte number was higher in the CAHE than in the OHE group.

Comparison of in vitro development of embryos produced with CAHE, RHE and TC

In 8 replicates, a total of 468 in vitro matured oocytes were randomly distributed and subjected to three of the enucleation procedures described above. Fusion rates between cytoplast and donor fibroblasts were registered. Reconstructed embryos were activated and cultured as described earlier. Cleavage and blastocyst rates were determined on Day 2 and Day 7, respectively. Stereomicroscopic characteristics of the developed blastocysts were compared between groups.

TABLE 6
Developmental competence of embryos derived from chemically assisted
handmade enucleation (CAHE), random handmade enucleation (RHE) and
traditional, micromanipulator based cloning (TC).

| | No. of | | | | Cell no. of |
|---|---|---|---|---|---|
| | reconstructed | Fusion rate | Cleavage | Blastocyst | blastocysts |
| Groups | embryos | (%)* | rate (%)* | rate (%)* | (Day 7) |
| CAHE | 150 | 87 ± 7^{a} | 97 ± 6^{b} | 28 ± 9^{d} | 57 ± 6^{e} |
| RHE | 86 | 81 ± 4^{a} | 87 ± 8^{b} | 21 ± 9^{d} | 49 ± 7^{e} |
| TC | 178 | 81 ± 10^{a} | 69 ± 9^{c} | 21 ± 6^{d} | 53 ± 6^{e} |

*mean ± A.D. (absolute deviations)
Different superscripts mean difference (P < 0.05).

Fusion rates after enucleation were similar between CAHE, RHE and TC, respectively. The second fusion and activation resulted in negligible (<1%) losses in the first two groups. Although TC resulted in lower cleavage per reconstructed embryo rates than the other two groups, this difference was not present in the blastocyst per reconstructed embryo rates.

Stereomicroscopic characteristics (size; estimated proportion and outlines of the inner cell mass) did not differ between groups. Cell numbers (57±6 vs. 49±7 vs. 53±6) of the produced blastocysts from CAHE, RHE and TC are shown in Table 6, FIG. 28 and FIG. 29.

### Statistical Analysis

AVEDEV was performed by Microsoft XP Excel software and ANOVA was performed by SAS system. A probability of P<0.05 was considered to be statistically significant.

### Example 5

### Production of Piglets

### Handmade Cloning (HMC)

Forty one hrs after the start of in vitro maturation, the cumulus investment of the COCs was removed by repeated pipetting in 1 mg/ml hyaluronidase in Hepes-buffered TCM199. From this point (except where otherwise indicated) all manipulations were performed on a heated stage adjusted to 39° C., and all drops used for handling oocytes were of 20 μl volume covered with mineral oil. Oocytes were briefly incubated in 3.3 mg/ml pronase dissolved in T33 (T for Hepes-buffered TCM 199 medium; the number means percentage (v/v) of calf serum (CS) supplement, here 33%) for 20 sec and then quickly washed in T2 and T20 drops. Oocytes with partially digested but still visible zona were lined up in drops of T2 supplemented with 2.5 μg/ml cytochalasin B (CB). With a finely drawn and fire-polished glass pipette, oocytes were rotated to find the polar body (PB) on the surface, and oriented bisection was performed manually under stereomicroscopic control with a microblade (AB Technology, Pullman, Wash., USA). Thus, less than half of the oocyte cytoplasm (close to the extrusion or PB) was removed from the remaining putative cytoplast. Cytoplasts were washed twice in T2 drops and collected in a T10 drop.

Fetal fibroblast cells were prepared as described previously (Kragh, P. M. et al. Theriogenology 64, 1536-1545 (2005).

Fusion was performed in two steps where the second one included the initiation of activation, as well. For the first step, halves of putative cytoplasts were used. With a finely drawn and fire-polished glass pipette, 10 cytoplasts were transferred as a group to 1 mg/ml of phytohaemagglutinin (PHA; ICN Pharmaceuticals, Australia) for 3 sec, then quickly dropped individually onto one of the few fibroblast cells that were sedimented in a T2 drop. After attachment, 10 cytoplast-fibroblast cell pairs were equilibrated in fusion medium (0.3 M mannitol and 0.01% PVA) for 10 sec. Using an alternative current (AC) of 0.6 KV/cm and 700 KHz, cell pairs were aligned to the wire of a fusion chamber (BTX microslide 0.5 mm fusion chamber, model 450; BTX, SanDiego, Calif., USA) with the somatic cells farthest from the wire, then fused with a direct current (DC) of 2.0 KV/cm for 9 μsec. After the electrical pulse, cell pairs were removed carefully from the wire, transferred to T10 drops and incubated to observe whether fusion had occurred.

Approximately 1 hr after the first fusion, fused pairs together with the remaining cytoplasts were equilibrated in activation medium drops separately (0.3 M mannitol, 0.1 mM MgSO₄, 0.1 mM CaCl₂ and 0.01% PVA). Under a 0.6 KV/cm AC, cytoplast-fused pair were aligned sequentially to the wire in groups of 10, with fused pairs far from the wire. A single DC pulse of 0.7 KV/cm for 80 μsec was used for the second fusion and initiation of activation. The pairs were then removed from the wire and transferred carefully to T10 drops to check the fusion. Reconstructed embryos were incubated in PZM-3 medium supplemented with 5 μg/ml CB and 10 μg/ml cycloheximide for 4 hr at 38.5° C. in 5% CO₂, 5% O₂ and 90% N₂ with maximum humidity, then washed thoroughly before culture.

### Traditional Cloning (TC)

Micromanipulation was conducted with a Diaphot 200 inverted microscope (Nikon, Tokyo, Japan). Cumulus cells were removed as described above after 42 to 44 hr maturation. All manipulations were performed on a heated stage adjusted to 39°. A single 50 μL drop of micromanipulation solution (NCSU-23 supplemented with 4 mg/mL BSA and 7.5 μg/mL CB) was made in the central area on a lid of 60 mm culture dish and covered with mineral oil. Groups of 20 to 30 oocytes and fetal fibroblast cells were placed in the same drop. After incubation for 15 to 30 min, one oocyte was secured with a holding pipette (inner diameter=25-35 μm and outer diameter=80-100 μm). After being placed at the position of 5-6 o'clock, the first polar body and the adjacent cytoplasm (approx. 10% of the total volume of the oocyte) presumptively containing metaphase plate were aspirated and removed with a beveled injection pipette (inner diameter=20 μm). A fetal fibroblast cell was then injected into the space through the same slot. After nuclear transfer (NT), reconstructed couplets were transferred into drops of media covered with mineral oil for recovery for 1 to 1.5 hrs until fusion and activation was conducted. Reconstructed couplets were incubated in fusion medium for 4 min. Couplets were aligned manually using a finely pulled and polished glass capillary to make the contact plane parallel to electrodes. A single, 30 μsec, direct current pulse of 2.0 kV/cm was then applied. After culture in drops of PZM-3 medium supplemented with 7.5 μg/mL CB for 30-60 min, fusion results were examined under a stereomicroscope. Fused couplets were subjected to a second pulse in activation solution. After 30 min incubation in T10 they were transferred to PZM-3 medium to evaluate in vitro development.

### Embryo Culture and Transfer

Reconstructed embryos were cultured in PZM-3 medium (Dobrinsky, J. T. et al. Biol Reprod 55, 1069-1074 (1996) supplemented with 4 mg/ml BSA. Zona-free embryos produced from HMC were cultured in the modified WOWs system (Feltrin, C. Et al. Reprod Fertil Dev 18, 126 (2006). Two different cell lines (LW1-2 for HMC, LW2 for TC) were used as nuclear donor cells for HMC and TC to allow the identification of the offspring from the two procedures. LW1-2 and LW2 originate from fetuses from a cross (with Duroc) and pure Danish landrace, respectively.

The average blastocyst per reconstructed embryo rate after in vitro culture for 7 days was 50.1±2.8% (mean±S.E.M), which is significantly higher (p<0.01) for HMC than that of TC performed in parallel in our laboratory (Table 7) and also the highest one that has ever been reported in pig cloning.

TABLE 7
In vitro development of embryos produced from handmade
cloning and traditional cloning

| | | No. of | | |
|---|---|---|---|---|
| | Somatic cell | reconstructed | Cleavage rate | Blastocyst rate |
| Group | donor | embryos | (%) | (%) |
| HMC | LW1-2 | 643 | 83.7 ± 4.90^{a} | 50.06 ± 2.80^{a} |
| TC | LW2 | 831 | 74.86 ± 13.16^{b} | 28.98 ± 2.84^{b} |

^{a,b}Values of different superscripts within columns are significantly different (p < 0.05).
*mean ± S.E.M.

Mixed blastocysts produced from both HMC and TC were surgically transferred to 11 naturally synchronized sows on Day 4 or 5 of estrous cycle. Six (55%) recipients were diagnosed pregnant by ultrasonography, 2 aborted and by the time of writing 2 have delivered 3 and 10 piglets, respectively. A litter size of 10 cloned piglets is, according to our knowledge, the largest litter size so far achieved in pig cloning. All of them are healthy and behave normally except one showed rigid flexure of distal joint of one foreleg. %).

Preliminary results suggest that when embryos of similar stages were transferred, recipients on Day 4 of the estrous cycle supported pregnancy establishment better than those of Day 5 (Table 8).

TABLE 8
In vivo development of cloned porcine embryos

| | | | | | | No. of piglets | | |
|---|---|---|---|---|---|---|---|---|
| | Embryos | | | | | born | | |
| | transferred | | Embryo | Recipient | | piglets | | Gestation |
| Recipient | HMC | TC | stage | cycle | Pregnancy | from | No. piglets | length |
| number | embryo | embryo | (Day) | (Day) | status | HMC | from TC | (Day) |
| 1327 | 22 | 10 | D5, 6, 7 | 4 | Y | 2 | 1 | 116 |
| 1539 | 36 | 10 | D7 | 4 | Y | 8 | 2 | 115 |
| 1309 | 30 | 28 | D5, 6 | 4 | Y | | | |
| 1553 | 45 | 44 | D5, 6 | 4 | Y | | | |
| 1668 | 48 | 18 | D5, 6 | 5 | Y, aborted | | | |
| 1428 | 78 | 22 | D5, 6 | 5 | Y, aborted | | | |
| 1725 | 44 | 4 | D5, 6, 7 | 5 | N | — | — | — |
| 1643 | 22 | 11 | D5, 6, 7 | 4 | N | — | — | — |
| 1520 | 30 | 26 | D5, 6 | 4 | N | — | — | — |
| 1363 | 37 | 7 | D6, 7 | 5 | N | — | — | — |
| 1560 | 99 | 42 | D5, 6, 7 | 5 | N | — | — | — |

### Microsatellite Analysis

Parental analysis using 10 different porcine microsatellite markers confirmed the identical genotype of cloned piglets and donor cells used for nuclear transfer. Identification was done by microsatellite analysis of genomic DNA from each of the newborn piglets, the surrogate sow, and the donor skin fibroblasts LW1-2 and LW2 originating from two fetuses that represent Danish landrace and Duroc, respectively. Ten polymorphic microsatellite loci (SW886, SW58, SW2116, SW1989, SW152, SW378, KS139, SO167, SW1987, SW957) located on different porcine chromosomes were amplified by 3-color multiplex PCR and the products analyzed on the Genetic Analyzer 3130 X1 (Applied Biosystems) using the program Gene Mapper 3.7.

For the second recipient, the offspring per embryo rate (22%) was the highest one ever reported so far in pig cloning (Walker, S. C. et al. Cloning Stem Cells 7, 105-112 (2005); Hoshino, Y. et al. Cloning Stem Cells 7, 17-26 (2005)). Comparable live birth/transferred embryo efficiencies were obtained in HMC (17%) and TC (15%).

### Statistical Analysis

Differences between the experimental groups were evaluated using independent-samples t-test by SPSS 11.5. P<0.05 was considered significant.

### Sequences

### Breast Cancer

```
SEQ ID NO.: 1
tttngtatgctgaaacttctcaaccagaagaaagggccttcacagT > Ggtcctttgtgtaagaatgatataaccaaaagg
SEQ ID NO.: 2
Porcine BRCA 2 gene region of exon 11
      1 ggtccaggat gtttctcttc aagcaaatgt aatgattctg atgtttcaat atttaaggta
    61 gaaaattata gcagtgataa aagtttaagt gagaaataca ataaatgcca actgatacta
  121 aaaaataaca ttgaaaggac tgctgacatt tttgttgaag aaaatactga cggttacaag
  181 agaaatactg aaaataaaga caacaaatgt actggtcttg ctagtaactt aggaggaagc
  241 tggatggaca gtgcttcaag taaaactgat acagtttata tgcacgaaga tgaaactggt
  301 ttgccattta ttgatcacaa catacatcta aaattaccta accactttat gaagaaggga
  361 aatactcaaa ttaaagaagg tttgtcagat ttgacttgtt tggaagttat gagagccgaa
  421 gaaacatttc atattaatac atcaaataaa cagtcaactg ttaataagag gagccaaaaa
  481 ataaaagatt ttgatgtttt tgatttgtcc tttcagagtg caagtgggaa aaacatcaga
  541 gtctctaaag agtcattaaa taaagctgta aatttctttg acgaaaaatg cacagaagaa
  601 gaattgaata acttttcaga ttcctcaaat tctgaaatac ttcctggcat aaatatcaac
  661 aaaataaaca tttcaagcca taaggaaaca gattcggaca aaaacaaact attgaaagaa
  721 agtgacccag ttggtattga aaatcaatta ctgactctcc agcaaagatc agaatgtgaa
  781 atcaaaaaga tcgaagaacc taccatgctg ggttttcata cagctagtgg gaaaaaagta
  841 aaaattgcga aggaatcgtt ggacaaagtg aaaaatcttt ttgatgaaac aaagcaagat
  901 agtagtgaaa ccactaattc tagccatcaa ggggtaaaaa cacagaagga cagagaggta
  961 tgtaaagaag agcttgaatt aacattcgag acagttgaaa taactgcctc aaagcatgaa
1021 gaaatacgga attttttaga ggagaaaaaa cttgtttcta aggagatcac catgccaccc
1081 aggctcttac gtcatcattt acacagacaa actgaaaatc tcagcatgtc aaacagtatc
1141 cccctaaaag gtaaagtaca tgaaaatatg gaagaagaaa catcttgtca cacagatcag
1201 tccacttgtt cagccattga aaattcagca ttaacatttt acacaggaca tggcagaaaa
1261 atttctgtga atcaggcttc cgtatttgaa gccaaaaagt ggcttagaga aggagaattg
1321 gacgatcaac cagaaaacgt agattctgcc aaggtcatat gtttaaagga atatgctagg
1381 gattatgtag gaaatccttt gtgtgggagt agttcaaaca gtatcataac tgaaaatgac
1441 aaaaatctcc ctgaaaaaca aaattcaact tatttaagta acagtgtgtc taacaactat
1501 tcataccatt ctgatttttg tcattccaat gaggtgctca gcaaatcaga atctctctca
1561 gaaaataaaa ttggtaattc tgatactgag ccagcagtga agaatgtcaa agacagaaaa
1621 gacacttgtt tttctgaaga gatatccacc gtaagagaag caaacacaca cccacaagct
1681 gtagatgaag acagctgggt tcggaagctt gtgattaact ctacaccatg caaaaataaa
1741 aatacacctg gtgaagtgtc caatctaatt caaataattt tgagatagag ccacctgcat
1801 tcagtacaag tgggaacata gcctttgttt cacatgaaac agacgtgaga gagaggtttg
1861 cagacaacaa caggaaggcg attaagcaaa acactgagag tatgtcaggc tcttgccaaa
1921 tgaaaattat gactggcgct cataaggcat tgggtgattc agaggatgtt attttcccta
1981 actctccaga tagtgaagaa catattacac gttcacagga ggtttttcct gaaattcaaa
2041 gtgaacaaat tttacaacat gacccaagtg tatccggatt ggagaaagtt tctgaaatgc
2101 caccttgtca tattaactta aaaacttttg atatacataa gtttgatatg aaaagacatc
2161 ccatgtcagt ctcttctatg aatgattgtg gggtttttag cacagcaagt ggaaaatctg
2221 tacaagtatc agatactgca ttacaaaaag cgagacaagt attttctaag acagaagatg
2281 tggctaagcc attcttttcc agagcagtta aaagtgatga agaacattca gacaagtaca
2341 caagagaaga aaatgctatg atgcatcccc ccccaaattt cctgtcatct gctttctccg
2401 gatttagtac agcaagtgga aaacaggttc cagtttctga gagtgcctta tgcaaagtga
2461 agggaatgtt tgaggaattt gatttaatgg gaactgaatg tagacttcag cattcaccta
2521 catctagaca agatgtgtca aagatacttc ctctctccga gattgatgag agaaccccag
2581 aacactctgt aagttcccaa acagagaaag cctacaatga acaatttaaa ttaccagata
2641 gctgtaacac tgaaagcagt tcttcagaaa ataatcactc tgttaaagtt tctcccgatc
2701 tctctcggtt taagcaagac aaacagttgg tatcaggagc aaaagtatca cttgttgaga
2761 acattcatcc atcgggaaaa gaa
SEQ ID NO: 3:
Porcine BRCA 1 gene region of exon 11
    1 agcatgagac cagcagttta ttactcacta aagacagaat gaatgtagaa aaggctgaat
  61 tttgtaataa aagcaagcag cctgtcttag caaagagcca acagagcaga tgggctgaaa
121 gtaagggcac atgtaatgat aggcagactc ctaacacaga gaaaaaggta gttctgaata
181 ctgatctcct gtatgggaga aacgaactga ataagcagaa acctgcgtgc tctgacagtc
241 ctagagattc ccaagatgtt ccttggataa cattgaatag tagcatacag aaagttaatg
301 agtggttttc tagaagcgat gaaatgttaa cttctgacga ctcacaggac aggaggtctg
361 aatcaaatac tggggtagct ggtgcagcag aggttccaaa tgaagcagat ggacatttgg
421 gttcttcaga gaaaatagac ttaatggcca gtgaccctca tggtgcttta atacgtgaac
481 gtgaaagagg gcactccaaa ccagcagaga gtaatattga agataaaata tttgggaaaa
541 cctatcggag gaaggcaagc ctccctaact tgagccacgt aattgaagat ctaattttag
601 gagcatctgc tgtagagcct caaataacac aagagcgccc cctcacaaat aaactaaagc
661 ggaaaaggag aggtacatc
```

### Mitochondria Related Protein Folding Disorders

The sequence is cloned into pN1-EGFP (Clonteq) with a CAGGS promoter and as a fusiogene with EGFP (CAGGS-OTCΔ-EGFP and transfected into porcine fetal fibroblasts:

SEQ ID NO.: 1
Rat Otc-Δ cDNA, deleted nucleotides are underlined.
atggttcgaaattttcggtatgggaagccagtccagagtcaagtacagctgaaaggccgtgacctcctcaccctgaaga
acttcacaggagaggagattcagtacatgctatggctctctgcagatctgaaattcaggatcaaacagaaaggagaata
cttgcctttattgcaagggaaatccttagggatgatttttgagaaaagaagtactcgaacaagactgtccacagaaacag
gcttcgctcttctgggaggacatccttcttttcttaccacacaagacattcacttgggcgtgaatgaaagtctcacagacaca
gctcgtgtgttatctagcatgacagatgcagtgttagctcgagtgtataaacaatcagatctggacatcctggctaaggaag
caaccatcccaattgtcaacggactgtcagacctgtatcatcctatccagatcctggctgattaccttacactccaggaaca
ctatggctctctcaaaggtctcaccctcagctggataggagatgggaacaatatcctgcactccatcatgatgagtgctgc
aaaattcgggatgcaccttcaagcagctactccaaagggttatgagccagatcctaatatagtcaagctagcagagcag
tatgccaaggagaatggtaccaggttgtcaatgacaaatgatccactggaagcagcacgtggaggcaatgtattaatta
cagatacttggataagcatgggacaagaggatgagaagaaaaagcgtcttcaagctttccaaggttaccaggttacaat
gaagactgctaaagtggctgcgtctgactggacgtttttacactgcttgcctagaaagccagaagaagtagatgatgaag
tgttttattctccgcggtcattagtgttcccagaggcagaaaatagaaagtggacaatcatggctgtcatggtatccctgctg
acagactactcacctgtgctccagaagccaaagttctgatgcctgcaagaggacgaaaaacccaaaagacaaaaaa
atctgttctttagcagcagaataagtcagtttatgtagaaaagagaagaattgaaattgtaaacacatccctagtgcgtgat
ataattatgtaattgctttgctattgtgagaattgcttaaagcttttagtttaagtgctgggcattttattatcctgcttgacttgactta
agcactctcttcaattcacaacttctgaatgatatttgggtttcatattaattatcatacacatttccttccactaagcattaaaca
ctatgcttacaatgcataccatctaagtcattaaatgtaatccatgcttattacctt
SEQ ID NO.: 2
Rat Otc-Δ protein, deleted aminoacids are underlined.
M V R N F R Y G K P V Q S Q V Q L K G R D L L T L K N F T G E E I Q Y M L W L S
A D L K F R I K Q K G E Y L P L L Q G K S L G M I F E K R S T R T R L S T E T G F
A L L G G H P S F L T T Q D I H L G V N E S L T D T A R V L S S M T D A V L A R V
Y K Q S D L D I L A K E A T I P I V N G L S D L Y H P I Q I L A D Y L T L Q E H Y G S
L K G L T L S W I G D G N N I L H S I M M S A A K F G M H L Q A A T P K G Y E P D
P N I V K L A E Q Y A K E N G T R L S M T N D P L E A A R G G N V L I T D T W I S
M G Q E D E K K K R L Q A F Q G Y Q V T M K T A K V A A S D W T F L H C L P R K
P E E V D D E V F Y S P R S L V F P E A E N R K W T I M A V M V S L L T D Y S P V
L Q K P K F
SEQ ID NO.: 3
Human Otc-Δ cDNA, deleted nucleotides are underlined.
gagccccagg actgagatat ttttactata ccttctctat catcttgcac ccccaaaata gcttccaggg cacttctatt
tgtttttgtg gaaagactgg caattagagg tagaaaagtg aaataaatgg aaatagtact actcagggct gtcacatcta
catctgtgtt tttgcagtgc
caatttgcat tttctgagtg agttacttct actcaccttc acagcagcca gtaccgcagt gccttgcata tattatatcc
tcaatgagta cttgtcaatt gattttgtac atgcgtgtga cagtataaat atattatgaa aaatgaggag gccaggcaat
aaaagagtca ggatttcttc
caaaaaaaat acacagcggt ggagcttggc ataaagttca aatgctccta caccctgccc tgcagtatct
ctaaccaggg gactttgata aggaagctga agggtgatat tacctttgct ccctcactgc aactgaacac atttcttagt
ttttaggtgg cccccgctgg ctaacttgct
gtggagtttt caagggcata gaatcgtcct ttacacaatt aaaagaagat gctgtttaat ctgaggatcc tgttaaacaa
tgcagctttt agaaatggtc acaacttcat ggttcgaaat tttcggtgtg gacaaccact acaaaataaa gtgcagctga
agggccgtga ccttctcact
ctaaaaaact ttaccggaga agaaattaaa tatatgctat ggctatcagc agatctgaaa tttaggataa aacagaaagg
agagtatttg cctttattgc aagggaagtc cttaggcatg atttttgaga aaagaagtac tcgaacaaga ttgtctacag
aaacaggctt tgcacttctg
ggaggacatc cttgttttct taccacacaa gatattcatt tgggtgtgaa tgaaagtctc acggacacgg cccgtgtatt
gtctagcatg gcagatgcag tattggctcg agtgtataaa caatcagatt tggacaccct tgctaaagaa gcatccatcc
caattatcaa tgggctgtca
gatttgtacc atcctatcca gatcctggct gattacctca cgctccagga acactatagc tctctgaaag gtcttaccct
cagctggatc ggggatggga acaatatcct gcactccatc atgatgagcg cagcgaaatt cggaatgcac
cttcaggcag ctactccaaa gggttatgag ccggatgcta gtgtaaccaa gttggcagag cagtatgcca
aagagaatgg taccaagctg ttgctgacaa atgatccatt ggaagcagcg catggaggca atgtattaat tacagacact
tggataagca tgggacaaga agaggagaag aaaaagcggc tccaggcttt ccaaggttac caggttacaa
tgaagactgc taaagttgct gcctctgact ggacattttt acactgcttg cccagaaagc cagaagaagt ggatgatgaa
gtcttttatt ctcctcgatc actagtgttc ccagaggcag aaaacagaaa gtggacaatc atggctgtca tggtgtccct
gctgacagat tactcacctc agctccagaa gcctaaattt tgatgttgtg ttacttgtca agaaagaagc aatgttcttc
agtaacagaa tgagttggtt tatggggaaa agagaagaga atctaaaaaa taaacaaatc cctaacacgt
ggtatgggtg aaccgtatga tatgctttgc cattgtgaaa ctttccttaa gcctttaatt taagtgctga tgcactgtaa
tacgtgctta actttgctta aactctctaa ttcccaattt ctgagttaca tttagatatc atattaatta tcatatacat ttacttc
SEQ ID NO.: 4
Human Otc-Δ protein, deleted amino acids are underlined.
MLFNLRILLN NAAFRNGHNF MVRNFRCGQP LQNKVQLKGR DLLTLKNFTG EEIKYMLWLS
ADLKFRIKQK GEYLPLLQGK SLGMIFEKRS TRTRLSTETG FALLGGHPCF PTTQDIHLGV
NESLTDTARV LSSMADAVLA RVYKQSDLDT LAKEASIPII NGLSDLYHPI QILADYLTLQ
EHYSSLKGLT LSCFGDGNNI LHSIMMSAAK FGMHLQAATP KGYEPDASVT KLAEQYAKEN
GTKLLLTNDP LEAAHGGNVL ITDTWISMGR EEEKKKRLQA FQGYQVTMKT AKVAASDWTF
LHCLPRKPEE VDDEVFYSPR SLVFPEAENR KWTIMAVMVS LLTDYSPQLQ KPKF
SEQ ID NO.: 5
Porcine Otc-Δ cDNA, deleted nucleotides are underlined.
Ggtggacaaccactacaaaataaagtgcagctgaagggtcgtgacctcctcactctaaagaactttacaggagaaga
aattaagtatatcctatggctatcagctgatctgaaatttaggataaagcagaaaggagagtatttgcctttattgcaaggga
agtccctcggcatgatttttgaaaaaagaagtactcgaacaagattgtctacagaaacaggctttgcccttctaggaggac
acccttgttttcttaccacacaagatattcacttgggtgtgaatgaaagtctcaaggacaccgctcgtgtgttgtctagcatga
cagatgcagtgttggcgcgagtgtataaacaatcagacctggacatcctggctcaagaagcatccatcccaatcatcaat
gggctgtcagatttgtaccatcctatccagatcctggctgattacctcacgctccaggaacactacggcgctctgaaaggc
cttaccctcagctggattggggatgggaataacatcctgcactccatcatgatgagtgcggcgaaatttgggatgcaccttc
aggtggcgactccaaagggctatgagccggatcccagtataaccaagttggcggagcagtatgccaaggagaacggt
accaacgtgtcgctgacgaatgatccattggaggcggctcgtggaggcaatgtattaattacagacacttggataagcat
gggacaagaagaggagaagaaaaagcggctccaggctttccaaggttaccaggttacaatgaagactgcggaagtt
gctgcctctgactggacatttttacactgcctgcccagaaagccagaagaagtggacgatgaagtgttttactctccacaat
cacttgtattcccggaggctgaaaacagaaagtggacaatcatggctgtcatggtgtctctgctgacagattactcgcctca
gctccagaagccgaagttttgatgccgtgatgcttgtcaagagggaaaccatgttctcccataacagaatgagtcagttga
taagggaaggagaagagaatctaagaaataaacagatcc
SEQ ID NO.: 6
Porcine Otc-Δ protein, deleted amino acids are underlined.
GGQPLQNKVQ LKGRDLLTLK NFTGEEIKYI LWLSADLKFR IKQKGEYLPL
LQGKSLGMIF EKRSTRTRLS TETGFALLGG HPCFLTTQDI HLGVNESLKD
TARVLSSMTD AVLARVYKQS DLDILAQEAS IPIINGLSDL YHPIQILADY
LTLQEHYGAL KGLTLSWIGD GNNILHSIMM SAAKFGMHLQ VATPKGYEPD
PSITKLAEQY AKENGTNVSL TNDPLEAARG GNVLITDTWI SMGQEEEKKK
RLQAFQGYQV TMKTAEVAAS DWTFLHCLPR KPEEVDDEVF YSPQSLVFPE
AENRKWTIMA VMVSLLTDYS PQLQKPKF

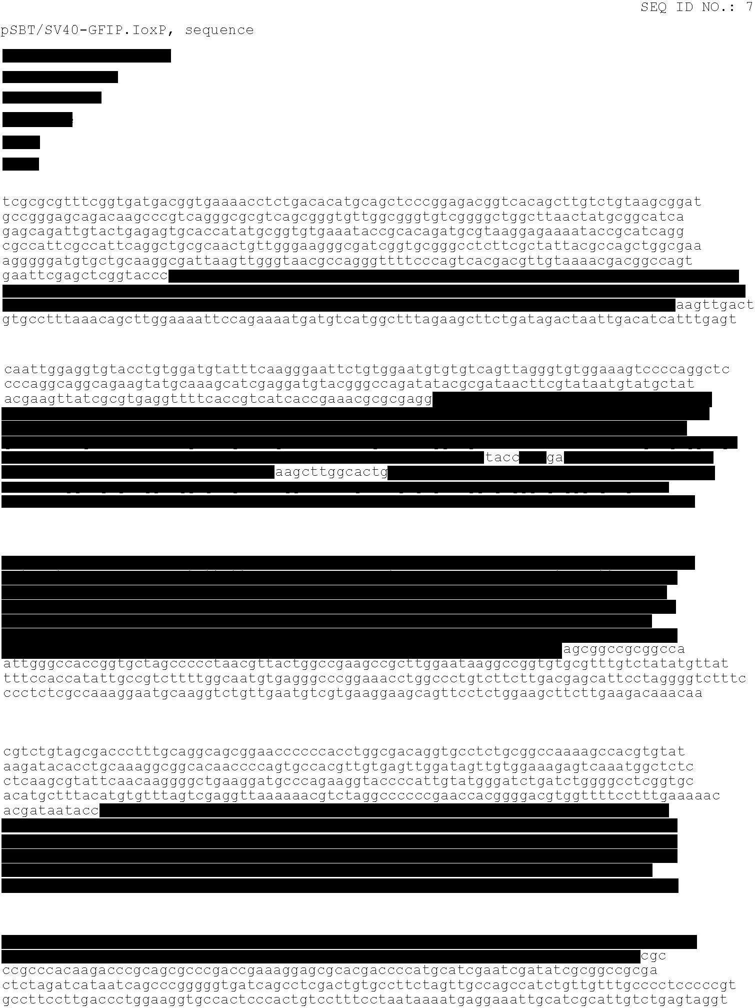

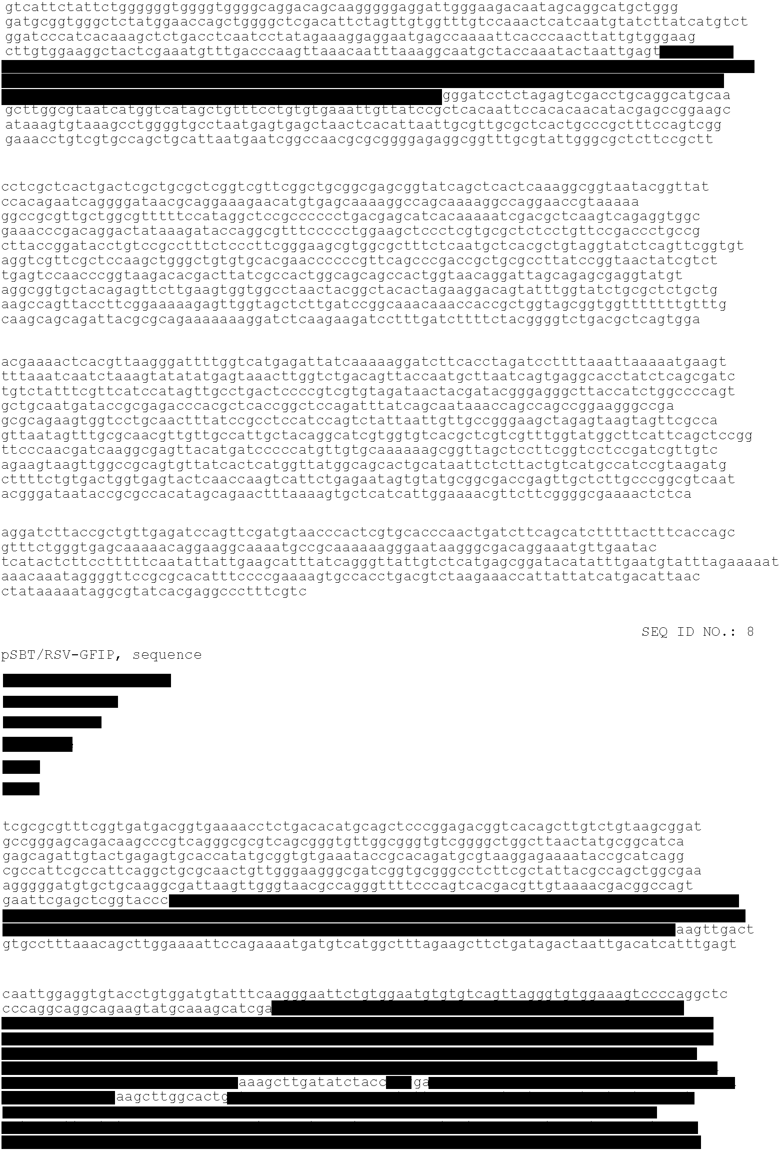

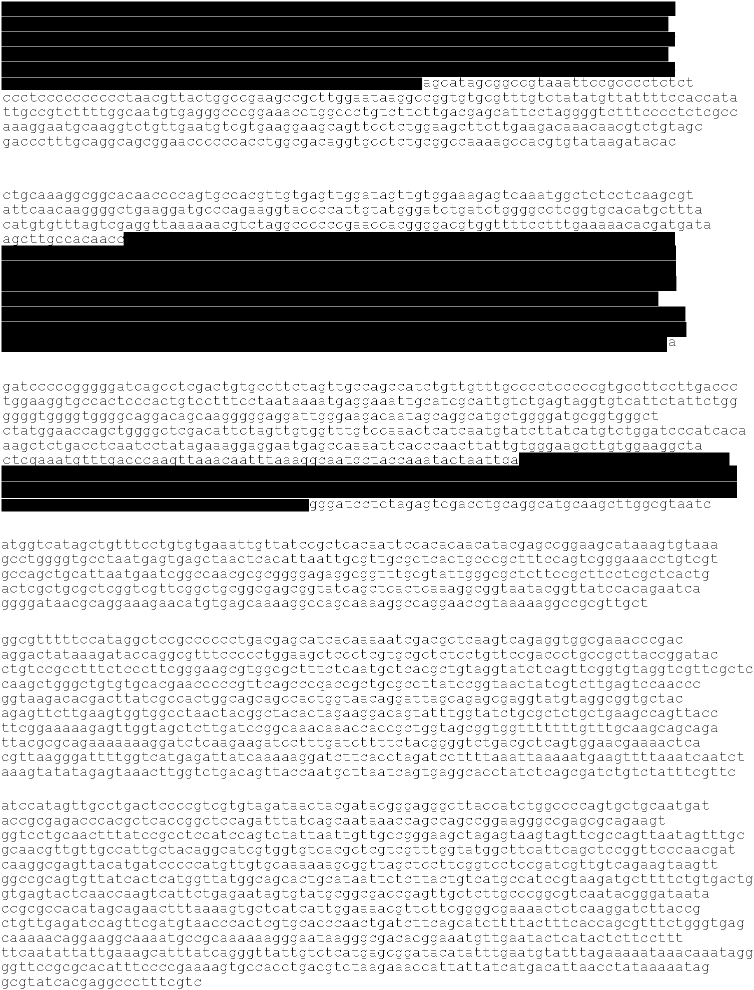

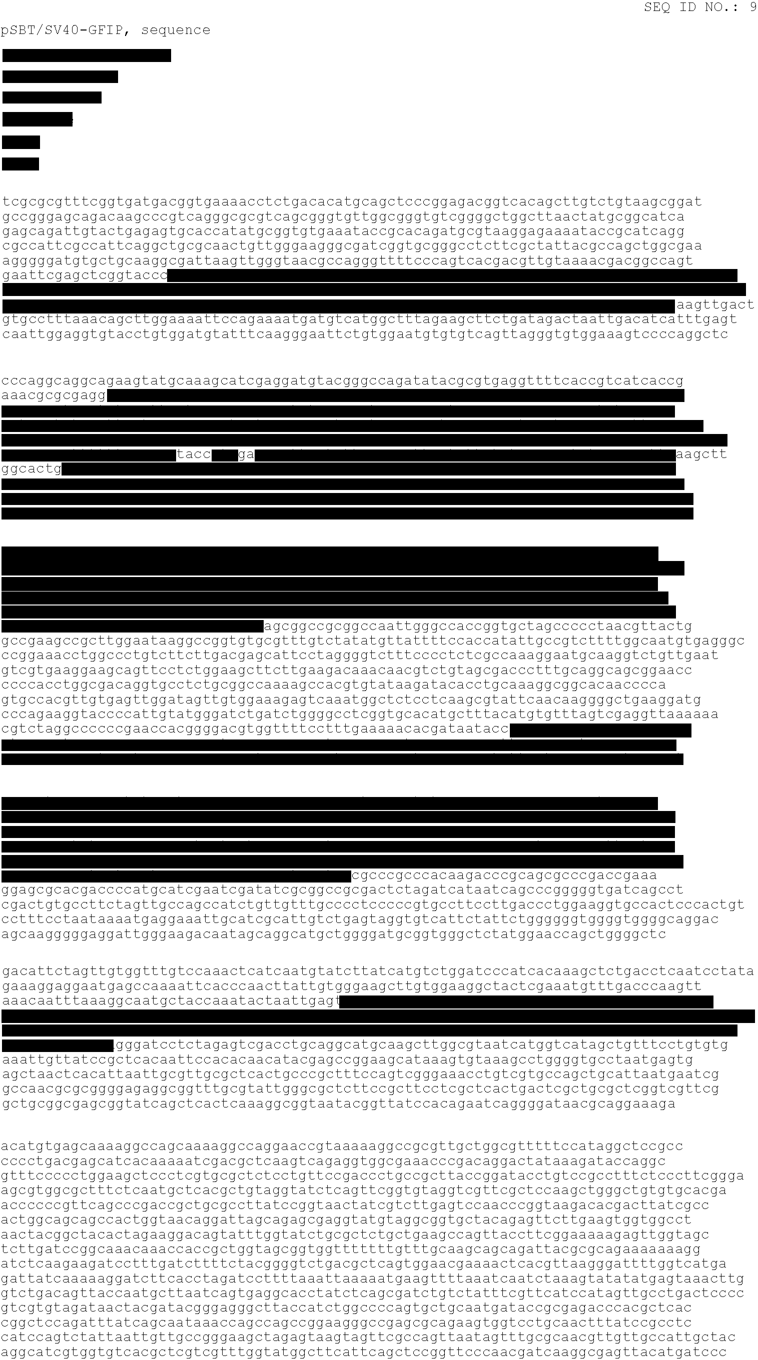

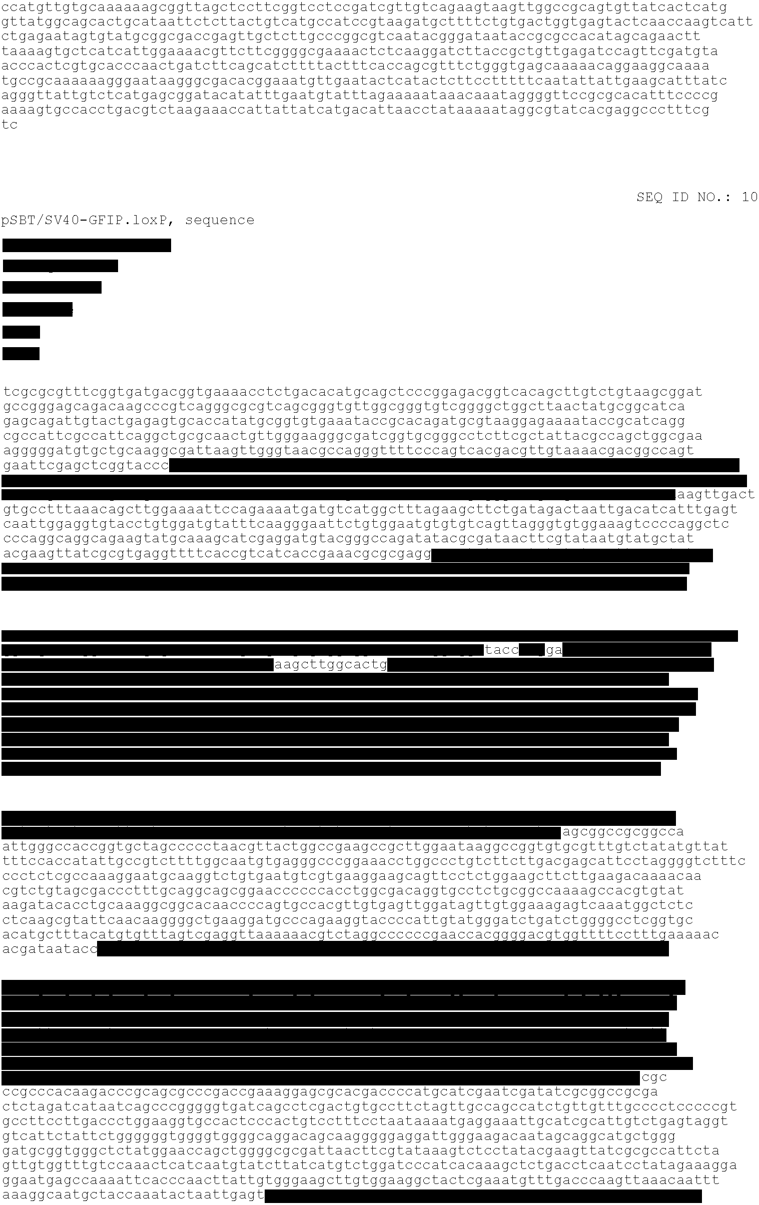

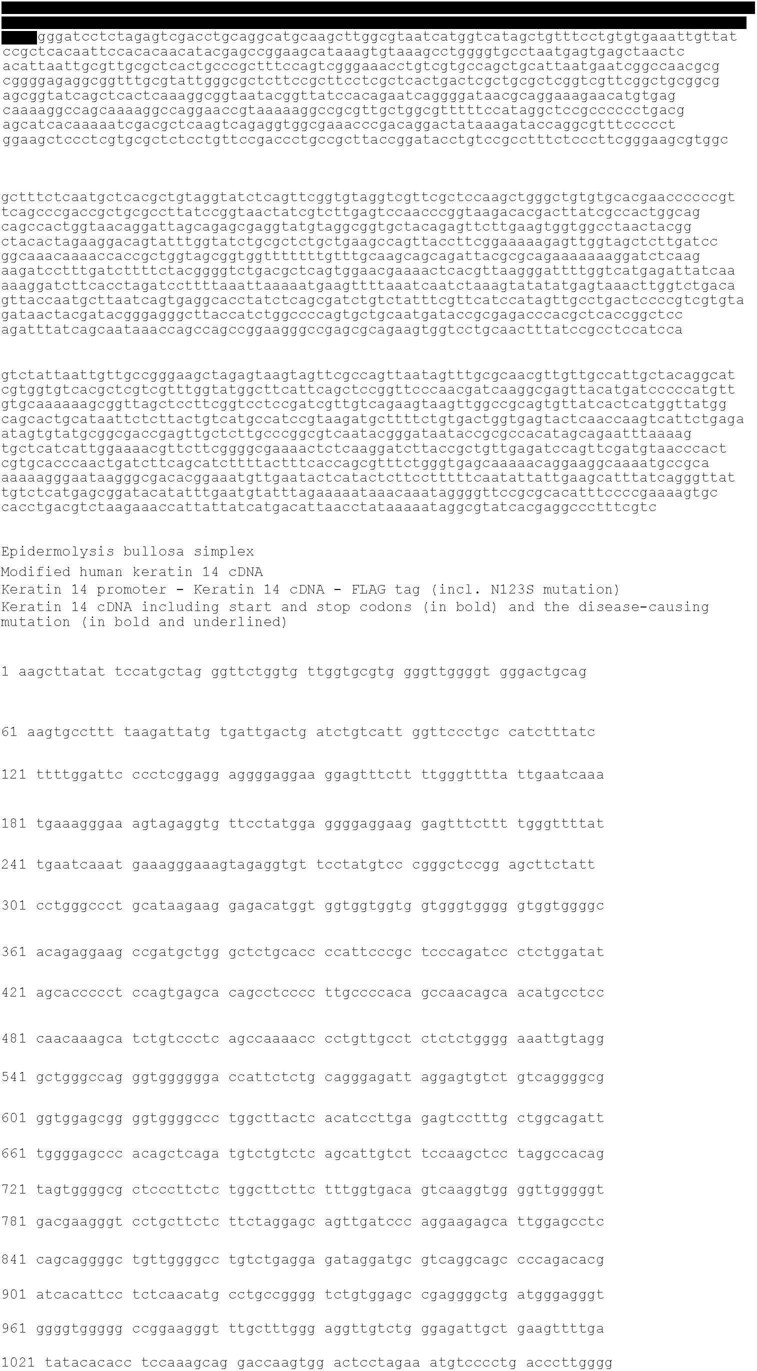

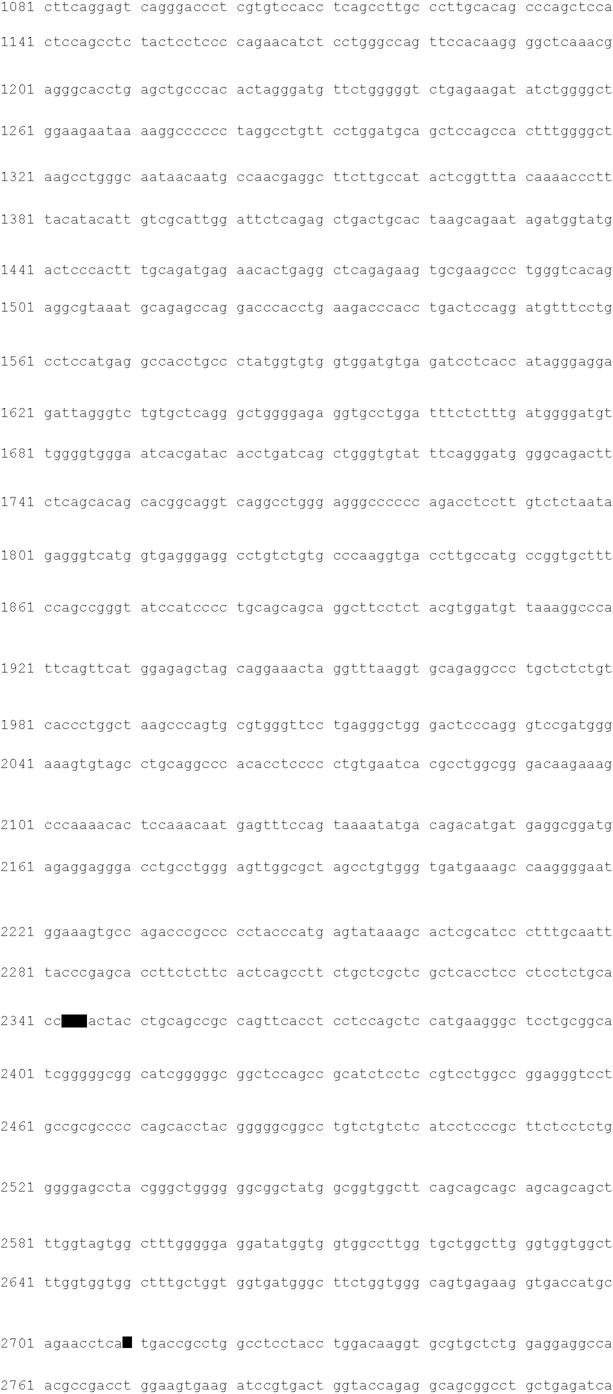

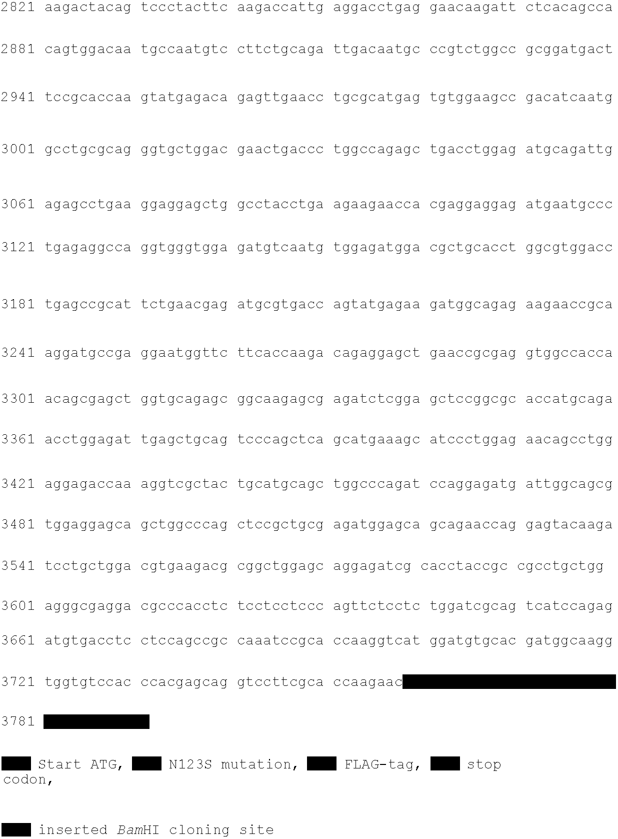

Modified human keratin 14 cDNA
keratin 14 cDNA including start and stop codons (in bold) and the
disease-causing mutation (in bold and underlined)
atgactac ctgcagccgc cagttcacct cctccagctc catgaagggc tctgcggcat
cgggggcggc atcgggggcg gctccagccg catctcctcc gtcctggccg gagggtcctg
ccgcgccccc agcacctacg ggggcggcct gtctgtctca tcctcccgct tctcctctgg
gggagcctac gggctggggg gcggctatgg cggtggcttc agcagcagca gcagcagctt
tggtagtggc tttgggggag gatatggtgg tggccttggt gctggcttgg gtggtggctt
tggtggtggc tttgctggtg gtgatgggct tctggtgggc agtgagaagg tgaccatgca
gaacctcaGt gaccgcctgg cctcctacct ggacaaggtg cgtgctctgg aggaggccaa
cgccgacctg gaagtgaaga tccgtgactg gtaccagagg cagcggcctg ctgagatcaa
agactacagt ccctacttca agaccattga ggacctgagg aacaagattc tcacagccac
agtggacaat gccaatgtcc ttctgcagat tgacaatgcc cgtctggccg cggatgactt
ccgcaccaag tatgagacag agttgaacct gcgcatgagt gtggaagccg acatcaatgg
cctgcgcagg gtgctggacg aactgaccct ggccagagct gacctggaga tgcagattga
gagcctgaag gaggagctgg cctacctgaa gaagaaccac gaggaggaga tgaatgccct
gagaggccag gtgggtggag atgtcaatgt ggagatggac gctgcacctg gcgtggacct
gagccgcatt ctgaacgaga tgcgtgacca gtatgagaag atggcagaga agaaccgcaa
ggatgccgag gaatggttct tcaccaagac agaggagctg aaccgcgagg tggccaccaa
cagcgagctg gtgcagagcg gcaagagcga gatctcggag ctccggcgca ccatgcagaa
cctggagatt gagctgcagt cccagctcag catgaaagca tccctggaga acagcctgga
ggagaccaaa ggtcgctact gcatgcagct ggcccagatc caggagatga ttggcagcgt
ggaggagcag ctggcccagc tccgctgcga gatggagcag cagaaccagg agtacaagat
cctgctggac gtgaagacgc ggctggagca ggagatcgcc acctaccgcc gcctgctgga
gggcgaggac gcccacctct cctcctccca gttctcctct ggatcgcagt catccagaga
tgtgacctcc tccagccgcc aaatccgcac caaggtcatg gatgtgcacg atggcaaggt
ggtgtccacc cacgagcagg tccttcgcac caagaactga ggctgcccag ccccgctcag
gcctaggagg ccccccgtgt ggacac
Keratin 14 human protein
MTTCSRQFTS SSSMKGSCGI GGGIGGGSSR ISSVLAGGSC RAPSTYGGGL
SVSSSRFSSG
GAYGLGGGYG GGFSSSSSSF GSGFGGGYGG GLGAGLGGGF GGGFAGGDGL
LVGSEKVTMQ
NLNDRLASYL DKVRALEEAN ADLEVKIRDW YQRQRPAEIK DYSPYFKTIE
DLRNKILTAT
VDNANVLLQI DNARLAADDF RTKYETELNL RMSVEADING LRRVLDELTL
ARADLEMQIE
SLKEELAYLK KNHEEEMNAL RGQVGGDVNV EMDAAPGVDL SRILNEMRDQ
YEKMAEKNRK
DAEEWFFTKT EELNREVATN SELVQSGKSE ISELRRTMQN LEIELQSQLS
MKASLENSLE
ETKGRYCMQL AQIQEMIGSV EEQLAQLRCE MEQQNQEYKI LLDVKTRLEQ
EIATYRRLLE
GEDAHLSSSQ FSSGSQSSRD VTSSSRQIRT KVMDVHDGKV VSTHEQVLRT KN
Modified Keratin 14 human protein, with the disease-causing
mutation underlined in bold
MTTCSRQFTS SSSMKGSCGI GGGIGGGSSR ISSVLAGGSC RAPSTYGGGL
SVSSSRFSSG GAYGLGGGYG GGFSSSSSSF GSGFGGGYGG GLGAGLGGGF
GGGFAGGDGL LVGSEKVTMQ NLSDRLASYL DKVRALEEAN ADLEVKIRDW
YQRQRPAEIK DYSPYFKTIE DLRNKILTAT VDNANVLLQI DNARLAADDF
RTKYETELNL RMSVEADING LRRVLDELTL ARADLEMQIE SLKEELAYLK
KNHEEEMNAL RGQVGGDVNV EMDAAPGVDL SRILNEMRDQ YEKMAEKNRK
DAEEWFFTKT EELNREVATN SELVQSGKSE ISELRRTMQN LEIELQSQLS
MKASLENSLE ETKGRYCMQL AQIQEMIGSV EEQLAQLRCE MEQQNQEYKI
LLDVKTRLEQ EIATYRRLLE GEDAHLSSSQ FSSGSQSSRD VTSSSRQIRT
KVMDVHDGKV VSTHEQVLRT KN

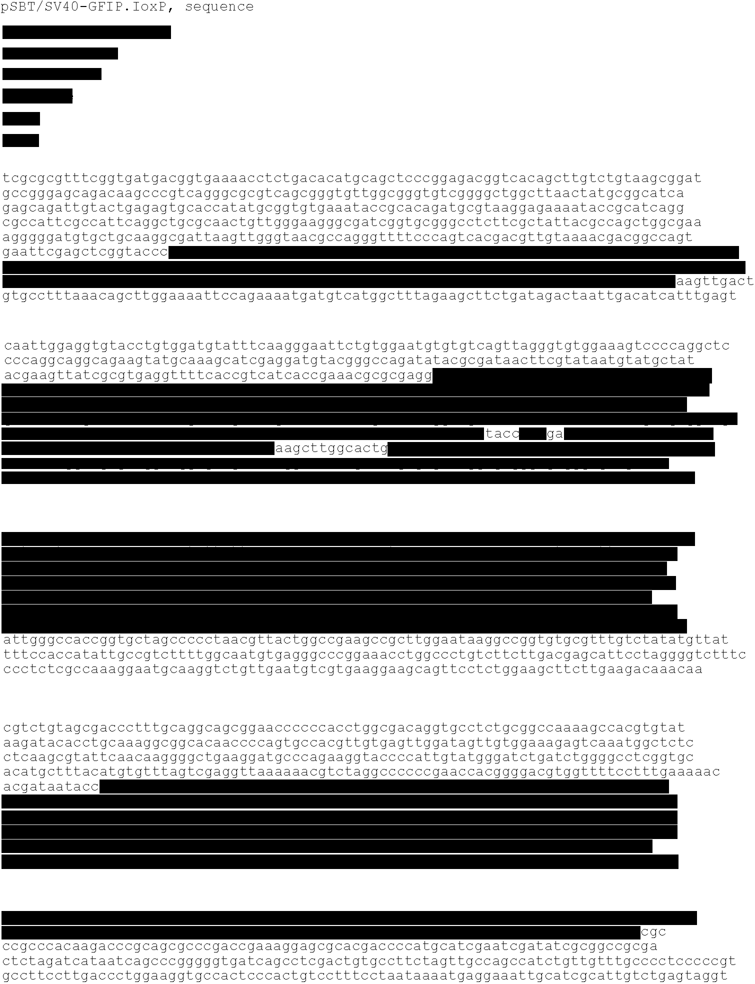

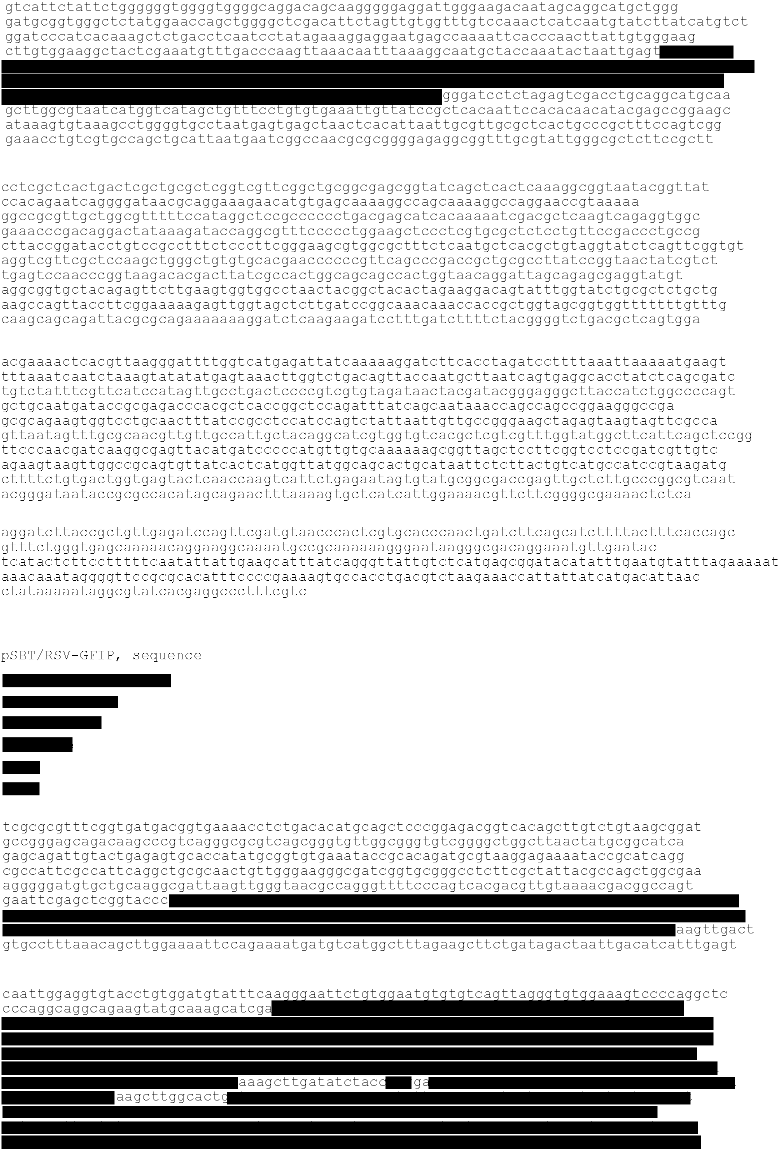

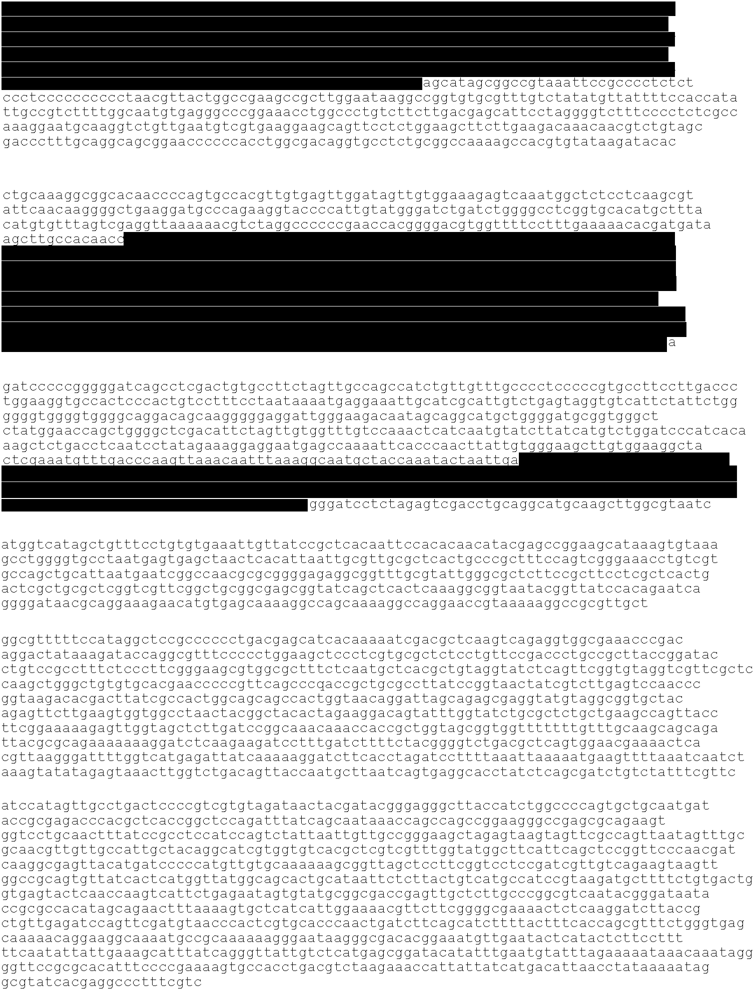

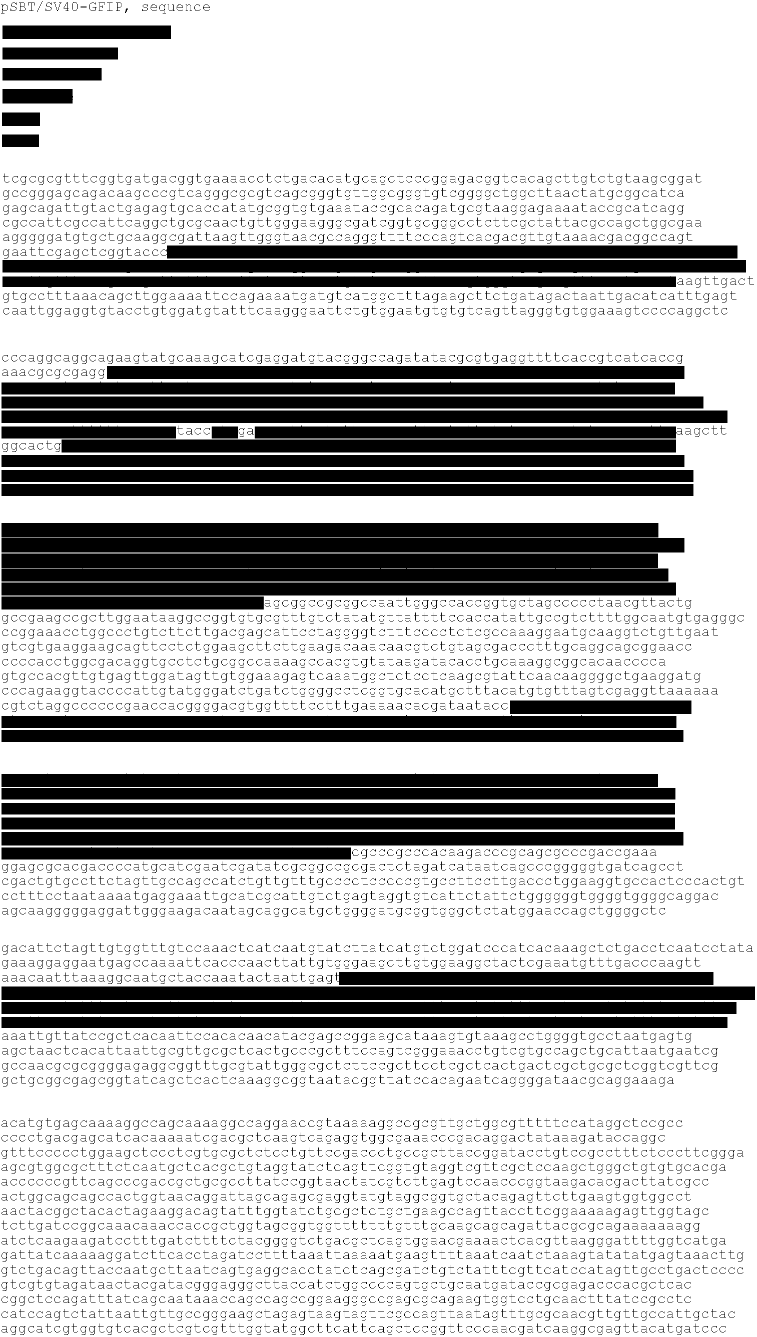

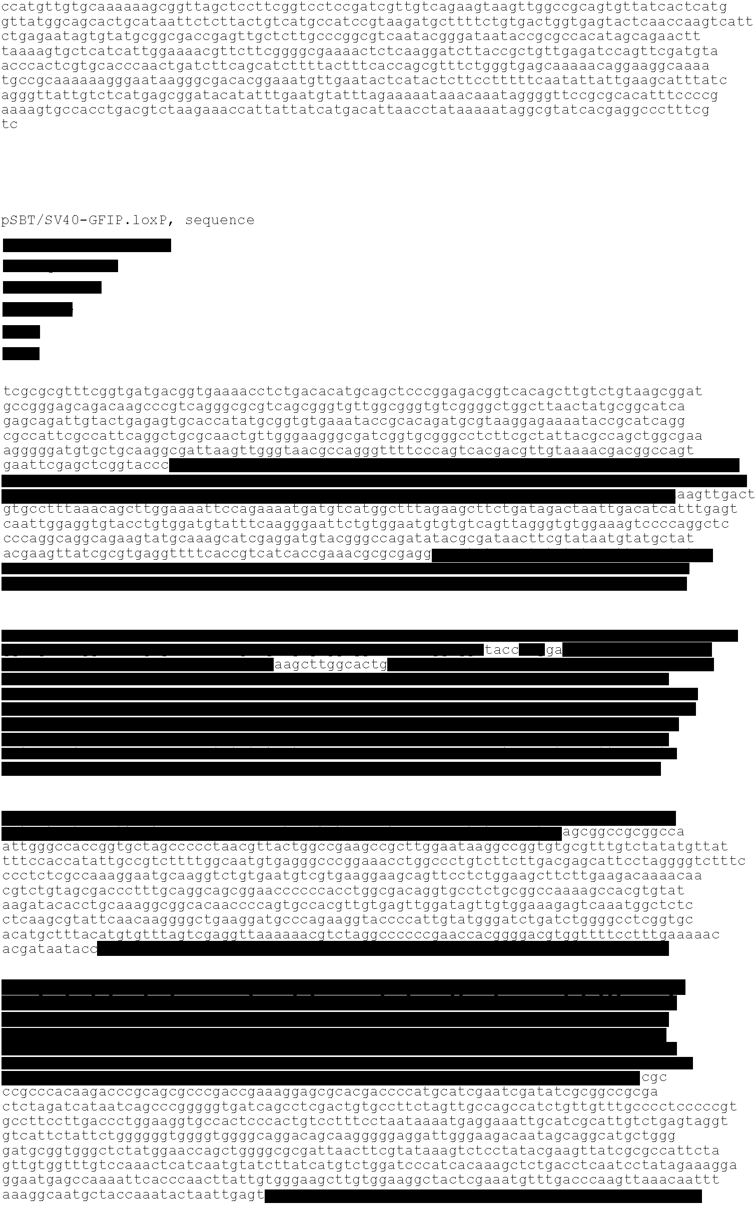

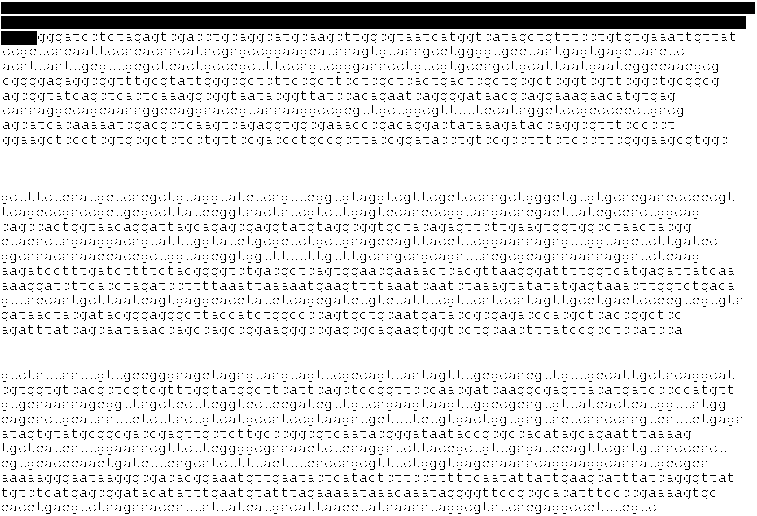

1. A genetically modified pig as a model for studying breast cancer, wherein the modified pig expresses at least one phenotype associated with said breast cancer; and/or
a modified pig comprising at least one modified endogenous nucleic acid sequence selected from:
i) exon 3 or part thereof of a BRCA1 gene,
ii) porcine BRCA1 gene or part thereof comprising a nucleotide substitution from T to G resulting in amino acid substitution from Cys to Gly at codon 61 of exon 3,
iii) exon 11 or part thereof of the BRCA1 gene,
iv) porcine BRCA1 gene or part thereof comprising a deletion of at least one allele of exon 11 or part thereof of the BRCA1 gene,
v) exon 11 or part thereof of the BRCA2 gene, and
vi) porcine BRCA2 gene comprising a deletion of at least one allele of exon 11 or part thereof of the BRCA2 gene, or
a transcriptional and/or translational product or part thereof.

2. The genetically modified pig according to claim 1, wherein the pig is a mini-pig.

3.-7. (canceled)

8. The genetically modified pig according to claim 1, wherein said pig is transgenic due to at least one mutation in exon 3 or part thereof of the BRCA1 gene, transcriptional and/or translational product or part thereof.

9. The genetically modified pig according to claim 8, wherein said mutation is a nucleotide substitution from T to G resulting in amino acid substitution from Cys to Gly at codon 61 of exon 3, transcriptional and/or translational product or part thereof.

10. (canceled)

11. (canceled)

12. The genetically modified pig according to claim 1, wherein said pig is transgenic due to at least one mutation in exon 11 or part thereof of the BRCA2 gene, transcriptional and/or translational product or part thereof.

13. The genetically modified pig according to claim 12, wherein said mutation is a deletion of at least one allele of exon 11 or part thereof of the BRCA 2 gene, transcriptional and/or translational product or part thereof.

14. (canceled)

15. The genetically modified pig according to claim 12, wherein said mutation is introduced into the endogenous porcine BRCA 2 gene by homologous recombination.

16. The genetically modified pig according to claim 1, wherein said pig is transgenic due to at least one mutation in exon 11 or part thereof of the BRCA 1 gene, transcriptional and/or translational product or part thereof.

17.-19. (canceled)

20. The genetically modified pig according to claim 1, wherein said pig is transgenic due to at least one mutation in exon 3 or part thereof of the BRCA1 gene, at least one mutation in exon 11 or part thereof of the BRCA 1 gene and at least one mutation in exon 11 or part thereof of the BRCA 2 gene, transcriptional and/or translational product or part thereof.

21. The genetically modified pig according to claim 1, wherein said at least one phenotype is selected from the group consisting of unilateral breast cancer, bilateral breast cancer, secondary tumours for example in the lymph nodes in the axilla, and secondary tumours for example in liver or lung.

22. (canceled)

23. (canceled)

24. A method for producing a transgenic pig, porcine blastocyst, embryo, fetus and/or donor cell as a model for breast cancer comprising:
i) establishing at least one oocyte
ii) separating the oocyte into at least three parts whereby at least one cytoplast is obtained,
iii) establishing a donor cell or membrane surrounded cell nucleus having genetic properties that produce a phenotypic or genetic modification according to claim 1,
iv) fusing at least one cytoplast with the donor cell or membrane surrounded cell nucleus,
v) obtaining a reconstructed embryo,
vi) activating the reconstructed embryo to form an embryo and culturing said embryo, and
vii) transferring said cultured embryo to a host mammal such that the embryo develops into a genetically modified fetus,
wherein said transgenic embryo is produced by a method comprising steps i) to v) and optionally vi),
wherein said transgenic blastocyst is produced by a method comprising steps i) to vi) and optionally vii), and
wherein said transgenic fetus is produced by a method comprising steps i) to vii).

25. A genetically modified porcine blastocyst derived from the genetically modified pig as defined in claim 1
and/or
a modified porcine blastocyst comprising at least one modified endogenous nucleic acid sequence selected from:
i) exon 3 or part thereof of a BRCA1 gene,
ii) porcine BRCA1 gene or part thereof comprising a nucleotide substitution from T to G resulting in amino acid substitution from Cys to Gly at codon 61 of exon 3,
iii) exon 11 or part thereof of the BRCA1 gene,
iv) porcine BRCA1 gene or part thereof comprising a deletion of at least one allele of exon 11 or part thereof of the BRCA1 gene,
v) exon 11 or part thereof of the BRCA2 gene, and
vi) porcine BRCA2 gene comprising a deletion of at least one allele of exon 11 or part thereof of the BRCA2 gene, or
a transcriptional and/or translational product or part thereof.

26. A genetically modified porcine embryo derived from the genetically modified pig as defined in claim 1
and/or
a modified porcine embryo comprising at least one modified endogenous nucleic acid sequence selected from:
i) exon 3 or part thereof of a BRCA1 gene,
ii) porcine BRCA1 gene or part thereof comprising a nucleotide substitution from T to G resulting in amino acid substitution from Cys to Gly at codon 61 of exon 3,
iii) exon 11 or part thereof of the BRCA1 gene,
iv) porcine BRCA1 gene or part thereof comprising a deletion of at least one allele of exon 11 or part thereof of the BRCA1 gene,
v) exon 11 or part thereof of the BRCA2 gene, and
vi) porcine BRCA2 gene comprising a deletion of at least one allele of exon 11 or part thereof of the BRCA2 gene, or
a transcriptional and/or translational product or part thereof.

27. A genetically modified porcine fetus derived from the genetically modified pig as defined in claim 1
and/or
a modified porcine fetus comprising at least one modified endogenous nucleic acid sequence selected from:
i) exon 3 or part thereof of a BRCA1 gene,
ii) porcine BRCA1 gene or part thereof comprising a nucleotide substitution from T to G resulting in amino acid substitution from Cys to Gly at codon 61 of exon 3,
iii) exon 11 or part thereof of the BRCA1 gene,
iv) porcine BRCA1 gene or part thereof comprising a deletion of at least one allele of exon 11 or part thereof of the BRCA1 gene,
v) exon 11 or part thereof of the BRCA2 gene, and
vi) porcine BRCA2 gene comprising a deletion of at least one allele of exon 11 or part thereof of the BRCA2 gene, or
a transcriptional and/or translational product or part thereof.

28. A genetically modified porcine donor cell and/or cell nucleus derived from the genetically modified pig as defined in claim 1
and/or
a modified porcine donor cell and/or cell nucleus comprising at least one modified endogenous nucleic acid sequence selected from:
i) exon 3 or part thereof of a BRCA1 gene,
ii) porcine BRCA1 gene or part thereof comprising a nucleotide substitution from T to G resulting in amino acid substitution from Cys to Gly at codon 61 of exon 3,
iii) exon 11 or part thereof of the BRCA1 gene,
iv) porcine BRCA1 gene or part thereof comprising a deletion of at least one allele of exon 11 or part thereof of the BRCA1 gene,
v) exon 11 or part thereof of the BRCA2 gene, and
vi) porcine BRCA2 gene comprising a deletion of at least one allele of exon 11 or part thereof of the BRCA2 gene, or
a transcriptional and/or translational product or part thereof.

29. The genetically modified pig model, porcine blastocyst, embryo, fetus and/or donor cell according to claim 24 obtainable by nuclear transfer comprising the steps of
i) establishing at least one oocyte having at least a part of a modified zona pellucida,
ii) separating the oocyte into at least two parts whereby an oocyte having a membrane surrounded nucleus and at least one cytoplast is obtained,
iii) establishing a donor cell or cell nucleus with genetic properties of the blastocyst, embryo, fetus, or donor cell of claim 24,
iv) fusing said at least one cytoplast with the donor cell or membrane surrounded cell nucleus,
v) obtaining a reconstructed embryo,
vi) activating the reconstructed embryo to form an embryo and culturing said embryo, and
vii) transferring said cultured embryo to a host mammal such that the embryo develops into a genetically modified fetus,
wherein said genetically modified embryo is obtainable by nuclear transfer comprising steps i) to v) and optionally vi), wherein said genetically modified blastocyst is obtainable by nuclear transfer comprising steps i) to vi) and optionally vii), and
wherein said genetically modified fetus is obtainable by nuclear transfer comprising steps i) to vii).

30. A method for producing a transgenic pig as a model for breast cancer comprising:
i) establishing at least one oocyte
ii) separating the oocyte into at least three parts whereby at least one cytoplast is obtained,
iii) establishing a donor cell or membrane surrounded cell nucleus having genetic properties that produce a phenotypic or genetic modification according to claim 1,
iv) fusing said at least one cytoplast with the donor cell or membrane surrounded cell nucleus,
v) obtaining a reconstructed embryo,
vi) activating the reconstructed embryo to form an embryo and culturing said embryo, and
vii) transferring said cultured embryo to a host mammal such that the embryo develops into a genetically modified fetus.

31.-42. (canceled)

43. A method for evaluating the effect of a therapeutic treatment of breast cancer, said method comprising the steps of
i) providing the modified pig according to claims 1,
ii) treating said pig with a pharmaceutical composition exerting an effect on said phenotype, and
iii) evaluating the effect observed.

44. (canceled)

45. A method for screening the efficacy of a pharmaceutical composition, said method comprising the steps of
i) providing the modified pig according to claims 1,
ii) expressing in said pig said genetic determinant and exerting said phenotype,
iii) administering to said pig a pharmaceutical composition the efficacy of which is to be evaluated, and
iv) evaluating the effect, if any, of the pharmaceutical composition on the phenotype exerted by the genetic determinant when expressed in the pig.

46. A method for treatment of a human being suffering from breast cancer, said method comprising the initial steps of
i) providing the modified pig according to claims 1,
ii) expressing in said pig said genetic determinant and exerting said phenotype,
iii) administering to said pig a pharmaceutical composition the efficacy of which is to be evaluated, and
iv) evaluating the effect observed, and
v) treating said human being suffering from breast cancer based on the effects observed in the pig model.

47.-129. (canceled)

130. A genetically modified pig as a model for studying breast cancer, the modified pig expressing at least one phenotype associated with breast cancer; wherein the modified pig comprises at least one modified endogenous nucleic acid sequence selected from:
i) exon 3 or part thereof of a BRCA1 gene,
ii) porcine BRCA1 gene or part thereof comprising a nucleotide substitution from T to G resulting in amino acid substitution from Cys to Gly at codon 61 of exon 3,
iii) exon 11 or part thereof of the BRCA1 gene,
iv) porcine BRCA1 gene or part thereof comprising a deletion of at least one allele of exon 11 or part thereof of the BRCA1 gene,
v) exon 11 or part thereof of the BRCA2 gene, and
vi) porcine BRCA2 gene comprising a deletion of at least one allele of exon 11 or part thereof of the BRCA2 gene, or
a transcriptional and/or translational product or part thereof.
